(19) United States
(12) Patent Application Publication
(10) Pub. No.: US 2026/0327589 A1
(43) Pub. Date: Oct. 22, 2026

(54) GLUCOAMYLASE AND METHODS OF USE THEREOF

(71) Applicant: DANISCO US INC., Palo Alto, CA (US)

(72) Inventors: Zhongmei TANG, Shanghai (CN); Zhenghong ZHANG, Shanghai (CN); Jing GE, Shanghai (CN); Lilia BABE, Emerald Hills, CA (US); Xingxiang XI, Shanghai (CN); Helong HAO, Shanghai (CN); Chao HUANG, Shanghai (CN)

(21) Appl. No.: 19/459,731

(22) Filed: Jan. 26, 2026

Related U.S. Application Data
(63) Continuation of application No. 17/719,518, filed on Apr. 13, 2022, now Pat. No. 12,559,738.
Application No. 17/719,518 is a continuation of application No. PCT/US2021/027894, filed on Apr. 19, 2021.

(51) Int. Cl. C12N 9/34; A23L 2/38; A23L 29/00; A23L 29/30; C12N 1/16; C12N 15/81; C12P 19/02; C12P 19/14

(52) U.S. Cl. CPC C12N 9/2428; A23L 2/382; A23L 29/06; A23L 29/35; C12N 1/16; C12N 15/81; C12P 19/02; C12P 19/14; C12Y 302/01003

(57) ABSTRACT

Described are a recombinant host cell, a composition comprising a glucoamylase and methods of saccharifying the starch substrate using the glucoamylase. Moreover, the disclosure also relates to a process of producing fermentation products and a method for increasing starch digestibility in an animal as well as a method of producing a fermented beverage.

### CROSS-REFERENCE TO RELATED APPLICATIONS

This application is a continuation of U.S. patent application Ser. No. 17/719,518, filed on Apr. 13, 2022, now U.S. Pat. No. 12,559,738, which is a continuation under 35 U.S.C. § 111(a) to International Patent Application No. PCT/US2021/027894, filed on Apr. 19, 2021, the disclosure of which is incorporated by reference herein in its entirety.

### INCORPORATION-BY-REFERENCE OF A MATERIAL SUBMITTED ON A READ-ONLY OPTICAL DISC, AS A TEXT FILE OR AN XML FILE VIA THE PATENT ELECTRONIC SYSTEM

The ST.26 sequence listing provided in the file named 20260626_NB41777-US-PCN3_ST26-ReplacementSL3.xml with a size of 255,179 bytes which was created on Jun. 26, 2026 and which is filed herewith, is incorporated by reference herein in its entirety.

### FIELD OF THE INVENTION

The present disclosure relates to a recombinant host cell, a composition comprising a glucoamylase and methods of saccharifying the starch substrate using the glucoamylase. Moreover, the disclosure also relates to a process of producing fermentation products and a method for increasing starch digestibility in an animal as well as a method of producing a fermented beverage.

### BACKGROUND

Glucoamylase (1,4-alpha-D-glucan glucohydrolase, EC 3.2.1.3) is an enzyme which catalyzes the release of D-glucose from the non-reducing ends of starch or related oligo- and poly-saccharide molecules. Glucoamylases are produced by several filamentous fungi and yeast.

The major application of glucoamylase is the saccharification of partially processed starch/dextrin to glucose, which is an essential substrate for numerous fermentation processes. The glucose may then be converted directly or indirectly into a fermentation product using a fermenting organism. Examples of commercial fermentation products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds.

The end product may also be syrup. For instance, the end product may be glucose, but may also be converted, e.g., by glucose isomerase to fructose or a mixture composed almost equally of glucose and fructose. This mixture, or a mixture further enriched with fructose, is the most commonly used high fructose corn syrup (HFCS) commercialized throughout the world.

Glucoamylase for commercial purposes has traditionally been produced employing filamentous fungi, although a diverse group of microorganisms is reported to produce glucoamylase since they secrete large quantities of the enzyme extracellularly. However, commercially used fungal glucoamylases have certain limitations such as slow catalytic activity or lack of stability that increase process costs.

There continues to be a need for new glucoamylases to improve the efficiency of saccharification and provide a high yield in fermentation products.

### SUMMARY

The present disclosure relates to a recombinant host cell, a composition comprising a glucoamylase and methods of saccharifying the starch substrate using the glucoamylase. Moreover, the disclosure also relates to a process of producing fermentation products and a method for increasing starch digestibility in an animal as well as a method of producing a fermented beverage.
1. In one aspect, a method for saccharifying a starch substrate, comprising contacting the starch substrate with a glucoamylase selected from the group consisting of:
   (a) a polypeptide having an amino acid sequence at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 61 or SEQ ID NO:142;
   (b) a polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 81; and
   (c) a polypeptide comprising one or more sequence motifs selected from the group consisting of:
      (i) YXₐX_{b}TXXX_{c}X_{d} (SEQ ID NO: 113), wherein X is any amino acid and Xₐ is N or S; X_{b} is T, S, or R; X_{c} is G or N; and X_{d} is D, N, or S;
      (ii) YNTTXAGD (SEQ ID NO: 114), wherein X is any amino acid;
      (iii) XₐX_{b}X_{c}X_{c}AANXX_{d} (SEQ ID NO: 115), wherein X is any amino acid and Xₐ is S or A; X_{b} is T, N, or V; X_{c} is L or I; and X_{d} is A or G;
      (iv) STLIAANXA (SEQ ID NO: 116), wherein wherein X is any amino acid;
      (v) XₐGXGNX_{b}X_{c} (SEQ ID NO: 117), wherein X is any amino acid and Xₐ is N or D; X_{b} is S or G; and X_{c} is Q, K, or E; and
      (vi) NGNGNSQ (SEQ ID NO: 118);
      wherein the polypeptide has at least 70% identity to the catalytic domain of SEQ ID NO: 61.
2. In some embodiments of the method of paragraph 1, wherein the polypeptide comprises a substitution, deletion or addition at a position corresponding to position 102 of the polypeptide of SEQ ID NO: 61.
3. In some embodiments of the method of paragraph 2, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.
4. In some embodiments of the method of any one of paragraphs 1-3, the polypeptide comprises a substitution, deletion or addition at a position corresponding to position 66 of the polypeptide of SEQ ID NO: 61.
5. In some embodiments of the method of paragraph 4, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H.
6. In some embodiments of the method of any one of paragraphs 1-5, wherein the polypeptide comprises SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:119, SEQ ID NO:120, SEQ ID NO:121, SEQ ID NO:122, SEQ ID NO:123, SEQ ID NO:126, SEQ ID NO:129, SEQ ID NO:132, SEQ ID NO:135, SEQ ID NO: 138, SEQ ID NO:140, SEQ ID NO:141, or SEQ ID NO:142.
7. In some embodiments of the method of any one of paragraphs 1-6, wherein the starch substrate is about 15% to 65%, 15% to 60% or 15% to 35% dry solid (DS).
8. In some embodiments of the method of any one of paragraphs 1-7, wherein the starch substrate comprises liquefied starch, gelatinized starch, or granular starch.
9. In some embodiments of the method of any one of paragraphs 1-8, further comprising adding a hexokinase, a xylanase, a glucose isomerase, a xylose isomerase, a phosphatase, a phytase, a pullulanase, a beta-amylase, an alpha-amylase, a protease, a cellulase, a hemicellulase, a lipase, a cutinase, a trehalase, an isoamylase, a redox enzyme, an esterase, a transferase, a pectinase, a hydrolase, an alpha-glucosidase, a beta-glucosidase, or a combination thereof to the starch substrate.
10. In some embodiments of the method of any one of paragraphs 1-9, wherein saccharifying the starch substrate results in a high glucose syrup comprising an amount of glucose selected from the group consisting of at least 95.5% glucose, at least 95.6% glucose, at least 95.7% glucose, at least 95.8% glucose, at least 95.9% glucose, at least 96% glucose, at least 96.1% glucose, at least 96.2% glucose, at least 96.3% glucose, at least 96.4% glucose, at least 96.5% glucose and at least 97% glucose.
11. In some embodiments of the method of any one of paragraphs 1-10, further comprising fermenting the high glucose syrup to an end product.
12. In some embodiments of the method of paragraph 11, wherein saccharifying and fermenting are carried out as a simultaneous saccharification and fermentation (SSF) process.
13. In some embodiments of the method of paragraph 11 or 12, wherein the end product is an alcohol, optionally, ethanol.
14. In some embodiments of the method of paragraph 11 or 12, wherein the end product is a biochemical selected from the group consisting of an amino acid, an organic acid, citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, lysine, itaconic acid, 1,3-propanediol, biodiesel, and isoprene.
15. In another aspect, a process of producing a fermentation product from a starch substrate comprising the steps of:
   1) liquefying the starch substrate;
   2) saccharifying the liquefied starch substrate; and
   3) fermenting with a fermenting organism;
   wherein step 2) is carried out using at least a glucoamylase selected from the group consisting of:
      a) a polypeptide having an amino acid sequence at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 61 or SEQ ID NO:142;
      b) a polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 81; and
      c) a polypeptide comprising one or more signature motifs selected from the group consisting of:
         (i) YXaXbTXXXcXd (SEQ ID NO: 113), wherein X is any amino acid and Xa is N or S; Xb is T, S, or R; Xc is G or N; and Xd is D, N, or S;
         (ii) YNTTXAGD (SEQ ID NO: 114), wherein X is any amino acid;
         (iii) XaXbXcXcAANXXd (SEQ ID NO: 115), wherein X is any amino acid and Xa is S or A; Xb is T, N, or V; Xc is L or I; and Xd is A or G;
         (iv) STLIAANXA (SEQ ID NO: 116), wherein wherein X is any amino acid;
         (v) XaGXGNXbXc (SEQ ID NO: 117), wherein X is any amino acid and Xa is N or D; Xb is S or G; and Xc is Q, K, or E; and
         (vi) NGNGNSQ (SEQ ID NO: 118);
         wherein the polypeptide has at least 70% identity to the catalytic domain of SEQ ID NO: 61.
16. In some embodiments of the process of paragraph 15, wherein the polypeptide comprises a substitution, deletion or addition at a position corresponding to position 102 of the polypeptide of SEQ ID NO: 61.
17. In some embodiments of the process of paragraph 16, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.
18. In some embodiments of the process of any one of paragraphs 15-17, the polypeptide comprises a substitution, deletion or addition at a position corresponding to position 66 of the polypeptide of SEQ ID NO: 61.
19. In some embodiments of the process of paragraph 18, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H.
20. In some embodiments of the process of any one of paragraphs 15-19, wherein the polypeptide comprises SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:119, SEQ ID NO:120, SEQ ID NO:121, SEQ ID NO:122, SEQ ID NO:123, SEQ ID NO:126, SEQ ID NO:129, SEQ ID NO:132, SEQ ID NO:135, SEQ ID NO: 138, SEQ ID NO:140, SEQ ID NO:141, or SEQ ID NO:142.
21. In another aspect, a process of producing a fermentation product from a starch substrate comprising the steps of:
   1) saccharifying the starch substrate at a temperature below the initial gelatinization temperature of the starch substrate; and
   2) fermenting with a fermenting organism, wherein step 1) is carried out using at least a glucoamylase selected from the group consisting of:
      a) a polypeptide having an amino acid sequence at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 61 or SEQ ID NO:142;
      b) a polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 81; and
      c) a polypeptide comprising one or more signature motifs selected from the group consisting of:
         (i) YXaXbTXXXcXd (SEQ ID NO: 113), wherein X is any amino acid and Xa is N or S; Xb is T, S, or R; Xc is G or N; and Xd is D, N, or S;
         (ii) YNTTXAGD (SEQ ID NO: 114), wherein X is any amino acid;
         (iii) XaXbXcXcAANXXd (SEQ ID NO: 115), wherein X is any amino acid and Xa is S or A; Xb is T, N, or V; Xc is L or I; and Xd is A or G;
         (iv) STLIAANXA (SEQ ID NO: 116), wherein wherein X is any amino acid;
         (v) XaGXGNXbXc (SEQ ID NO: 117), wherein X is any amino acid and Xa is N or D; Xb is S or G; and Xc is Q, K, or E; and
         (vi) NGNGNSQ (SEQ ID NO: 118);
         wherein the polypeptide has at least 70% identity to the catalytic domain of SEQ ID NO: 61.
22. In some embodiments of the process of paragraph 21, wherein the polypeptide comprises a substitution, deletion or addition at a position corresponding to position 102 of the polypeptide of SEQ ID NO: 61.
23. In some embodiments of the process of paragraph 22, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.
24. In some embodiments of the process of any one of paragraphs 21-23, the polypeptide comprises a substitution, deletion or addition at a position corresponding to position 66 of the polypeptide of SEQ ID NO: 61.
25. In some embodiments of the process of paragraph 24, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H. 26. In some embodiments of the process of any one of paragraphs 21-25, wherein the polypeptide comprises SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:119, SEQ ID NO:120, SEQ ID NO:121, SEQ ID NO:122, SEQ ID NO:123, SEQ ID NO:126, SEQ ID NO:129, SEQ ID NO:132, SEQ ID NO:135, SEQ ID NO: 138, SEQ ID NO:140, SEQ ID NO:141, or SEQ ID NO:142.
27. In another aspect, a method for increasing starch digestibility in an animal which comprises adding at least one glucoamylase selected from the group consisting of:
   a) a polypeptide having an amino acid sequence at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 61 or SEQ ID NO:142;
   b) a polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 81; and
   c) a polypeptide comprising one or more signature motifs selected from the group consisting of:
      (i) YXaXbTXXXcXd (SEQ ID NO: 113), wherein X is any amino acid and Xa is N or S; Xb is T, S, or R; Xc is G or N; and Xd is D, N, or S;
      (ii) YNTTXAGD (SEQ ID NO: 114), wherein X is any amino acid;
      (iii) XaXbXcXcAANXXd (SEQ ID NO: 115), wherein X is any amino acid and Xa is S or A; Xb is T, N, or V; Xc is L or I; and Xd is A or G;
      (iv) STLIAANXA (SEQ ID NO: 116), wherein wherein X is any amino acid;
      (v) XaGXGNXbXc (SEQ ID NO: 117), wherein X is any amino acid and Xa is N or D; Xb is S or G; and Xc is Q, K, or E; and
      (vi) NGNGNSQ (SEQ ID NO: 118);
      wherein the polypeptide has at least 70% identity to the catalytic domain of SEQ ID NO: 61.
28. In some embodiments of the method of paragraph 27, wherein the polypeptide comprises a substitution, deletion or addition at a position corresponding to position 102 of the polypeptide of SEQ ID NO: 61.
29. In some embodiments of the method of paragraph 28, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.
30. In some embodiments of the method of any one of paragraphs 27-29, the polypeptide comprises a substitution, deletion or addition at a position corresponding to position 66 of the polypeptide of SEQ ID NO: 61.
31. In some embodiments of the method of paragraph 30, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H.
32. In some embodiments of the method of any one of paragraphs 27-31, wherein the polypeptide comprises SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:119, SEQ ID NO:120, SEQ ID NO:121, SEQ ID NO:122, SEQ ID NO:123, SEQ ID NO:126, SEQ ID NO:129, SEQ ID NO:132, SEQ ID NO:135, SEQ ID NO: 138, SEQ ID NO:140, SEQ ID NO:141, or SEQ ID NO:142.
33. In another aspect, a method of producing a fermented beverage, wherein the method comprises the step of contacting a mash and/or a wort with a glucoamylase selected from the group consisting of:
   a) a polypeptide having an amino acid sequence at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 61 or SEQ ID NO:142;
   b) a polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 81; and
   c) a polypeptide comprising one or more signature motifs selected from the group consisting of:
      (i) YXaXbTXXXcXd (SEQ ID NO: 113), wherein X is any amino acid and Xa is N or S; Xb is T, S, or R; Xc is G or N; and Xd is D, N, or S;
      (ii) YNTTXAGD (SEQ ID NO: 114), wherein X is any amino acid;
      (iii) XaXbXcXcAANXXd (SEQ ID NO: 115), wherein X is any amino acid and Xa is S or A; Xb is T, N, or V; Xc is L or I; and Xd is A or G;
      (iv) STLIAANXA (SEQ ID NO: 116), wherein wherein X is any amino acid;
      (v) XaGXGNXbXc (SEQ ID NO: 117), wherein X is any amino acid and Xa is N or D; Xb is S or G; and Xc is Q, K, or E; and
      (vi) NGNGNSQ (SEQ ID NO: 118);
      wherein the polypeptide has at least 70% identity to the catalytic domain of SEQ ID NO: 61.
34. In some embodiments of the method of paragraph 33, wherein the polypeptide comprises a substitution, deletion or addition at a position corresponding to position 102 of the polypeptide of SEQ ID NO: 61.
35. In some embodiments of the method of paragraph 34, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.
36. In some embodiments of the method of any one of paragraphs 33-35, the polypeptide comprises a substitution, deletion or addition at a position corresponding to position 66 of the polypeptide of SEQ ID NO: 61.
37. In some embodiments of the method of paragraph 36, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H.
38. In some embodiments of the method of any one of paragraphs 33-37, wherein the polypeptide comprises SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:119, SEQ ID NO:120, SEQ ID NO:121, SEQ ID NO:122, SEQ ID NO:123, SEQ ID NO:126, SEQ ID NO:129, SEQ ID NO:132, SEQ ID NO:135, SEQ ID NO: 138, SEQ ID NO:140, SEQ ID NO:141, or SEQ ID NO:142.
39. In another aspect, a composition comprising a starch substrate and a glucoamylase selected from the group consisting of:
   a) a polypeptide having an amino acid sequence at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 61 or SEQ ID NO:142;
   b) a polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 81; and
   c) a polypeptide comprising one or more signature motifs selected from the group consisting of:
      (i) YXaXbTXXXcXd (SEQ ID NO: 113), wherein X is any amino acid and Xa is N or S; Xb is T, S, or R; X_{c} is G or N; and X_{d} is D, N, or S;
      (ii) YNTTXAGD (SEQ ID NO: 114), wherein X is any amino acid;
      (iii) XaXbXcXcAANXXd (SEQ ID NO: 115), wherein X is any amino acid and Xa is S or A; Xb is T, N, or V; Xc is L or I; and Xd is A or G;
      (iv) STLIAANXA (SEQ ID NO: 116), wherein wherein X is any amino acid;
      (v) XaGXGNXbXc (SEQ ID NO: 117), wherein X is any amino acid and Xa is N or D; Xb is S or G; and Xc is Q, K, or E; and
      (vi) NGNGNSQ (SEQ ID NO: 118);
      wherein the polypeptide has at least 70% identity to the catalytic domain of SEQ ID NO: 61; wherein said composition is at a temperature of about 4-40 °C and a pH of about 3-7.
40. In some embodiments of the composition of paragraph 39, wherein the polypeptide comprises a substitution, deletion or addition at a position corresponding to position 102 of the polypeptide of SEQ ID NO: 61.
41. In some embodiments of the composition of paragraph 40, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.
42. In some embodiments of the composition of any one of paragraphs 39-41, the polypeptide comprises a substitution, deletion or addition at a position corresponding to position 66 of the polypeptide of SEQ ID NO: 61.
43. In some embodiments of the composition of paragraph 42, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H.
44. In some embodiments of the composition of any one of paragraphs 39-43, wherein the polypeptide comprises SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:119, SEQ ID NO:120, SEQ ID NO:121, SEQ ID NO:122, SEQ ID NO:123, SEQ ID NO:126, SEQ ID NO:129, SEQ ID NO:132, SEQ ID NO:135, SEQ ID NO: 138, SEQ ID NO:140, SEQ ID NO:141, or SEQ ID NO:142.
45. In another aspect, a recombinant host cell comprising a glucoamylase selected from the group consisting of:
   a) a polypeptide having an amino acid sequence at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 61 or SEQ ID NO:142;
   b) a polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 81; and
   c) a polypeptide comprising one or more signature motifs selected from the group consisting of:
      (i) YXaXbTXXXcXd(SEQ ID NO: 113), wherein X is any amino acid and Xa is N or S; Xb is T, S, or R; Xc is G or N; and Xd is D, N, or S;
      (ii) YNTTXAGD (SEQ ID NO: 114), wherein X is any amino acid;
      (iii) XaXbXcXcAANXXd (SEQ ID NO: 115), wherein X is any amino acid and Xa is S or A; Xb is T, N, or V; Xc is L or I; and Xd is A or G;
      (iv) STLIAANXA (SEQ ID NO: 116), wherein wherein X is any amino acid;
      (v) XaGXGNXbXc (SEQ ID NO: 117), wherein X is any amino acid and Xa is N or D; Xb is S or G; and Xc is Q, K, or E; and
      (vi) NGNGNSQ (SEQ ID NO: 118);
      wherein the polypeptide has at least 70% identity to the catalytic domain of SEQ ID NO: 61.
46. In some embodiments of the recombinant host cell of paragraph 45, wherein the polypeptide comprises a substitution, deletion or addition at a position corresponding to position 102 of the polypeptide of SEQ ID NO: 61.
47. In some embodiments of the recombinant host cell of paragraph 46, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.
48. In some embodiments of the recombinant host cell of any one of paragraphs 45-47, the polypeptide comprises a substitution, deletion or addition at a position corresponding to position 66 of the polypeptide of SEQ ID NO: 61.
49. In some embodiments of the recombinant host cell of paragraph 48, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H.
50. In some embodiments of the recombinant host cell of any one of paragraphs 45-49, wherein the polypeptide comprises SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:119, SEQ ID NO:120, SEQ ID NO:121, SEQ ID NO:122, SEQ ID NO:123, SEQ ID NO:126, SEQ ID NO:129, SEQ ID NO:132, SEQ ID NO:135, SEQ ID NO: 138, SEQ ID NO:140, SEQ ID NO:141, or SEQ ID NO:142.
51. In some embodiments of the recombinant host cell of any one of paragraphs 45-50, which is an ethanologenic microorganism.
52. In some embodiments of the recombinant host cell of paragraph 51, which is a yeast cell.
53. In some embodiments of the recombinant host cell of any one of paragraphs 45-52, wherein said host cell is not Saksenaea vasiformis.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes

### BRIEF DESCRIPTION OF THE DRAWINGS

FIG. 1A-K provide a multiple amino acid sequence alignment of the catalytic domain regions of Mucorales-clade glucoamylases and various reference fungal glucoamylases.

FIG. 2 provides a phylogenetic tree of Mucorales-clade glucoamylases and other fungal glucoamylases.

FIG. 3 provides an alignment of Mucorales-clade GA amino acid sequences (numbered according to SvaGa1 catalytic domain region, SEQ ID NO: 81) across region spanning residues 50 to 70, showing motif 1: 57Y-58Xₐ-59X_{b}-60T-61X-62X-63X_{c}-64X_{d}, wherein X is any amino acid and Xₐ is N or S; X_{b} is T, S, or R; X_{c} is G or N; and X_{d} is D, N, or S.

FIG. 4 provides an alignment of Mucorales-clade GA amino acid sequences (numbered according to SvaGa1 catalytic domain region, SEQ ID NO: 81) across region spanning residues 240 to 260, showing motif 2: 244Xₐ-245X_{b}-246X_{c}-247X_{c}-248A-249A-250N-251X-252X_{d}, wherein X is any amino acid and Xₐ is S or A; X_{b} is T, N, or V; X_{c} is L or I; and X_{d} is A or G.

FIG. 5 provides an alignment of Mucorales-clade GA amino acid sequences (numbered according to SvaGa1 catalytic domain region, SEQ ID NO: 81) across region spanning residues 299 to 315, showing motif 3: 304Xₐ-305G-306X-307G-308N-309X_{b}-310X_{c}, wherein X is any amino acid and Xₐ is N or D; X_{b} is S or G; and X_{c} is Q, K, or E.

FIG. 6A-D provides a multiple amino acid sequence alignment of the catalytic domain regions of additional Mucorales-clade glucoamylases and various reference fungal glucoamylases.

FIG. 7 provides a phylogenetic tree of additional Mucorales-clade glucoamylases and other fungal glucoamylases.

### DETAILED DESCRIPTION

The present disclosure relates to a recombinant host cell, a composition comprising a glucoamylase and methods of saccharifying the starch substrate using the glucoamylase. Moreover, the disclosure also relates to a process of producing fermentation products and a method for increasing starch digestibility in an animal as well as a method of producing a fermented beverage.

### I. DEFINITIONS

Prior to describing the compositions and methods in detail, the following terms and abbreviations are defined.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

The term “glucoamylase (1,4-alpha-D-glucan glucohydrolase, EC 3.2.1.3) activity” is defined herein as an enzyme activity, which catalyzes the release of D-glucose from the non-reducing ends of starch or related oligo- and poly-saccharide molecules.

The term “amino acid sequence” is synonymous with the terms “polypeptide”, “protein” and “peptide” and are used interchangeably. Where such amino acid sequences exhibit activity, they may be referred to as an “enzyme”. The conventional one-letter or three-letter codes for amino acid residues are used, with amino acid sequences being presented in the standard amino-to-carboxy terminal orientation (i.e., N→C).

The term “mature polypeptide” is defined herein as a polypeptide in its final form following translation and any post-translational modifications, such as N-terminal processing, C-terminal truncation, glycosylation, phosphorylation, etc. In one aspect, the predicted mature polypeptide is SEQ ID NO: 61 based on the analysis of SignalP software version 4.0 (Nordahl Petersen et al. (2011) Nature Methods, 8:785-786) and SEQ ID NO: 41 is a signal peptide. In another aspect, the mature polypeptide comprises amino acid position 20-468 of SEQ ID NO:142. In another aspect, the mature polypeptide comprises amino acid position 21-468 of SEQ ID NO:142. In another aspect, the mature polypeptide comprises amino acid position 22-468 of SEQ ID NO:142. In another aspect, the mature polypeptide comprises amino acid position 23-468 of SEQ ID NO:142. In another aspect, the mature polypeptide comprises amino acid position 24-468 of SEQ ID NO:142. In another aspect, the mature polypeptide comprises amino acid position 25-468 of SEQ ID NO:142.

A “signal sequence” or “signal peptide” is a sequence of amino acids attached to the N-terminal portion of a protein, which facilitates the secretion of the protein outside the cell. The mature form of an extracellular protein lacks the signal sequence, which is cleaved off during the secretion process. In some embodiments, SEQ ID NO: 41 is a signal peptide. In other embodiments, the signal peptide comprises amino acid positions 1-20 of SEQ ID NO:142. In other embodiments, the signal peptide comprises amino acid positions 1-21 of SEQ ID NO:142. In other embodiments, the signal peptide comprises amino acid positions 1-22 of SEQ ID NO:142. In other embodiments, the signal peptide comprises amino acid positions 1-23 of SEQ ID NO:142. In other embodiments, the signal peptide comprises amino acid positions 1-24 of SEQ ID NO:142.

The term “nucleic acid” encompasses DNA, RNA, heteroduplexes, and synthetic molecules capable of encoding a polypeptide. Nucleic acids may be single stranded or double stranded, and may be chemically modified. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant nucleotide sequence.

The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

A “synthetic” molecule is produced by in vitro chemical or enzymatic synthesis rather than by an organism.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “expression” refers to the process by which a polypeptide is produced based on a nucleic acid sequence. The process includes both transcription and translation.

The term “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

The term “sequence motif” is a nucleotide or amino-acid sequence pattern that is widespread and has been proven or assumed to have a biological significance. In this invention, the sequence motif is an amino-acid sequence motif identified in the Mucorales-clade glucoamylases.

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

“Percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty: OFF.

The term “homologous sequence” is defined herein as a predicted protein having an E value (or expectancy score) of less than 0.001 in a tfasty search (Pearson, W. R., 1999, in Bioinformatics Methods and Protocols, S. Misener and S. A. Krawetz, ed., pp. 185-219) with the glucoamylase of SEQ ID NO: 61.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

The terms, “wild-type”, “parental” or “reference” with respect to a polypeptide, refer to a naturally-occurring polypeptide that does not include a man-made substitution, insertion, or deletion at one or more amino acid positions. Similarly, the terms “wild-type”, “parental” or “reference” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

A “slurry” is an aqueous mixture containing insoluble starch granules in water.

The term “total sugar content” refers to the total soluble sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

The term “high DS” refers to aqueous starch slurry with a dry solid content greater than 38% (wt/wt).

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4+ (>DP3) denotes polymers with a degree of polymerization of greater than 3.

The term “contacting” refers to the placing of referenced components (including but not limited to enzymes, substrates, and fermenting organisms) in sufficiently close proximity to affect an expect result, such as the enzyme acting on the substrate or the fermenting organism fermenting a substrate.

As used herein, the terms “yeast cells,” “yeast strains,” or simply “yeast” refer to organisms from the Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

An “ethanologenic microorganism” refers to a microorganism with the ability to convert a sugar or other carbohydrates to ethanol.

The term “biochemicals” refers to a metabolite of a microorganism, such as citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, iso-butanol, an amino acid, lysine, itaconic acid, other organic acids, 1,3-propanediol, vitamins, or isoprene or other biomaterial.

The term “pullulanase” also called debranching enzyme (E.C. 3.2.1.41, pullulan 6-glucanohydrolase), is capable of hydrolyzing alpha 1-6 glucosidic linkages in an amylopectin molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −15% to +15% of the numerical value, unless the term is otherwise specifically defined in context.

The following abbreviations/acronyms have the following meanings unless otherwise specified:

| | |
|---|---|
| EC | enzyme commission |
| CAZy | carbohydrate active enzyme |
| w/v | weight/volume |
| w/w | weight/weight |
| v/v | volume/volume |
| wt % | weight percent |
| °C | degrees Centigrade |
| g or gm | gram |
| μg | microgram |
| mg | milligram |
| kg | kilogram |
| μL and μl | microliter |
| mL and ml | milliliter |
| mm | millimeter |
| μm | micrometer |
| mol | mole |
| mmol | millimole |
| M | molar |
| mM | millimolar |
| μM | micromolar |
| nm | nanometer |
| U | unit |
| ppm | parts per million |
| hr and h | hour |
| EtOH | ethanol |

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

The term “comprising” and its cognates are used in their inclusive sense; that is, equivalent to the term “including” and its corresponding cognates. It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. POLYPEPTIDES HAVING GLUCOAMYLASE ACTIVITY

In a first aspect, the present invention relates to polypeptides comprising an amino acid sequence having preferably at least 70%, at least 75%, at least 80%, at least 85%, at least 90%, at least 92%, at least 93%, at least 94%, at least 95%, at least 96%, at least 97%, at least 98%, and even at least 99%, amino acid sequence identity to the polypeptide of SEQ ID NO: 61 or SEQ ID NO:142 and having glucoamylase activity. In another aspect, provided herein are polypeptides comprising an amino acid sequence having preferably at least 70%, at least 75%, at least 80%, at least 85%, at least 90%, at least 92%, at least 93%, at least 94%, at least 95%, at least 96%, at least 97%, at least 98%, and even at least 99%, amino acid sequence identity to a polypeptide comprising amino acid position 20-468 of SEQ ID NO:142, amino acid position 21-468 of SEQ ID NO:142, amino acid position 22-468 of SEQ ID NO:142, amino acid position 23-468 of SEQ ID NO:142, amino acid position 24-468 of SEQ ID NO:142, or amino acid position 25-468 of SEQ ID NO:142.

In some embodiments, the polypeptide comprises an amino acid sequence having at least 70% but less than 100% sequence identity to the polypeptide of SEQ ID NO: 61 or SEQ ID NO:142. In other embodiments, the polypeptide comprises an amino acid sequence having at least 70% but less than 100% sequence identity to the polypeptide comprising amino acid position 20-468 of SEQ ID NO:142, amino acid position 21-468 of SEQ ID NO:142, amino acid position 22-468 of SEQ ID NO:142, amino acid position 23-468 of SEQ ID NO:142, amino acid position 24-468 of SEQ ID NO:142, or amino acid position 25-468 of SEQ ID NO:142. In some embodiments, the polypeptide is non-naturally occurring (i.e. does not occur in nature and is a product of human ingenuity).

In some embodiments, the polypeptides of the present invention are homologous polypeptides comprising amino acid sequences that differ by no more than ten amino acids, no more than nine amino acids, no more than eight amino acids, no more than seven amino acids, no more than six amino acids no more than five amino acids, no more than four amino acids, no more than three amino acids, no more than two amino acids, and even no more than one amino acid from the polypeptide of SEQ ID NO: 61, the polypeptide of SEQ ID NO:142, the polypeptide comprising amino acid position 20-468 of SEQ ID NO:142, the polypeptide comprising amino acid position 21-468 of SEQ ID NO:142, the polypeptide comprising amino acid position 22-468 of SEQ ID NO:142, the polypeptide comprising amino acid position 23-468 of SEQ ID NO:142, the polypeptide comprising amino acid position 24-468 of SEQ ID NO:142, or the polypeptide comprising amino acid position 25-468 of SEQ ID NO:142.

In some embodiments, the polypeptides of the present invention are the catalytic regions comprising amino acids 18 to 449 of SEQ ID NO: 61, predicted by ClustalX Hypertext Transfer Protocol Secure://world wide web.ncbi.nlm.nih.gov/pubmed/17846036.

In some embodiments, the polypeptides of the present invention have pullulan-hydrolyzing activity.

In a second aspect, the present glucoamylases disclosed herein comprise conservative substitution(s) of one or several amino acid residues relative to the amino acid sequence of SEQ ID NO: 61, the polypeptide of SEQ ID NO:142, the polypeptide comprising amino acid position 20-468 of SEQ ID NO:142, the polypeptide comprising amino acid position 21-468 of SEQ ID NO:142, the polypeptide comprising amino acid position 22-468 of SEQ ID NO:142, the polypeptide comprising amino acid position 23-468 of SEQ ID NO:142, the polypeptide comprising amino acid position 24-468 of SEQ ID NO:142, or the polypeptide comprising amino acid position 25-468 of SEQ ID NO:142. Exemplary conservative amino acid substitutions are listed below. Some conservative substitutions (i.e., mutations) can be produced by genetic manipulation while others are produced by introducing synthetic amino acids into a polypeptide by other means.

| For Amino | | |
|---|---|---|
| Acid | Code | Replace with any of |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some embodiments, the polypeptides of the present invention are the variants of the polypeptide of SEQ ID NO: 61, the polypeptide of SEQ ID NO: 142, the polypeptide comprising amino acid position 20-468 of SEQ ID NO: 142, the polypeptide comprising amino acid position 21-468 of SEQ ID NO: 142, the polypeptide comprising amino acid position 22-468 of SEQ ID NO:142, the polypeptide comprising amino acid position 23-468 of SEQ ID NO:142, the polypeptide comprising amino acid position 24-468 of SEQ ID NO:142, or the polypeptide comprising amino acid position 25-468 of SEQ ID NO:142, or a fragment thereof having glucoamylase activity. The variant glucoamylase comprises a deletion, substitution, insertion, or addition of one or a few amino acid residues relative to the amino acid sequence of SEQ ID NO: 61 or SEQ TD NO: 142 or a homologous sequence thereof. In all cases, the expression “one or a few amino acid residues” refers to 10 or less, i.e., 1, 2, 3, 4, 5, 6, 7, 8, 9, or 10, amino acid residues. The amino acid substitutions, deletions and/or insertions of the polypeptide of SEQ ID NO: 61, the polypeptide of SEQ ID NO:142, the polypeptide comprising amino acid position 20-468 of SEQ ID NO:142, the polypeptide comprising amino acid position 21-468 of SEQ ID NO:142, the polypeptide comprising amino acid position 22-468 of SEQ ID NO:142, the polypeptide comprising amino acid position 23-468 of SEQ ID NO:142, the polypeptide comprising amino acid position 24-468 of SEQ ID NO:142, or the polypeptide comprising amino acid position 25-468 of SEQ ID NO:142 can be at most 10, at most 9, at most 8, at most 7, at most 6, at most 5, at most 4, at most 3, at most 2, and even at most 1.

In some embodiments, the variant alteration comprises or consists of a substitution at a position corresponding to position 102 of the polypeptide of SEQ ID NO: 61. In some embodiments, the amino acid at a position corresponding to position 102 of the polypeptide of SEQ ID NO: 61 is substituted with Ala, Arg, Asn, Asp, Cys, Gln, Glu, Gly, His, Leu, Ile, Lys, Met, Phe, Pro, Thr, Trp, Tyr, or Val, preferably with Pro. In some embodiments, the variant alteration comprises or consists of the substitution S102P of the polypeptide of SEQ ID NO: 61. In a further embodiment, the variant comprises or consists of the amino acid sequence of SEQ ID NO:104 or SEQ ID NO:141.

In some embodiments, the variant alteration comprises or consists of a substitution at a position corresponding to position 66 of the polypeptide of SEQ ID NO: 61. In some embodiments, the amino acid at a position corresponding to position 85 of the polypeptide of SEQ ID NO: 61 is substituted with Ala, Arg, Asn, Asp, Cys, Gln, Glu, Gly, His, Leu, Ile, Lys, Met, Phe, Pro, Ser, Thr, Trp, or Tyr. In some embodiments, the variant alteration comprises or consists of the substitution V66A of the polypeptide of SEQ ID NO: 61.

In another embodiment, the variant alteration comprises or consists of a substitution at a position corresponding to positions 66 and position 102 of the polypeptide of SEQ ID NO: 61. In some embodiments, the variant alteration comprises or consists of the substitution V66A and S102P of the polypeptide of SEQ ID NO: 61. In a further embodiment, the variant comprises or consists of the amino acid sequence of SEQ ID NO:140.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

### III. PRODUCTION OF GLUCOAMYLASE

The present glucoamylases can be produced in host cells, for example, by secretion or intracellular expression. A cultured cell material (e.g., a whole-cell broth) comprising a glucoamylase can be obtained following secretion of the glucoamylase into the cell medium. Optionally, the glucoamylase can be isolated from the host cells, or even isolated from the cell broth, depending on the desired purity of the final glucoamylase. A gene encoding a glucoamylase can be cloned and expressed according to methods well known in the art. Suitable host cells include bacterial, fungal (including yeast and filamentous fungi), and plant cells (including algae). Particularly useful host cells include Aspergillus niger, Aspergillus oryzae, Trichoderma reesi, or Myceliophthora thermophila. Other host cells include bacterial cells, e.g., Bacillus subtilis or B. licheniformis, as well as Streptomyces. A suitable yeast host organism can be selected from Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism.

Additionally, the host may express one or more accessory enzymes, proteins, peptides. These may benefit liquefaction, saccharification, fermentation, SSF, and downstream processes. Furthermore, the host cell may produce ethanol and other biochemicals or biomaterials in addition to enzymes used to digest the various feedstock(s). Such host cells may be useful for fermentation or simultaneous saccharification and fermentation processes to reduce or eliminate the need to add enzymes.

### A. Vectors

A DNA construct comprising a nucleic acid encoding a glucoamylase polypeptide can be constructed such that it is suitable to be expressed in a host cell. Because of the known degeneracy in the genetic code, different polynucleotides that encode an identical amino acid sequence can be designed and made with routine skill. It is also known that, depending on the desired host cells, codon optimization may be required prior to attempting expression.

A polynucleotide encoding a glucoamylase polypeptide of the present disclosure can be incorporated into a vector. Vectors can be transferred to a host cell using known transformation techniques, such as those disclosed below.

A suitable vector may be one that can be transformed into and/or replicated within a host cell. For example, a vector comprising a nucleic acid encoding a glucoamylase polypeptide of the present disclosure can be transformed and/or replicated in a bacterial host cell as a means of propagating and amplifying the vector. The vector may also be suitably transformed into an expression host, such that the encoding polynucleotide is expressed as a functional glucoamylase enzyme.

A representative useful vector is pTrex3gM (see, Published US Patent Application 20130323798) and pTTT (see, Published US Patent Application 20110020899), which can be inserted into genome of host. The vectors pTrex3gM and pTTT can both be modified with routine skill such that they comprise and express a polynucleotide encoding a glucoamylase polypeptide of the invention.

An expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the glucoamylase to a host cell organelle such as a peroxisome, or to a particular host cell compartment. For expression under the direction of control sequences, the nucleic acid sequence of the glucoamylase is operably linked to the control sequences in proper manner with respect to expression.

A polynucleotide encoding a glucoamylase polypeptide of the present invention can be operably linked to a promoter, which allows transcription in the host cell. The promoter may be any DNA sequence that shows transcriptional activity in the host cell of choice and may be derived from genes encoding proteins either homologous or heterologous to the host cell. Examples of promoters for directing the transcription of the DNA sequence encoding a glucoamylase, especially in a bacterial host, include the promoter of the lac operon of E. coli, the Streptomyces coelicolor agarase gene dagA or celA promoters, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, and the like.

For transcription in a fungal host, examples of useful promoters include those derived from the gene encoding Aspergillus oryzae TAKA amylase, Rhizomucor miehei aspartic proteinase, Aspergillus niger neutral α-amylase, Aspergillus niger acid stable α-amylase, Aspergillus niger glucoamylase, Rhizomucor miehei lipase, Aspergillus oryzae alkaline protease, Aspergillus oryzae triose phosphate isomerase, Aspergillus nidulans acetamidase and the like. When a gene encoding a glucoamylase is expressed in a bacterial species such as an E. coli, a suitable promoter can be selected, for example, from a bacteriophage promoter including a T7 promoter and a phage lambda promoter. Along these lines, examples of suitable promoters for the expression in a yeast species include but are not limited to the Gal 1 and Gal 10 promoters of Saccharomyces cerevisiae and the Pichia pastoris AOX1 or AOX2 promoters. Expression in filamentous fungal host cells often involves cbh1, which is an endogenous, inducible promoter from T. reesei. See Liu et al. (2008) Acta Biochim. Biophys. Sin (Shanghai) 40(2): 158-65.

The coding sequence can be operably linked to a signal sequence. The DNA encoding the signal sequence may be a DNA sequence naturally associated with the glucoamylase gene of interest to be expressed, or may be from a different genus or species as the glucoamylase. A signal sequence and a promoter sequence comprising a DNA construct or vector can be introduced into a fungal host cell and can be derived from the same source. For example, the signal sequence may be the Trichoderma reesei cbh1 signal sequence, which is operably linked to a cbh1 promoter.

An expression vector may also comprise a suitable transcription terminator and, in eukaryotes, polyadenylation sequences operably linked to the DNA sequence encoding a glucoamylase. Termination and polyadenylation sequences may suitably be derived from the same sources as the promoter.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., Published International PCT Application WO 91/17243.

### B. Transformation and Culture of Host Cells

An isolated cell, either comprising a DNA construct or an expression vector, is advantageously used as a host cell in the recombinant production of a glucoamylase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A glucoamylase expressed by a fungal host cell can be glycosylated, i.e., will comprise a glycosyl moiety. The glycosylation pattern can be the same or different as present in the wild-type glucoamylase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a glucoamylase is stably integrated into a host cell chromosome. Transformants are then selected and purified by known techniques.

### C. Expression and Fermentation

A method of producing a glucoamylase may comprise cultivating a host cell under conditions conducive to the production of the enzyme and recovering the enzyme from the cells and/or culture medium.

The medium used to cultivate the cells may be any conventional medium suitable for growing the host cell and obtaining expression of a glucoamylase polypeptide. Suitable media and media components are available from commercial suppliers or may be prepared according to published recipes (e.g., as described in catalogues of the American Type Culture Collection).

Any of the fermentation methods well known in the art can suitably used to ferment the transformed or the derivative fungal strain as described above. In some embodiments, fungal cells are grown under batch or continuous fermentation conditions.

### D. Methods for Enriching and Purification

Separation and concentration techniques are known in the art and conventional methods can be used to prepare a concentrated solution or broth comprising a glucoamylase polypeptide of the invention.

After fermentation, a fermentation broth is obtained, the microbial cells and various suspended solids, including residual raw fermentation materials, are removed by conventional separation techniques in order to obtain a glucoamylase solution. Filtration, centrifugation, microfiltration, rotary vacuum drum filtration, ultrafiltration, centrifugation followed by ultra-filtration, extraction, or chromatography, or the like, are generally used.

It may at times be desirable to concentrate a solution or broth comprising a glucoamylase polypeptide to optimize recovery. Use of un-concentrated solutions or broth would typically increase incubation time in order to collect the enriched or purified enzyme precipitate.

### IV. COMPOSITIONS

The present invention also relates to compositions comprising a polypeptide and/or a starch substrate. In some embodiments, a polypeptide comprising an amino acid sequence that is at least about 80%, at least about 85%, at least about 90%, at least about 95%, at least about 96%, at least about 97%, at least about 98%, at least about 99%, identical to that of SEQ ID NO: 61 can also be used in the enzyme composition. Preferably, the compositions are formulated to provide desirable characteristics such as low color, low odor and acceptable storage stability at a temperature of about 4-40 °C and a pH of about 3-7.

The composition may comprise a polypeptide of the present invention as the major enzymatic component. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, alpha-glucosidase, beta-glucosidase, beta-amylase, isoamylase, haloperoxidase, invertase, laccase, lipase, mannosidase, oxidase, pectinolytic enzyme, peptidoglutaminase, peroxidase, phytase, polyphenoloxidase, proteolytic enzyme, pullulanase, ribonuclease, transglutaminase, xylanase or a combination thereof, which may be added in effective amounts well known to the person skilled in the art.

The polypeptide compositions may be prepared in accordance with methods known in the art and may be in the form of a liquid or a dry composition. For instance, the compositions comprising the present glucoamylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc, for example, endogenous plant (including algal) enzymes, residual enzymes from a prior processing step, and the like. The polypeptide to be included in the composition may be stabilized in accordance with methods known in the art.

The composition may be cells expressing the polypeptide, including cells capable of producing a product from fermentation. Such cells may be provided in a liquid or in dry form along with suitable stabilizers. Such cells may further express additional polypeptides, such as those mentioned, above.

Examples are given below of preferred uses of the polypeptides or compositions of the invention. The dosage of the polypeptide composition of the invention and other conditions under which the composition is used may be determined on the basis of methods known in the art.

Above composition is suitable for use in liquefaction, saccharification, and/or fermentation process, preferably in starch conversion, especially for producing syrup and fermentation products, such as ethanol. The composition is also suitable for use in animal nutrition and fermented beverage.

### V. USE

The present invention is also directed to use of a polypeptide or composition of the present invention in a liquefaction, a saccharification and/or a fermentation process. The polypeptide or composition may be used in a single process, for example, in a liquefaction process, a saccharification process, or a fermentation process. The polypeptide or composition may also be used in a combination of processes for example in a liquefaction and saccharification process, in a liquefaction and fermentation process, or in a saccharification and fermentation process, preferably in relation to starch conversion.

### A. Saccharification

The liquefied starch may be saccharified into a syrup rich in lower DP (e.g., DP1+DP2) saccharides, using alpha-amylases and glucoamylases, optionally in the presence of another enzyme(s). The exact composition of the products of saccharification depends on the combination of enzymes used, as well as the type of starch processed. Advantageously, the syrup obtainable using the provided glucoamylases may contain a weight percent of DP1 of the total oligosaccharides in the saccharified starch exceeding 90%, e.g., 90%-98% or 95%-97%. The weight percent of DP2 in the saccharified starch may be as low as possible, about less than 3%, e.g., 0-3% or 0-2.8%.

Whereas liquefaction is generally run as a continuous process, saccharification is often conducted as a batch process. Saccharification conditions are dependent upon the nature of the liquefact and type of enzymes available. In some cases, a saccharification process may involve temperatures of about 60-65 °C and a pH of about 4.0-4.5, e.g., pH 4.3. Saccharification may be performed, for example, at a temperature between about 40 °C, about 50 °C, or about 55 °C to about 60 °C or about 65 °C, necessitating cooling of the Liquefact. The pH may also be adjusted as needed. Saccharification is normally conducted in stirred tanks, which may take several hours to fill or empty. Enzymes typically are added either at a fixed ratio to dried solids, as the tanks are filled, or added as a single dose at the commencement of the filling stage. A saccharification reaction to make a syrup typically is run over about 24-72 hours, for example, 24-48 hours. A pre-saccharification can be added before saccharification in a simultaneous saccharification and fermentation (SSF), for typically 40-90 minutes at a temperature between 30-65 °C, typically about 60 °C

### B. Raw Starch Hydrolysis

The present invention provides a use of the glucoamylase of the invention for producing glucoses and the like from raw starch or granular starch. Generally, glucoamylase of the present invention either alone or in the presence of an alpha-amylase can be used in raw starch hydrolysis (RSH) or granular starch hydrolysis (GSH) process for producing desired sugars and fermentation products. The granular starch is solubilized by enzymatic hydrolysis below the gelatinization temperature. Such “low-temperature” systems (known also as “no-cook” or “cold-cook”) have been reported to be able to process higher concentrations of dry solids than conventional systems (e.g., up to 45%).

A “raw starch hydrolysis” process (RSH) differs from conventional starch treatment processes, including sequentially or simultaneously saccharifying and fermenting granular starch at or below the gelatinization temperature of the starch substrate typically in the presence of at least an glucoamylase and/or amylase.

The glucoamylase of the invention may also be used in combination with an enzyme that hydrolyzes only alpha-(1, 6)-glucosidic bonds in molecules comprising at least four glucosyl residues. Preferably, the glucoamylase of the invention is used in combination with pullulanase or isoamylase. The use of isoamylase and pullulanase for debranching of starch, the molecular properties of the enzymes, and the potential use of the enzymes together with glucoamylase is described in G. M. A. van Beynum et al., Starch Conversion Technology, Marcel Dekker, New York, 1985, 101-142.

### C. Fermentation

The soluble starch hydrolysate, particularly a glucose rich syrup, can be fermented by contacting the starch hydrolysate with a fermenting organism typically at a temperature around 32 °C, such as from 30 °C to 35 °C. “Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include yeast, such as Saccharomyces cerevisiae and bacteria, e.g., Zymomonas mobilis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. See Liu et al. (2011) Sheng Wu Gong Cheng Xue Bao 27:1049-56. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. The temperature and pH of the fermentation will depend upon the fermenting organism. Microorganisms that produce other metabolites, such as citric acid and lactic acid, by fermentation are also known in the art. See, e.g., Papagianni (2007) Biotechnol. Adv. 25:244-63; John et al. (2009) Biotechnol. Adv. 27:145-52.

The saccharification and fermentation processes may be carried out as an SSF process. An SSF process can be conducted with fungal cells that express and secrete glucoamylase continuously throughout SSF. The fungal cells expressing glucoamylase also can be the fermenting microorganism, e.g., an ethanologenic microorganism. Ethanol production thus can be carried out using a fungal cell that expresses sufficient glucoamylase so that less or no enzyme has to be added exogenously. The fungal host cell can be selected from an appropriately engineered fungal strains. Fungal host cells that express and secrete other enzymes, in addition to glucoamylase, also can be used. Such cells may express amylase and/or a pullulanase, phytase, alpha-glucosidase, isoamylase, beta-amylase cellulase, xylanase, other hemicellulases, protease, beta-glucosidase, pectinase, esterase, redox enzymes, transferase, or other enzymes. Fermentation may be followed by subsequent recovery of ethanol.

### D. Fermentation Products

The term “fermentation product” means a product produced by a process including a fermentation process using a fermenting organism. Fermentation products contemplated according to the invention include alcohols (e.g., arabinitol, butanol, ethanol, glycerol, methanol, ethylene glycol, propylene glycol, butanediol, glycerin, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); ketones (e.g., acetone); amino acids (e.g., aspartic acid, glutamic acid, glycine, lysine, serine, and threonine); an alkane (e.g., pentane, hexane, heptane, octane, nonane, decane, undecane, and dodecane); a cycloalkane (e.g., cyclopentane, cyclohexane, cycloheptane, and cyclooctane); an alkene (e.g. pentene, hexene, heptene, and octene); gases (e.g., methane, hydrogen (H₂), carbon dioxide (CO₂), and carbon monoxide (CO)); antibiotics (e.g., penicillin and tetracycline); enzymes; vitamins (e.g., riboflavin, B₁₂, beta-carotene); and hormones.

In a preferred aspect the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred fermentation processes used include alcohol fermentation processes, which are well known in the art. Preferred fermentation processes are anaerobic fermentation processes, which are well known in the art.

### E. Brewing

Processes for making beer are well known in the art. See, e.g., Wolfgang Kunze (2004) “Technology Brewing and Malting” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer.

The brewing composition comprising a glucoamylase, in combination with an amylase and optionally a pullulanase and/or isoamylase, may be added to the mash of step (a) above, i.e., during the preparation of the mash. Alternatively, or in addition, the brewing composition may be added to the mash of step (b) above, i.e., during the filtration of the mash. Alternatively, or in addition, the brewing composition may be added to the wort of step (c) above, i.e., during the fermenting of the wort.

### F. Animal Nutrition

The glucoamylases and the compositions described herein can be used as a feed additive for animals to increase starch digestibility. Describe herein is a method for increasing starch digestibility in an animal.

The term “animal” refers to any organism belonging to the kingdom Animalia and includes, without limitation, mammals (excluding humans), non-human animals, domestic animals, livestock, farm animals, zoo animals, breeding stock and the like. For example, there can be mentioned all non-ruminant and ruminant animals. In an embodiment, the animal is a non-ruminant, i.e., a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment, the animal is a ruminant animal including, but not limited to, cattle, young calves, goats, sheep, giraffes, bison, moose, elk, yaks, water buffalo, deer, camels, alpacas, llamas, antelope, pronghorn and nilgai.

The terms “animal feed”, “feed”, “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) byproducts from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

The digestibility of starch in feeds is highly variable and dependent on a number of factors including the physical structure of both the starch and feed matrix. It has been found that starch digestibility in an animal's diet can be improved by the use of at least one glucoamylase as a feed additive.

When used as, or in the preparation of, a feed, such as functional feed, the enzyme or feed additive composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient. For example, there could be mentioned at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

It is also possible that at least one glucoamylase (or an enzyme composition comprising at least one glucoamylase as described herein) described herein can be homogenized to produce a powder. The powder may be mixed with other components known in the art. Optionally, the feedstuff may also contain additional minerals such as, for example, calcium and/or additional vitamins. In some embodiments, the feedstuff is a corn soybean meal mix.

In an alternative preferred embodiment, an enzyme composition comprising at least one glucoamylase can be formulated to granules as described in WO2007/044968 (referred to as TPT granules) or WO1997/016076 or WO1992/012645 incorporated herein by reference. “TPT” means Thermo Protection Technology. When the feed additive composition is formulated into granules, the granules comprise a hydrated barrier salt coated over the protein core. The advantage of such salt coating is improved thermotolerance, improved storage stability and protection against other feed additives otherwise having adverse effect on the enzyme. Preferably, the salt used for the salt coating has a water activity greater than 0.25 or constant humidity greater than 60% at 20 °C. In some embodiments, the salt coating comprises Na₂SO₄.

Alternatively, the composition is in a liquid formulation suitable for consumption preferably such liquid consumption contains one or more of the following: a buffer, salt, sorbitol and/or glycerol.

Any of the glucoamylases described herein for use as a feed additive may be used alone or in combination with at least one direct fed microbial. Categories of DFMs include Bacillus, Lactic Acid Bacteria and Yeasts. Further, any of the glucoamylases described herein for use as a feed additive may be used alone or in combination with at least one essential oil, for example cinnamaldehyde and/or thymol. Still further, any of the glucoamylases described herein for use as a feed additive may be used alone or in combination with at least one additional enzyme. Examples of such enzymes include, without limitation, phytases, xylanases, proteases, amylases, glucanases, or other glucoamylases.

Also disclosed is a method for improving the nutritional value of an animal feed, wherein an effective amount of any of the glucoamylases described herein can be added to animal feed.

The phrase, an “effective amount” as used herein, refers to the amount of an active agent (such as any of the glucoamylase polypeptides disclosed herein) required to confer improved performance on an animal on one or more metrics, either alone or in combination with one or more other active agents (such as, without limitation, one or more additional enzyme(s), one or more DFM(s), one or more essential oils, etc.).

The term “animal performance” as used herein may be determined by any metric such as, without limitation, the feed efficiency and/or weight gain of the animal and/or by the feed conversion ratio and/or by the digestibility of a nutrient in a feed and/or digestible energy or metabolizable energy in a feed and/or by animals' ability to avoid the negative effects of diseases or by the immune response of the subject.

Animal performance characteristics may include but are not limited to: body weight; weight gain; mass; body fat percentage; height; body fat distribution; growth; growth rate; egg size; egg weight; egg mass; egg laying rate; mineral absorption; mineral excretion, mineral retention; bone density; bone strength; feed conversion rate (FCR); average daily feed intake (ADFI); Average daily gain (ADG) retention and/or a secretion of any one or more of copper, sodium, phosphorous, nitrogen and calcium; amino acid retention or absorption; mineralization, bone mineralization carcass yield and carcass quality.

By “improved animal performance on one or more metric” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed comprising the feed additive composition described herein as compared to a feed which does not comprise said feed additive composition.

All references cited herein are herein incorporated by reference in their entirety for all purposes. In order to further illustrate the compositions and methods, and advantages thereof, the following specific examples are given with the understanding that they are illustrative rather than limiting.

### EXAMPLES

### Example 1

### Identification of a Mucorales-Clade Glucoamylase Enzymes

A search for glucoamylase enzymes of the Zygomycetes phylum was performed by scanning annotated protein sequences of the Zygomycetes phylum using dbCAN (Yin et al (2012) “dbCAN: a web resource for automated carbohydrate-active enzyme annoatation: Nucleic Acids Research 40:W4450-451) to identify all GH15 proteins based on CAZY family analysis. A number of genes were identified in the genomes of Mucorales order organisms and the sequences were further analyzed. Genes encoding the Mucorales-clade glucoamylases were identified from the sources listed on Table 1, and are assigned SEQ ID NOs shown on Table 1.

TABLE 1
Sequence source and SEQ ID NOs for Mucorales-clade glucoamylases
evaluated in this study.

| | SEQ | Gene sequence source | |
|---|---|---|---|
| Sample | ID | *[mycocosm.jgi.doe.gov/cgi- | |
| ID | NO | bin/dispGeneModel?db] | Organism |
| SvaGA1 | 1 | *scaffold_1856: 13863-15495, | Saksenaea vasiformis |
| | | protein ID: 4356 | B4078 |
| BciGA1 | 2 | *scaffold_261: 3283-5849, | Backusella circina |
| | | protein ID: 261628 | FSU 941 |
| BciGA2 | 3 | *scaffold_67: 52720-55269, | Backusella circina |
| | | protein ID: 183741 | FSU 941 |
| BpoGA1 | 4 | *scaffold_1: 5688666-5690969, | Benjaminiella |
| | | protein ID: 552608 | poitrasii |
| | | | RSA 903 |
| CcuGA1 | 5 | *scaffold_32: 142308-144412, | Choanephora |
| | | protein ID: 565216 | cucurbitarum |
| | | | NRRL 2744 |
| RstGA1 | 6 | world wide web | Rhizopus stolonifer |
| | | .ncbi.nlm.nih.gov/protein/ | |
| | | RCI05434 | |
| MciGA5 | 7 | world wide web | Mucor circinelloides |
| | | ncbi.nlm.nih.gov/protein/ | f. circinelloides |
| | | EPB90436 | 1006PhL |
| DelGA1 | 8 | *scaffold_14: 156040-158505, | Dichotomocladium |
| | | protein ID: 307234 | elegans RSA 919 |
| FspGA3 | 9 | *scaffold_47: 22656-25504, | Fennellomyces sp. |
| | | protein ID: 631220 | T-0311 |
| GpeGAl | 10 | *scaffold_27: 154515-156499, | Gilbertella persicaria |
| | | protein ID: 572732 | var. persicaria CBS |
| | | | 190.32-T |
| MciGA3 | 11 | *scaffold_04: 2553725- | Mucor circinelloides |
| | | 2555899, protein ID: 156167 | CBS277.49 |
| CumGA1 | 12 | *scaffold_36: 443143-446327, | Circinella umbellata |
| | | protein ID: 486055 | NRRL 1351 |
| McoGA1 | 13 | *scaffold_20: 482726-484980, | Mucor cordense RSA |
| | | protein ID: 382429 | 1222 |
| ParGA1 | 14 | scaffold_11: 462211-464919, | Phascolomyces |
| | | protein ID: 4368711 | articulosus |
| RmiGA1 | 15 | *scaffold_21: 172464-174631, | Rhizopus microsporus |
| | | protein ID: 230588 | var. microsporus |
| | | | ATCC 52813 |
| SfGA2 | 16 | *scaffold_162: 2818-4664, | Spinellus fusiger |
| | | protein ID: 1870629 | NRRL 22323 |
| SraGA1 | 17 | *scaffold_4: 869438-871839, | Syncephalastrum |
| | | protein ID: 545732 | racemosum NRRL |
| | | | 2496 |
| SraGA3 | 18 | *scaffold_9: 632087-633913, | Syncephalastrum |
| | | protein ID: 558396 | racemosum NRRL |
| | | | 2496 |
| TinGA1 | 19 | world wide | Thermomucor |
| | | web.ncbi.nlm.nih.gov/nuccore/ | indicae-seudaticae |
| | | JSYX01000005 | HACC 243 |
| ZmeGA1 | 20 | *scaffold_35: 220637-223894, | Zychaea mexicana |
| | | protein ID: 822592 | RSA 1403 |

The N-terminal signal peptides were predicted by SignalP software version 4.0 (Nordahl Petersen et al. (2011) Nature Methods, 8:785-786). The genes encoding the various Mucorales-clade glucoamylases were codon modified for expression in Trichoderma reesei.

TABLE 2
Sequences of Mucorales-clade glucoamylases evaluated in this study.
SEQ ID NOs for the nucleotide sequences of expression cassettes,
predicted signal peptide and predicted mature polypeptide.

| | SEQ ID Nos | | |
|---|---|---|---|
| | codon modified | | |
| | sequences used as | predicted | predicted mature |
| Sample ID | expression cassettes | signal peptide | protein sequence |
| SvaGA1 | SEQ ID NO: 21 | SEQ ID NO: 41 | SEQ ID NO: 61 |
| BciGA1 | SEQ ID NO: 22 | SEQ ID NO: 42 | SEQ ID NO: 62 |
| BciGA2 | SEQ ID NO: 23 | SEQ ID NO: 43 | SEQ ID NO: 63 |
| BpoGA1 | SEQ ID NO: 24 | SEQ ID NO: 44 | SEQ ID NO: 64 |
| CcuGA1 | SEQ ID NO: 25 | SEQ ID NO: 45 | SEQ ID NO: 65 |
| RstGA1 | SEQ ID NO: 26 | SEQ ID NO: 46 | SEQ ID NO: 66 |
| MciGA5 | SEQ ID NO: 27 | SEQ ID NO: 47 | SEQ ID NO: 67 |
| DelGA1 | SEQ ID NO: 28 | SEQ ID NO: 48 | SEQ ID NO: 68 |
| FspGA3 | SEQ ID NO: 29 | SEQ ID NO: 49 | SEQ ID NO: 69 |
| GpeGA1 | SEQ ID NO: 30 | SEQ ID NO: 50 | SEQ ID NO: 70 |
| MciGA3 | SEQ ID NO: 31 | SEQ ID NO: 51 | SEQ ID NO: 71 |
| CumGA1 | SEQ ID NO: 32 | SEQ ID NO: 52 | SEQ ID NO: 72 |
| McoGA1 | SEQ ID NO: 33 | SEQ ID NO: 53 | SEQ ID NO: 73 |
| ParGA1 | SEQ ID NO: 34 | SEQ ID NO: 54 | SEQ ID NO: 74 |
| RmiGA1 | SEQ ID NO: 35 | SEQ ID NO: 55 | SEQ ID NO: 75 |
| SfuGA2 | SEQ ID NO: 36 | SEQ ID NO: 56 | SEQ ID NO: 76 |
| SraGA1 | SEQ ID NO: 37 | SEQ ID NO: 57 | SEQ ID NO: 77 |
| SraGA3 | SEQ ID NO: 38 | SEQ ID NO: 58 | SEQ ID NO: 78 |
| TinGA1 | SEQ ID NO: 39 | SEQ ID NO: 59 | SEQ ID NO: 79 |
| ZmeGA1 | SEQ ID NO: 40 | SEQ ID NO: 60 | SEQ ID NO: 80 |

### Example 2

Expression of Mucorales-Clade Glucoamylases in Trichoderma reesei

The polynucleotides (codon modified sequences used as expression cassettes) encoding the Mucorales-clade glucoamylases genes (SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40) were synthesized by Generay (Generay Biotech Co., Ltd, Shanghai, China) and inserted into the pGX256 expression vector, a derivative vector from pTTT (see, Published US Patent Application 20110020899).

A polynucleotide encoding a variant of SvaGA1 glucoamylase (SEQ ID NO: 61), where a codon change introduced a mutation at amino acid position 102 of Pro in place of Ser (SvaGA1v2, S102P) was constructed. The expression casette encoding SvaGA1v2 was inserted into the pGX256 (as described above).

All plasmids were transformed into a suitable Trichoderma reesei strain using protoplast transformation (Te'o et al., J. Microbiol. Methods 51:393-99, 2002). The transformants were selected and fermented by the methods described in WO 2016/138315. Supernatants from these cultures were used to confirm the protein expression by SDS-PAGE analysis.

Fungal cell cultures were grown in a defined medium as described by Lv et al (2012) in “Construction of teo vectors for gene expression in Trichoderma reesei”. Plasmids 67:67-71. Clarified culture broth were collected after 96 hours by centrifugation. The Mucorales-clade glucoamylases were purified by methods known in the art. The column chromatography fractions containing the target protein were pooled, concentrated and equilibrated to 20 mM sodium acetate pH 5.0, 150 mM sodium chloride using an Amicon Ultra-15 device with 10 K MWCO. The purified samples were approximately 99% pure (by SDS-PAGE analysis) and were stored in 40% glycerol at −80 °C until use.

### Example 3

### Evaluation of SvaGA1 and SvaGA1v2 Glucoamylases in Saccharification at pH 4.5, 60 °C

The saccharification performance of SvaGA1 and the SvaGA1v2 variant were evaluated at pH 4.5 and 60 °C. A sample of GC126 (a DuPont/IFF product) pre-treated corn starch liquefact (prepared at 38% ds, pH 3.3) was used as a starting substrate. The performance of the glucoamylases was tested at the dosage of 30 μg/gds. The glucoamylase Gloeophyllum trabeum glucoamylase (GtGA) from EXTENDA® XTRA (a Novozymes product) was included for comparison. For this evaluation, the pullulanase OPTIMAX™ L 1000 (a DuPont product) was dosed at 10 μg/gds, and the alpha-amylase Aspergillus kawachii amylase (AkAA, described in WO2013169645, incorporated by reference herein) was dosed at 5 μg/gds for each incubation. The corn starch liquefact substrate and the enzymes (glucoamylase, alpha-amylase and pullulanase) were incubated at pH 4.5, 60 °C for 48 and 65 hours, respectively. All the incubations were quenched by heating at 100 °C for 15 min. Aliquots were removed and diluted 40-fold in 5 mM H₂SO₄ for product analysis by HPLC using an Agilent 1200 series system with a Phenomenex Rezex-RFQ Fast Fruit column (cat #OOD-0223-K0), run at 80 °C. 10 μL samples were loaded on the column and separated with an isocratic gradient of 5 mM H₂SO₄ as the mobile phase at a flow rate of 1.0 mL/min. The oligosaccharide products were detected using a refractive index detector, and the standards were run to determine elution times of each DP(n) sugar of interest (DP3+, DP3, DP2 and DP1). The values shown in Table 3 reflect the peak area percentages of each DP(n) as a fraction of the total DP1 to DP3+. The results of DP1 generation and DP3+ hydrolysis by SvaGA1 and SvaGA1v2 glucoamylases outperformed those of the reference GtGA enzyme at pH 4.5, 60 °C.

TABLE 3
Sugar composition results for glucoamylases incubated with corn
starch liquefact at pH 4.5, 60 °C

| | | Percent (%) Dextrose | | | |
|---|---|---|---|---|---|
| Incubation | | Equivalent (DP) detected | | | |
| time | Sample | DP3+ | DP3 | DP2 | DP1 |
| 48 h | SvaGA1 | 0.4 | 1.2 | 2.7 | 95.7 |
| | SvaGA1v2 | 0.4 | 1.2 | 2.7 | 95.7 |
| | GGA | 0.7 | 1.5 | 3.7 | 94.0 |
| 65 h | SvaGA1 | 0.3 | 1.1 | 2.7 | 95.9 |
| | SvaGA1v2 | 0.3 | 1.0 | 2.9 | 95.8 |
| | GtGA | 0.4 | 1.3 | 2.8 | 95.4 |

### Example 4

### Specific Activities of Mucorales-Clade Glucoamylase Enzymes on Soluble Starch

Glucoamylase specific activity was assayed based on the release of glucose from soluble starch using the coupled glucose oxidase/peroxidase (GOX/HRP) and 2,2′-Azino-bis 3-ethylbenzothiazoline-6-sulfonic acid (ABTS) method (Anal. Biochem. 105 (1980), 389-397). Substrate solutions were prepared by mixing 9 mL of soluble starch (1% in water, w/w) and 1 mL of 0.5 M pH 5.0 sodium acetate buffer in a 15-mL conical tube. Coupled enzyme (GOX/HRP) solution with ABTS was prepared in 50 mM sodium acetate buffer (pH 5.0), with the final concentrations of 2.74 mg/mL ABTS, 0.1 U/mL HRP, and 1 U/mL GOX. Serial dilutions of each glucoamylase sample to be evaluated and a glucose standard were prepared in purified water. Each glucoamylase sample (10 μL) was transferred into a new microtiter plate (Corning 3641) containing 90 μL of substrate solution preincubated at 50 °C for 5 min at 600 rpm. The reactions were carried out at 50 °C for 10 min with shaking (600 rpm) in a thermomixer (Eppendorf), 10 μL of reaction mixtures as well as 10 μL of serial dilutions of glucose standard were quickly transferred to new microtiter plates (Corning 3641), respectively, followed by the addition of 100 μL of ABTS/GOX/HRP solution. Absorbance at 405 nm was immediately measured at 11 seconds intervals for 5 min using a SoftMax Pro plate reader (Molecular Device). The output was the reaction rate, Vo, for each enzyme concentration. Linear regression was used to determine the slope of the plot Vo vs. enzyme dose. The specific activity of each glucoamylase was calculated based on the glucose standard curve using Equation below:

Specific Activity (Unit/mg)=Slope (enzyme)/slope (std)×1000 (1),
where 1 Unit=1 μmol glucose/min.

Using the method described above, specific activities of the Mucorales-clade Glucoamylases and benchmarks was determined. Results are shown in Table 4.

TABLE 4
Specific activity of Mucorales-clade Glucoamylases on soluble starch
after 10-min incubation at pH 5.0, 50 °C

| Sample | Specific activity (U/mg) |
|---|---|
| RmiGA1 | 212.3 |
| SraGA1 | 169.5 |
| BciGA1 | 169.3 |
| SraGA3 | 144.9 |
| BciGA2 | 141.6 |
| MciGA3 | 204.1 |
| McoGA1 | 232.3 |
| DelGA1 | 191.5 |
| GpeGA1 | 233.1 |
| ParGA1 | 144.0 |
| SfuGA2 | 215.7 |
| SvaGA1 | 334.3 |
| TinGA1 | 140.4 |
| AnGA | 191.3 |

### Example 5

### Analysis of Homologous Mucorales-Clade Glucoamylase Sequences

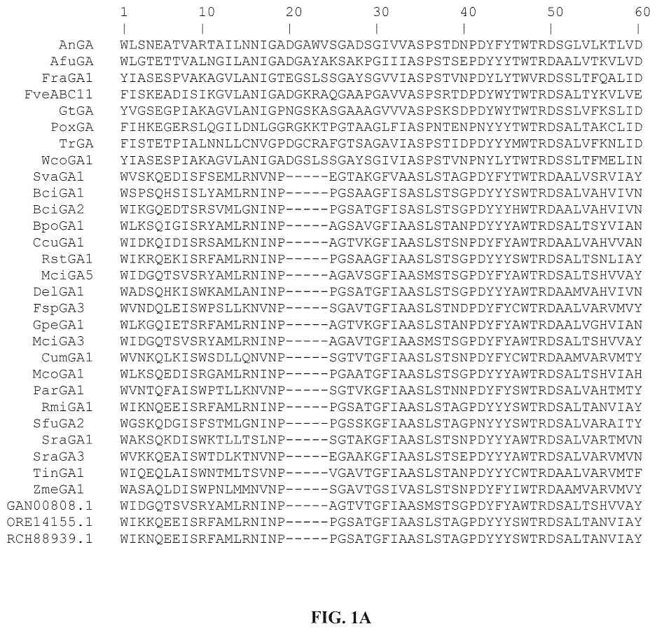

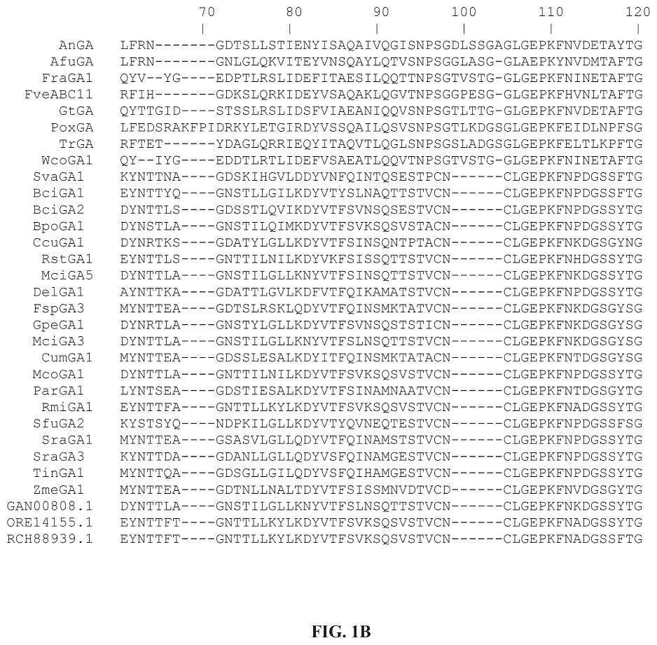

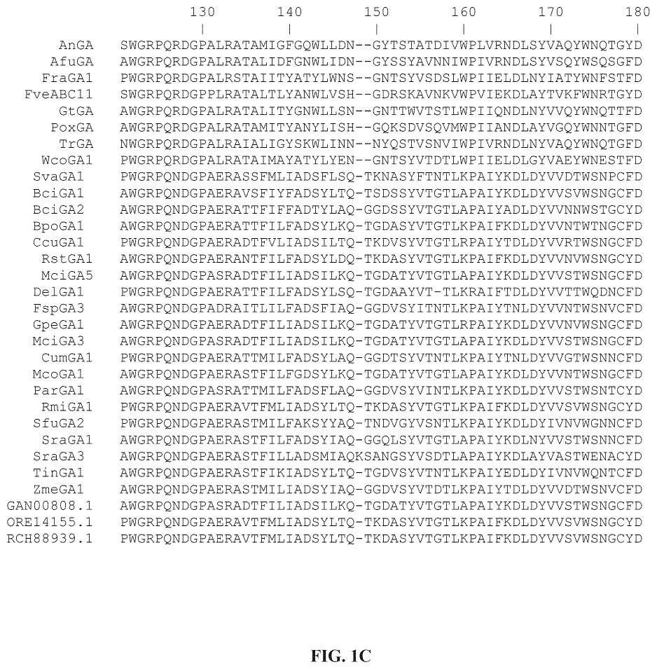

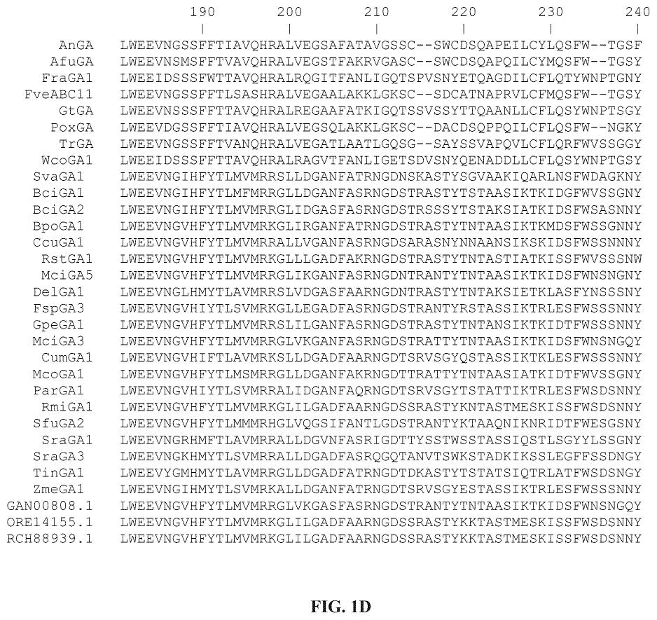

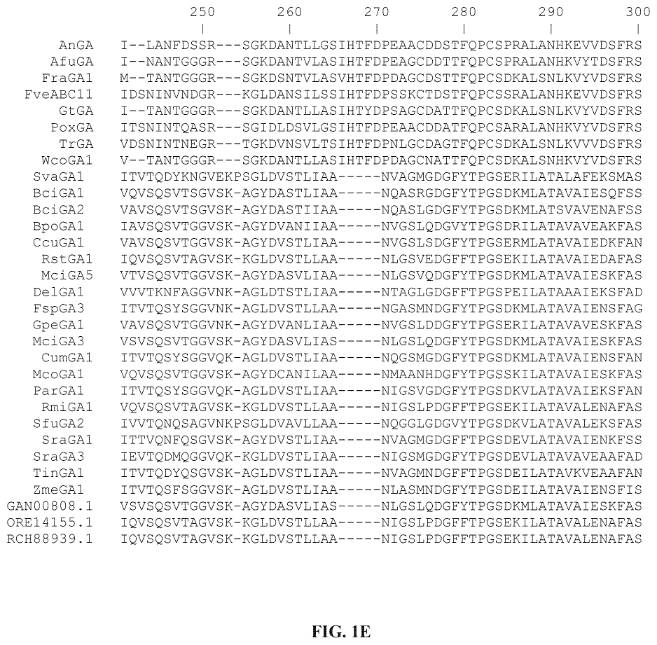

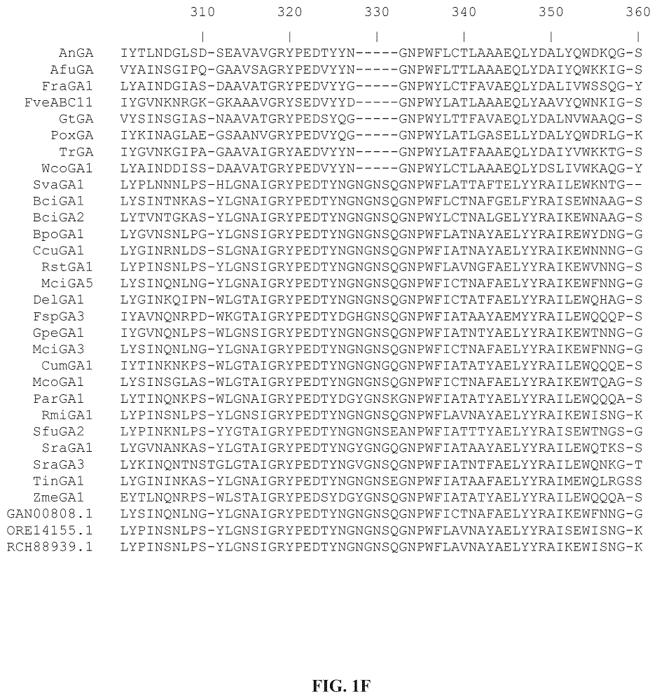

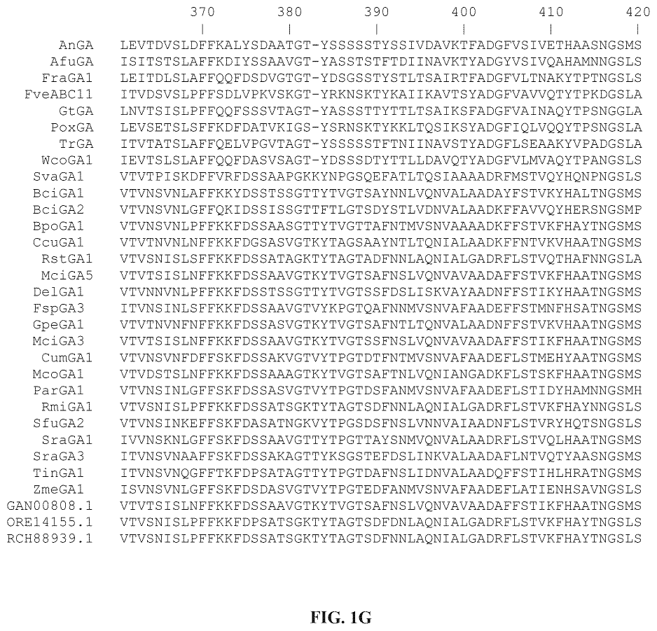

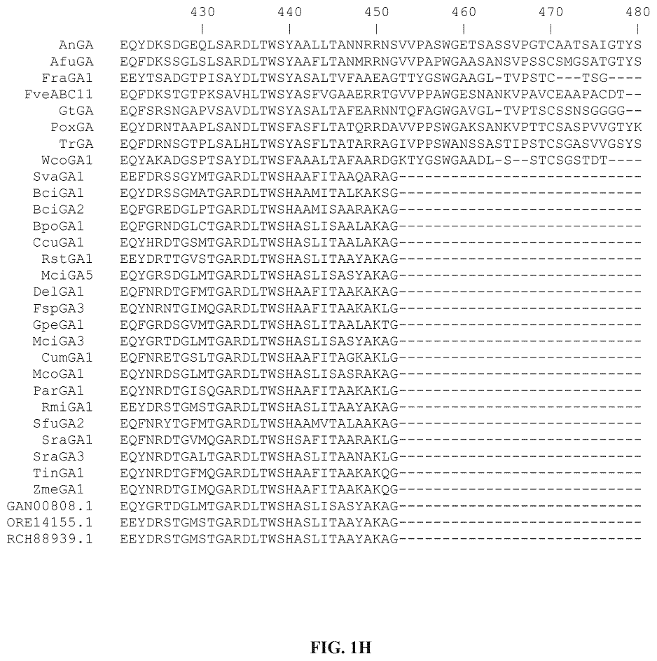

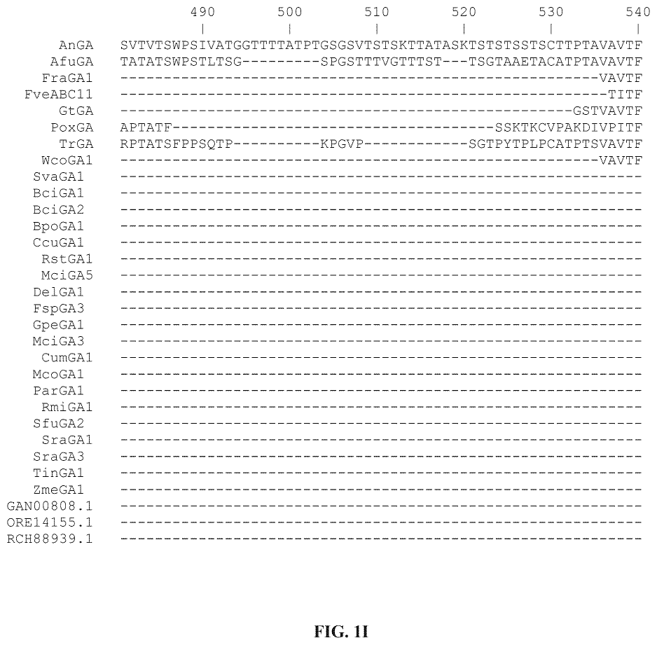

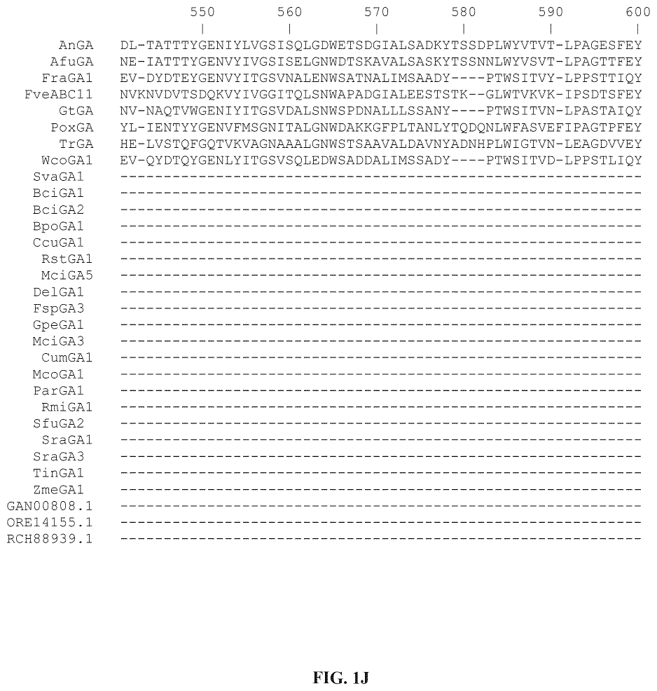

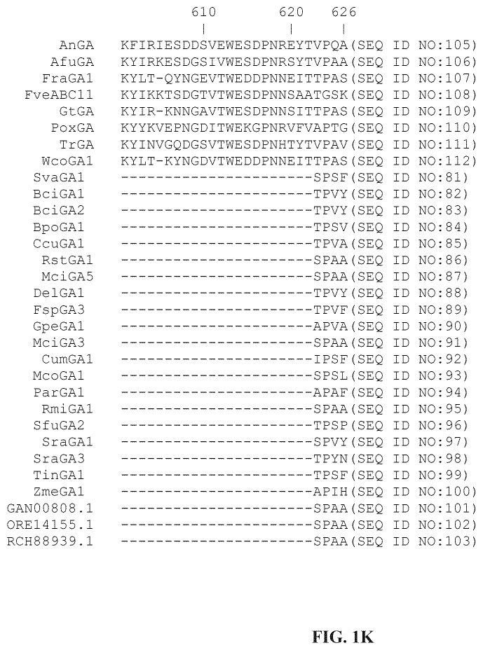

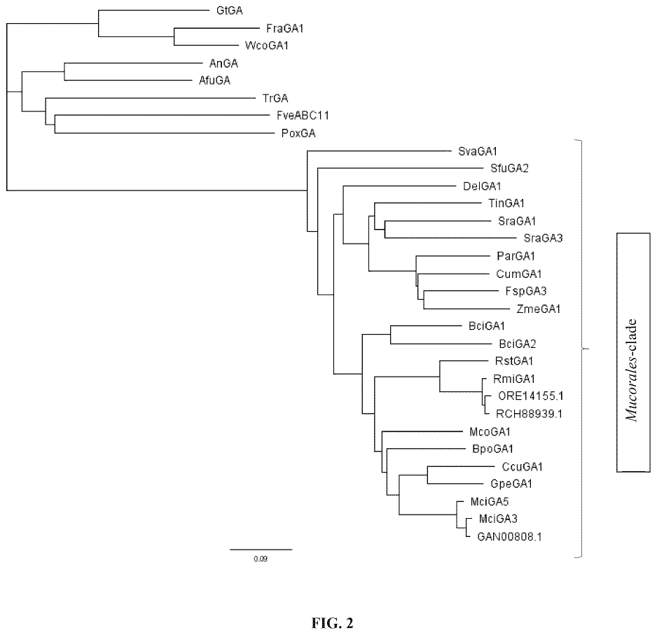

A multiple amino acid sequence alignment was constructed for the regions encompassing the catalytic domain of the Mucorales-clade glucoamylases:SvaGA1 SEQ ID NO:81, BciGA1 SEQ ID NO:82, BciGA2 SEQ ID NO:83, BpoGA1 SEQ ID NO:84, CcuGA1 SEQ ID NO:85, RstGA1 SEQ ID NO:86, MciGA5 SEQ ID NO:87, DelGA1 SEQ ID NO:88, FspGA3 SEQ ID NO:89, GpeGA1 SEQ ID NO:90, MciGA3 SEQ ID NO:91, CumGA1 SEQ ID NO:92, McoGA1 SEQ ID NO:93, ParGA1 SEQ ID NO:94, RmiGA1 SEQ ID NO:95, SfuGA2 SEQ ID NO:96, SraGA1 SEQ ID NO:97, SraGA3 SEQ ID NO:98, TinGA1 SEQ ID NO:99, and ZmeGA1 SEQ ID NO:100. In the case of SvaGa1, the catalytic domain is 432 residues long and spans amino acids 18 to 449 of the predicted mature protein sequence. The above-mentioned region overlaps the previously defined catalytic domains of fungal glucoamylases, based on studies of A. awamori (Aleshin et al, 1994, J. Mol. Bio. 238:575-591) and A. niger (Lee and Paetzel, 2011, Acta Cryst. F67: 188-192) glucoamylase sequences. These sequences were aligned using MUSCLE alignment tool within Geneious 10.2 software with the default parameters. Additional homologous sequences were identified in the public domain: GAN00808.1 SEQ ID NO:101, ORE14155.1 SEQ ID NO:102, and RCH88939.1 SEQ ID NO:103, and their overlapping sequences were also included in this analysis. In addition, the catalytic domains of other glucoamylases, not members of the Mucorales order of fungi, were included in the alignment: Aspergillus niger glucoamylase (AnGA) SEQ ID NO:105, Aspergillus fumigatus glucoamylase (AfuGA) SEQ ID NO:106, Fibroporia radiculosa TFFH 294 glucoamylase (FraGA1) SEQ ID NO:107, Fusarium verticillioides glucoamylase (FveABC11) SEQ ID NO:108, Gloeophyllum trabeum glucoamylase (GtGA) SEQ ID NO:109, Penicillium oxalicum glucoamylase (PoxGA) SEQ ID NO:110, Trichoderma reesei glucoamylase (TrGA) SEQ ID NO:111, and Wolfiporia cocos MD-104 SS10 glucoamylase (WcoGA1) SEQ ID NO:112, in order to identify regions of sequence similarities and differences. The multiple sequence alignment is shown on FIG. 1A-K. A phylogenetic tree was generated using Geneious 10.2 software from the alignment on FIGS. 1A-K and is shown on FIG. 2.

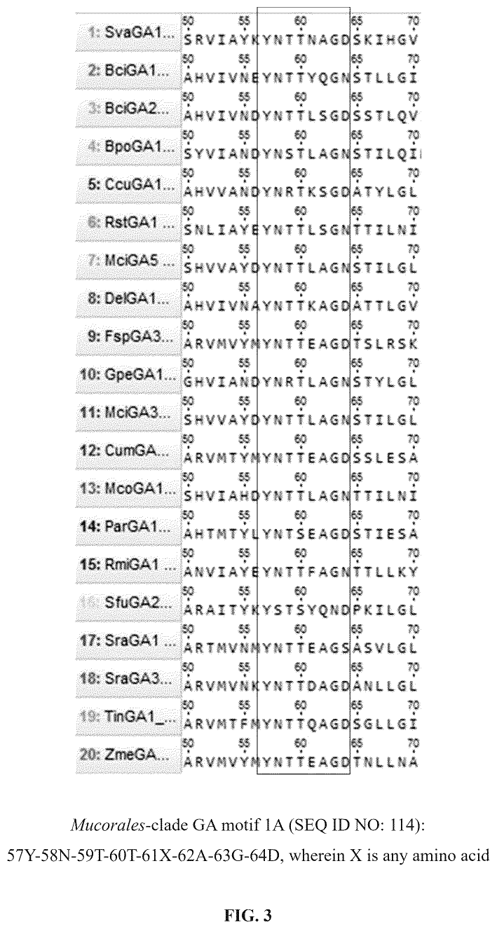

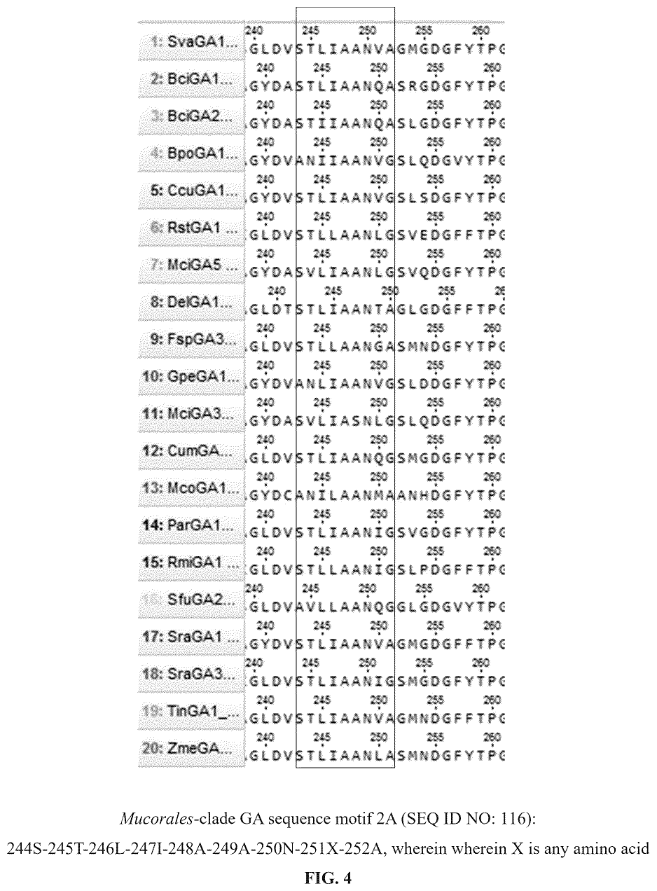

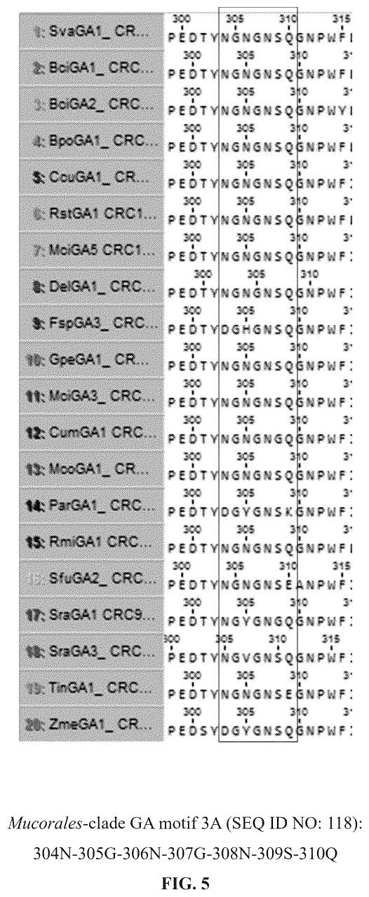

A series of insertions and deletions and high sequence variability regions were observed on the multiple sequence alignment shown on FIG. 1A-FIG. 1K. Several regions of high similarity among the Mucorales-clade glucoamylases become apparent and sequence motifs have been identified. FIGS. 3, 4 and 5 shows alignments of 3 different regions the Mucorales-clade glucoamylases catalytic domains and highlight the sequence motifs in common. FIG. 3 shows the alignment of Mucorales-clade GA amino acid sequences across the region spanning residues 50 to 70 (numbered according to SEQ ID NO: 81), highlighting the Mucorales-clade GA sequence motif 1 (SEQ ID NO: 113): 57Y-58Xₐ-59X_{b}-60T-61X-62X-63X_{c}-64X_{d}, wherein X is any amino acid and Xₐ is N or S; X_{b} is T, S, or R; X_{c} is G or N; and X_{d} is D, N, or S. A further refined motif for this region is the Mucorales-clade GA motif 1A (SEQ ID NO: 114): 57Y-58N-59T-60T-61X-62A-63G-64D, wherein X is any amino acid. FIG. 4 shows the alignment of Mucorales-clade GA amino acid sequences (numbered according to SEQ ID NO: 81) across the region spanning residues 240 to 260, describing the Mucorales-clade GA sequence motif 2 (SEQ ID NO: 115): 244Xₐ-245X_{b}-246X_{c}-247X_{c}-248A-249A-250N-251X-252X_{d}, wherein X is any amino acid and Xₐ is S or A; X_{b} is T, N, or V; X_{c} is L or I; and X_{d} is A or G. A further refined motif for this region is the Mucorales-clade GA sequence motif 2A (SEQ ID NO: 116): 2445-245T-246L-247I-248A-249A-250N-251X-252A, wherein X is any amino acid. FIG. 5 shows the alignment of Mucorales-clade GA amino acid sequences (numbered according to SEQ ID NO: 81) across region spanning residues 299 to 315, describing the Mucorales-clade GA sequence motif 3 (SEQ ID NO: 117): 304Xₐ-305G-306X-307G-308N-309X_{b}-310X_{c}, wherein X is any amino acid and Xₐ is N or D; X_{b} is S or G; and X_{c} is Q, K, or E. A further refined motif for this region is the Mucorales-clade GA motif 3A (SEQ ID NO: 118): 304N-305G-306N-307G-308N-309S-310Q.

### Example 6

### Thermostability Evaluation of Glucoamylases

### Stability Comparison at 60 °C

The thermostability of SvaGA1v2 and SvaGA1v3 was compared with preincubations of the enzyme samples (20 ppm) at 60 °C for 10 min. The preincubation at 4 °C for 10 min was included and set as 100% activity of each glucoamylase sample. The residual activity of the glucoamylase after preincbuation was then measured using the same method as described in Example 4 except the pH was 4.5 and the incubation temperature was 60 °C. As shown in Table 5, SvaGA1v3 retained 55% of its activity under these conditions.

TABLE 5
SvaGA1v2 and SvaGA1v3 stability preincubated at 60 °C for 10 min
followed by residual activity measurement 60 °C for 10 min at pH 4.5

| Sample | 4 °C | 60 °C |
|---|---|---|
| SvaGA1v2 | 100% | 13% |
| SvaGA1v3 | 100% | 55% |

### Tm Measurement Using DSC

Differential scanning calorimetry (DSC) measurements were carried out using an ultrasensitive MicroCal™ VP-Capillary DSC System (GE healthcare). Purified SvaGA1, SvaGA1v2 and SvaGA1v3 were diluted to a final concentration of 0.4 mg/mL in 100 mM pH 4.5 sodium acetate buffer. 400 μL of the enzyme solution, as well as a reference containing an identical amount of enzyme-free buffer, were added to a 96-well plate. The plate was then placed in the thermally controlled autosampler compartment kept at 10 °C. The enzyme sample and the buffer reference were scanned respectively from 20 to 100 °C at a scan rate of 2 °C per minute. Tm was determined as the temperature at the peak maximum of the transition from the folded to unfolded state. Maximum variation in the Tm was ±0.2 °C. The ORIGIN software package (MicroCal, GE Healthcare) was used for baseline subtraction and graph presentation of the data. The DSC result in Table 6 shows that the Tm of SvaGA1v3 is 3 degrees higher than that of SvaGA1v2.

TABLE 6
Tm measurement of SvaGA1v2 and
SvaGA1v3 using DSC.

| Sample | Tm (°C) |
|---|---|
| SvaGA1 | 62 |
| SvaGA1v2 | 64 |
| SvaGA1v3 | 67 |

### Example 7

### Saccharification Activity Determination

The saccharification performance of SvaGA1v2 and SvaGA1v3 were evaluated at pH 4.5 and 60, 62, 65 °C, respectively. All the incubation conditions were the same as described in Example 3, with the exception that the corn starch liquefact was purchased from Cargill. The pullulanase OPTIMAX™ L 1000 (a DuPont product) was dosed at 4 μg/gds, and the alpha-amylase Aspergillus terreus amylase (AtAA, described in, for example, International Patent Application Publication Nos. WO2017112635A1 and WO2014099415, incorporated by reference herein) was dosed at 1 μg/gds. The values shown in Table 7 reflect the peak area percentages of each DP(n) as a fraction of the total DP1 to DP3+. The DP1 generation and DP3+ hydrolysis by SvaGA1v3 indicate superior performance when compared to the reference GtGA enzyme under all the selected conditions.

TABLE 7
Sugar composition results for glucoamylases incubated with corn
starch liquefact at pH 4.5, 60 °C, 62 °C, and 65 °C, for 24 h, 48 h,
and 65 h, respectively.

| | Incubation temp. | Incubation time | | | | |
|---|---|---|---|---|---|---|
| GA sample | (°C) | (h) | DP1 % | DP2 % | DP3 % | DP3+ % |
| SvaGA1v2 | 60 | 24 | 94.5 | 2.6 | 0.9 | 1.9 |
| | | 48 | 96.7 | 2.2 | 0.7 | 0.4 |
| | | 65 | 96.5 | 2.4 | 0.7 | 0.4 |
| | 62 | 24 | 94.6 | 3.0 | 1.0 | 1.5 |
| | | 48 | 96.7 | 2.1 | 0.8 | 0.4 |
| | | 65 | 96.6 | 2.2 | 0.8 | 0.4 |
| | 65 | 24 | 88.5 | 6.2 | 1.0 | 4.3 |
| | | 48 | 91.1 | 5.8 | 1.1 | 2.0 |
| | | 65 | 92.6 | 4.8 | 1.1 | 1.5 |
| SvaGA1v3 | 60 | 24 | 92.9 | 3.4 | 0.9 | 2.7 |
| | | 48 | 96.7 | 2.2 | 0.7 | 0.3 |
| | | 65 | 96.6 | 2.5 | 0.6 | 0.3 |
| | 62 | 24 | 93.8 | 3.1 | 0.9 | 2.2 |
| | | 48 | 96.7 | 2.2 | 0.7 | 0.3 |
| | | 65 | 96.6 | 2.5 | 0.6 | 0.3 |
| | 65 | 24 | 92.9 | 3.4 | 1.0 | 2.7 |
| | | 48 | 96.3 | 2.2 | 0.8 | 0.6 |
| | | 65 | 96.5 | 2.2 | 0.8 | 0.5 |
| GtGA | 60 | 24 | 91.3 | 3.7 | 1.0 | 4.0 |
| | | 48 | 96.3 | 2.2 | 0.8 | 0.7 |
| | | 65 | 96.6 | 2.3 | 0.6 | 0.5 |
| | 62 | 24 | 92.2 | 3.4 | 1.0 | 3.4 |
| | | 48 | 96.3 | 2.2 | 0.7 | 0.8 |
| | | 65 | 96.3 | 2.5 | 0.7 | 0.5 |
| | 65 | 24 | 92.6 | 3.1 | 0.9 | 3.4 |
| | | 48 | 96.1 | 2.3 | 0.7 | 0.9 |
| | | 65 | 96.2 | 2.5 | 0.6 | 0.7 |

### Example 8

### Identification of Additional Homologous Mucorales-Clade Glucoamylases

A number of genes were identified in the genomes of Mucorales order organisms and the sequences were further analyzed. Genes encoding the Mucorales-clade glucoamylases were identified from the sources listed on Table 8, and are assigned the SEQ ID NOs shown on Table 9.

TABLE 8
Sequence source and SEQ ID NOs for Mucorales-clade glucoamylases
evaluated in this study.

| | SEQ | Gene sequence source | |
|---|---|---|---|
| Sample | ID | *[://world wide | |
| ID | NO | web.ncbi.nlm.nih.gov/] | Organism |
| SobGA1 | 119 | *nuccore/JNEV01000736 | Saksenaea |
| | | | oblongispora |
| | | | B3353 |
| AosGA3 | 120 | *protein/KAF7727643 | Apophysomyces |
| | | | ossiformis |
| | | | NRRL A-21654 |
| AelGA1 | 121 | *nuccore/JNDQ01001334.1 | Apophysomyces |
| | | | elegans B7760 |
| AvaGA1 | 122 | *nuccore/MZZL01000409.1 | Apophysomyces |
| | | | variabilis |
| | | | NCCPF 102052 |
| AtrGA1 | 123 | *nuccore/JNDP01001364.1 | Apophysomyces |
| | | | trapeziformis |
| | | | B9324 |

The N-terminal signal peptides were predicted by SignalP software version 4.0 (Nordahl Petersen et al. (2011) Nature Methods, 8:785-786). The genes encoding the various Mucorales-clade glucoamylases were codon modified for expression in Trichoderma reesei. Based at least in part on this analysis, a new variant of SvaGA1, named SvaGA1v3, was made and assigned SEQ ID NO: 140 as showed in Table 9.

TABLE 9
Sequences of additional Mucorales-clade glucoamylases evaluated in
this study. SEQ ID NOs for the nucleotide sequences of expression
cassettes, predicted signal peptide and predicted mature polypeptide.

| | SEQ ID Nos | | |
|---|---|---|---|
| | codon modified | | |
| | sequences used as | predicted signal | predicted mature |
| Sample ID | expression cassettes | peptide | protein sequence |
| SobGA1 | SEQ ID NO: 124 | SEQ ID NO: 125 | SEQ ID NO: 126 |
| AosGA3 | SEQ ID NO: 127 | SEQ ID NO: 128 | SEQ ID NO: 129 |
| AelGA1 | SEQ ID NO: 130 | SEQ ID NO: 131 | SEQ ID NO: 132 |
| AvaGA1 | SEQ ID NO: 133 | SEQ ID NO: 134 | SEQ ID NO: 135 |
| AtrGA1 | SEQ ID NO: 136 | SEQ ID NO: 137 | SEQ ID NO: 138 |
| SvaGA1v3 | SEQ ID NO: 139 | SEQ ID NO: 41 | SEQ ID NO: 140 |

### Example 9

Expression of Mucorales-Clade Glucoamylases in Trichoderma reesei

The polynucleotides (codon modified sequences used as expression cassettes) encoding the Mucorales-clade glucoamylases genes (SEQ ID NO: 124, SEQ ID NO: 127, SEQ ID NO: 130, SEQ ID NO: 133, SEQ ID NO: 136) were synthesized by Generay (Generay Biotech Co., Ltd, Shanghai, China) and inserted into the pGX256 expression vector, a derivative vector from pTTT (see, Published US Patent Application 20110020899).

All plasmids were transformed into a suitable Trichoderma reesei strain using protoplast transformation (Te'o et al., J. Microbiol. Methods 51:393-99, 2002). The transformants were selected and fermented by the methods described in WO 2016/138315. Supernatants from these cultures were used to confirm the protein expression by SDS-PAGE analysis.

Fungal cell cultures were grown in a defined medium as described by Lv et al (2012) in “Construction of teo vectors for gene expression in Trichoderma reesei”. Plasmids 67:67-71. Clarified culture broth were collected after 96 hours by centrifugation. The Mucorales-clade glucoamylases were purified by methods known in the art. The column chromatography fractions containing the target protein were pooled, concentrated and equilibrated to 20 mM sodium acetate pH 5.0, 150 mM sodium chloride using an Amicon Ultra-15 device with 10 K MWCO. The purified samples were approximately 99% pure (by SDS-PAGE analysis) and were stored in 40% glycerol at −80 °C until use.

### Example 10

### Evaluation of Additional Homologous Mucorales-Clade Glucoamylases in Saccharification

The saccharification performance of additional homologous Mucorales-clade glucoamylases were evaluated at pH 4.5, 60 °C, 62 °C, and 65 °C, respectively, for 48 h. All the incubation conditions were the same as described in Example 7, except that the glucoamylase samples were dosed at 25 μg/gds. The values shown in Table 10 reflect the peak area percentages of each DP(n) as a fraction of the total DP1 to DP3+. The results of DP1 generation and DP3+ hydrolysis by the Mucorales-clade glucoamylases indicate superior performance when compared to the reference GtGA enzyme when evaluated at pH 4.5, 60 °C for 48 h. When the incubation temperature was increased to 62 °C, all the Mucorales-clade GAs also showed better saccharification performance than GtGA enzyme. AtrGA1 could maintain its superior performance when the incubation temperature was raised to 65 °C while GtGA did not.

TABLE 10
Sugar composition results for glucoamylases incubated with corn starch
liquefact at pH 4.5, 60, 62, and 65 °C, respectively, for 48 h.

| Incubation | | | | | |
|---|---|---|---|---|---|
| temp. (°C) | Sample | DP1 % | DP2 % | DP3 % | DP3+ % |
| 60 | AosGA3 | 96.6 | 2.3 | 0.7 | 0.4 |
| | AelGA1 | 96.6 | 2.2 | 0.7 | 0.4 |
| | AvaGA1 | 96.6 | 2.2 | 0.8 | 0.4 |
| | AtrGA1 | 96.7 | 2.2 | 0.7 | 0.4 |
| | SvaGA1 | 96.5 | 2.0 | 0.8 | 0.6 |
| | GtGA | 95.9 | 7 7 | 0.8 | 1.0 |
| 62 | AosGA3 | 96.5 | 2.3 | 0.7 | 0.4 |
| | AelGA1 | 96.4 | 2.3 | 0.8 | 0.5 |
| | AvaGA1 | 96.5 | 2.3 | 0.8 | 0.4 |
| | AtrGA1 | 96.6 | 2.3 | 0.7 | 0.4 |
| | SvaGA1 | 95.3 | 2 9 | 1.0 | 0.9 |
| | GtGA | 96.3 | 2.1 | 0.8 | 0.8 |
| 65 | AosGA3 | 95.7 | 2.6 | 0.9 | 0.8 |
| | AelGA1 | 88.1 | 7.6 | 1.4 | 2.9 |
| | AvaGA1 | 95.0 | 3.0 | 1.0 | 1.0 |
| | AtrGA1 | 96.4 | 2.3 | 0.7 | 0.5 |
| | SvaGA1 | 77.1 | 13.1 | 3.0 | 6.7 |
| | GtGA | 95.8 | 2 2 | 0.8 | 1.1 |

### Example 11

### Sequence Analysis of Additional Homologous Mucorales-Clade Glucoamylase Sequences

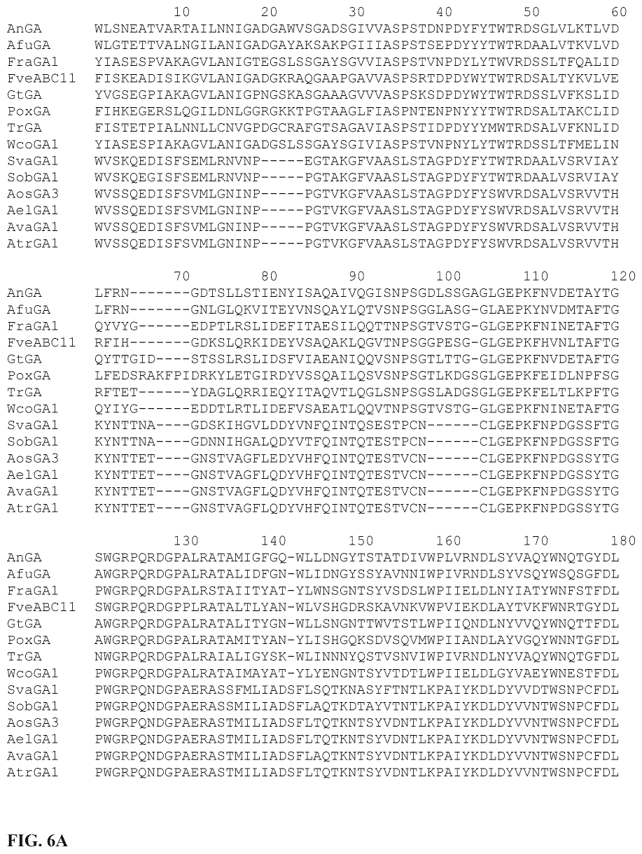

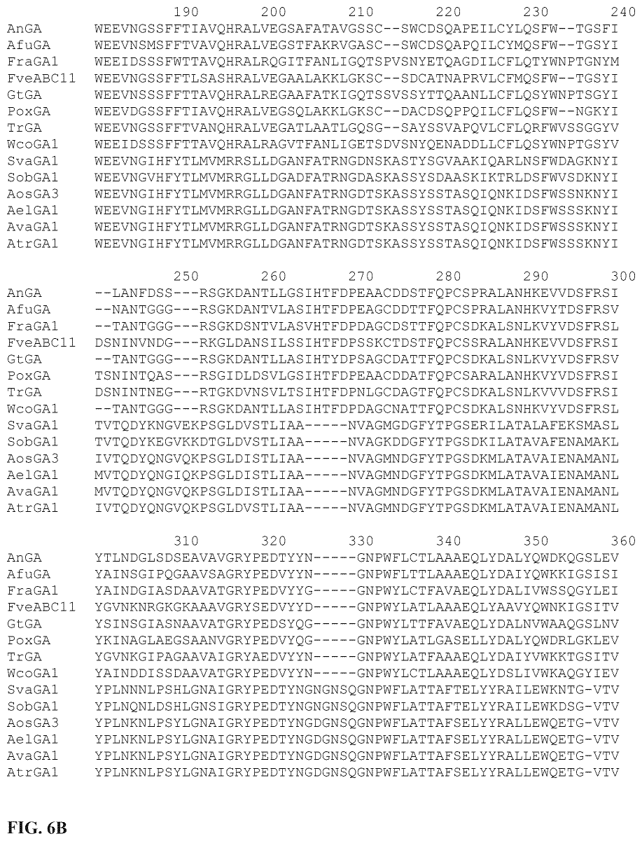

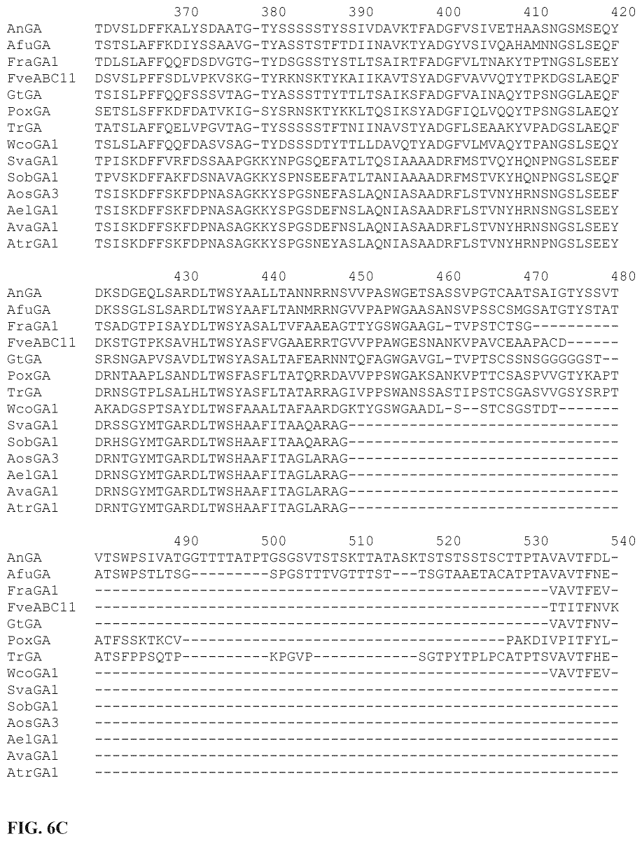

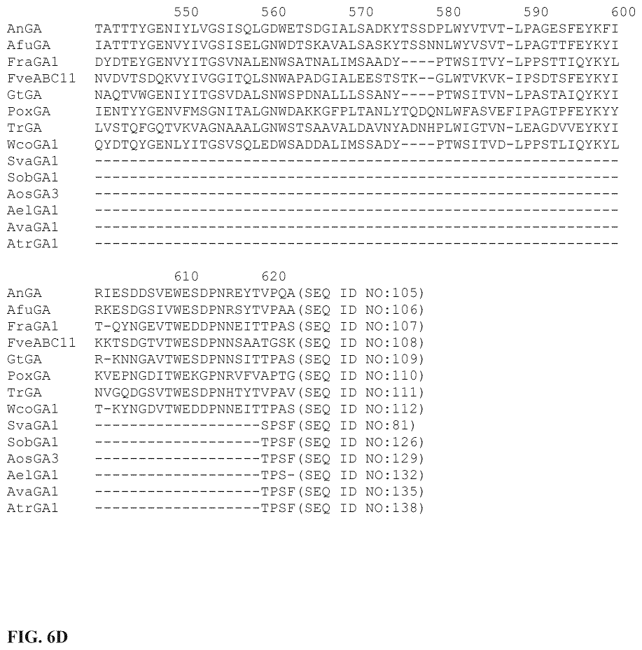

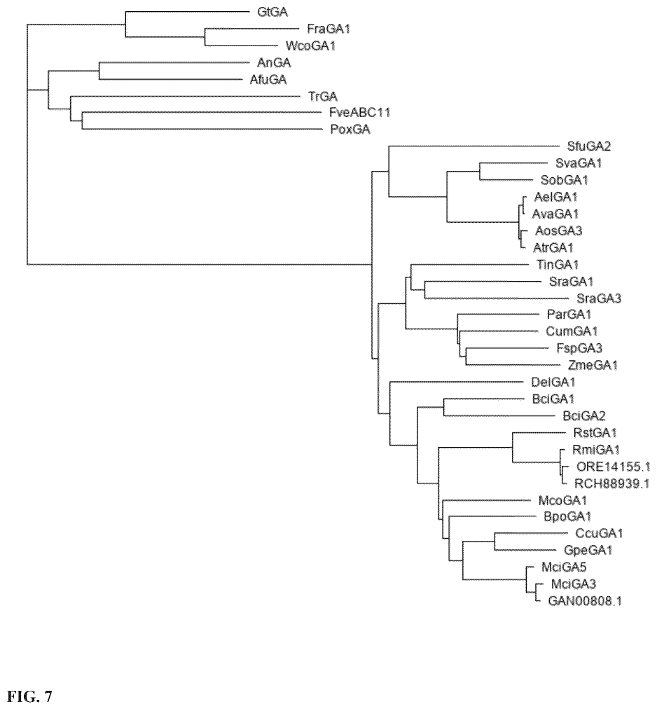

A multiple amino acid sequence alignment was constructed for the regions encompassing the catalytic domain of the Mucorales-clade glucoamylases: SvaGA1 SEQ ID NO:81, SobGA1 SEQ ID NO:119, AosGA3 SEQ ID NO:120, AelGA1 SEQ ID NO:121, AvaGA1 SEQ ID NO:122, and AtrGA1 SEQ ID NO:123 as described in Example 5. These sequences were aligned using MUSCLE alignment tool within Geneious 10.2 software with the default parameters. In addition, the catalytic domains of other glucoamylases, not members of the Mucorales order of fungi, were included in the alignment: Aspergillus niger glucoamylase (AnGA) SEQ ID NO:105, Aspergillus fumigatus glucoamylase (AfuGA) SEQ ID NO:106, Fibroporia radiculosa TFFH 294 glucoamylase (FraGA1) SEQ ID NO:107, Fusarium verticilihoides glucoamylase (FveABC11) SEQ ID NO:108, Gloeophyllum trabeum glucoamylase (GtGA) SEQ ID NO:109, Penicillium oxalicum glucoamylase (PoxGA) SEQ ID NO:110, Trichoderma reesei glucoamylase (TrGA) SEQ ID NO:111, and Wolfiporia cocos MD-104 SS10 glucoamylase (WcoGA1) SEQ ID NO:112, in order to identify regions of sequence similarities and differences. The multiple sequence alignment is shown on FIG. 6A-D. The additional new homologs (SobGA1, AosGA3, AelGA1, AvaGA1, AtrGA1) fall within the motifs outlined in sequences SEQ ID NO:113, SEQ ID NO:115, and SEQ ID NO:117. A phylogenetic tree was generated using Geneious 10.2 software from the alignment of the following sequences: SvaGA1 SEQ ID NO:81, BciGA1 SEQ ID NO:82, BciGA2 SEQ ID NO:83, BpoGA1 SEQ ID NO:84, CcuGA1 SEQ ID NO:85, RstGA1 SEQ ID NO:86, MciGA5 SEQ ID NO:87, DelGA1 SEQ ID NO:88, FspGA3 SEQ ID NO:89, GpeGA1 SEQ ID NO:90, MciGA3 SEQ ID NO:91, CumGA1 SEQ ID NO:92, McoGA1 SEQ ID NO:93, ParGA1 SEQ ID NO:94, RmiGA1 SEQ ID NO:95, SfuGA2 SEQ ID NO:96, SraGA1 SEQ ID NO:97, SraGA3 SEQ ID NO:98, TinGA1 SEQ ID NO:99, ZmeGA1 SEQ ID NO:100, GAN00808.1 SEQ ID NO:101, ORE14155.1 SEQ ID NO:102, and RCH88939.1 SEQ ID NO:103, Aspergillus niger glucoamylase (AnGA) SEQ ID NO:105, Aspergillus fumigatus glucoamylase (AfuGA) SEQ ID NO:106, Fibroporia radiculosa TFFH 294 glucoamylase (FraGA1) SEQ ID NO:107, Fusarium verticillioides glucoamylase (FveABC11) SEQ ID NO:108, Gloeophyllum trabeum glucoamylase (GtGA) SEQ ID NO:109, Penicillium oxalicum glucoamylase (PoxGA) SEQ ID NO:110, Trichoderma reesei glucoamylase (TrGA) SEQ ID NO:111, and Wolfiporia cocos MD-104 SS10 glucoamylase (WcoGA1) SEQ ID NO:112, SobGA1 SEQ ID NO:119, AosGA3 SEQ ID NO:120, AelGA1 SEQ ID NO:121, AvaGA1 SEQ ID NO:122, and AtrGA1 SEQ ID NO:123, and is shown on FIG. 7.

What is claimed is:

1-44. (canceled)

45. A glucoamylase selected from the group consisting of:
a) a polypeptide having an amino acid sequence at least 90% identical to SEQ ID NO: 61 or SEQ ID NO:142;
b) a polypeptide having at least 90% identity to SEQ ID NO: 81; and
c) a polypeptide comprising one or more signature motifs selected from the group consisting of:
(i) YXaXbTXXXcXd (SEQ ID NO: 113), wherein X is any amino acid and Xa is N or S; Xb is T, S, or R; Xc is G or N; and Xd is D, N, or S;
(ii) YNTTXAGD (SEQ ID NO: 114), wherein X is any amino acid;
(iii) XaXbXcXcAANXXd (SEQ ID NO: 115), wherein X is any amino acid and Xa is S or A; Xb is T, N, or V; Xc is L or I; and Xd is A or G;
(iv) STLIAANXA (SEQ ID NO: 116), wherein wherein X is any amino acid;
(v) XaGXGNXbXc (SEQ ID NO: 117), wherein X is any amino acid and Xa is N or D; Xb is S or G; and Xc is Q, K, or E; and
(vi) NGNGNSQ (SEQ ID NO: 118);
wherein the polypeptide has at least 90% identity to the catalytic domain of SEQ ID NO: 61,
wherein the polypeptide comprises a substitution, deletion or addition at a position corresponding to position 102 of the polypeptide of SEQ ID NO: 61 and wherein the polypeptide comprises a substitution, deletion or addition at a position corresponding to position 66 of the polypeptide of SEQ ID NO: 61.

46. (canceled)

47. The glucoamylase of claim 45, wherein the polypeptide comprises a substitution selected from the group consisting of S102P, S102G, S102A, S102V, S102L, S1021, S102F, S102Y, S102W, S102S, S102T, S102C, S102M, S102N, S102Q, S102D, S102E, S102K, S102R, and S102H.

48. (canceled)

49. The glucoamylase of claim 45, wherein the polypeptide comprises a substitution selected from the group consisting of V66P, VS66G, V66A, V66L, V66I, V66F, V66Y, V66W, V66S, V66T, V66C, V66M, V66N, V66Q, V66D, V66E, V66K, V66R, and V66H.

50. The glucoamylase of claim 45, wherein the polypeptide comprises SEQ ID NO:61, or SEQ ID NO:142.

51-53. (canceled)

54. A method for saccharifying a starch substrate, comprising contacting the starch substrate with the glucoamylase of claim 45.

55. The method of claim 54, wherein the starch substrate is about 15% to 65%, 15% to 60% or 15% to 35% dry solid (DS).

56. The method of claim 54, wherein the starch substrate comprises liquefied starch, gelatinized starch, or granular starch.

57. The method of claim 54, further comprising adding a hexokinase, a xylanase, a glucose isomerase, a xylose isomerase, a phosphatase, a phytase, a pullulanase, a beta-amylase, an alpha-amylase, a protease, a cellulase, a hemicellulase, a lipase, a cutinase, a trehalase, an isoamylase, a redox enzyme, an esterase, a transferase, a pectinase, a hydrolase, an alpha-glucosidase, a beta-glucosidase, or a combination thereof to the starch substrate.

58. The method of claim 54, wherein saccharifying the starch substrate results in a high glucose syrup comprising an amount of glucose selected from the group consisting of at least 95.5% glucose, at least 95.6% glucose, at least 95.7% glucose, at least 95.8% glucose, at least 95.9% glucose, at least 96% glucose, at least 96.1% glucose, at least 96.2% glucose, at least 96.3% glucose, at least 96.4% glucose, at least 96.5% glucose and at least 97% glucose.

59. The method of claim 54, further comprising fermenting the high glucose syrup to an end product.

60. The method of claim 59, wherein saccharifying and fermenting are carried out as a simultaneous saccharification and fermentation (SSF) process.

61. The method of claim 59, wherein the end product is an alcohol, optionally, ethanol.

62. The method of claim 59, wherein the end product is a biochemical selected from the group consisting of an amino acid, an organic acid, citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, lysine, itaconic acid, 1,3-propanediol, biodiesel, and isoprene.

63. A process of producing a fermentation product from a starch substrate comprising the steps of:
1) liquefying the starch substrate;
2) saccharifying the liquefied starch substrate; and
3) fermenting with a fermenting organism;
wherein step 2) is carried out using the glucoamylase of claim 45.

64. The process of claim 63, wherein steps 2) and 3) are performed simultaneously.


SEQUENCE LISTING (142 sequences)

SEQ ID NO: 1 - DNA, 1633 bp, Saksenaea vasiformis
  source 1..1633: mol_type = other DNA
atgccatcctggaaaacccttttcttgctccttgggccgattgcgactgcagcagctgctccagtggacaagcagtctcttccaaccggaaattctaccattagctcctgggtatccaaacaagaagatattagcttttcggaaatgcttcgcaatgtaaaccctgagggaactgcaaaaggatttgtagctgcatctttatccaccgccggcccagattacttctacacatggacacgcgacgctgctttggtttctcgggtgattgcttacaagtacaacaccaccaacgctggcgacagtaagatccacggggtccttgatgattatgtcaacttccaaatcaacacgcaatcggagagtaccccttgcaattgccttggcgaaccaaaatttaatcccgatggatcaagctttacgggcccctggggaaggtaatactgacctcattgtgcatgggaaagatactaaataattatttgttgaaactcatcagaccacaaaacgatggtccagctgagcgagcttcaagcttcatgttgattgccgatagcttcctgagccaaacaaaaaatgcgtcgtacttcacgaatacattaaagccaggtacgtctaacctctccggaaatacgttttcccctttaatcaccgtctttattagctatctataaggatctcgattacgtcgtcgatacctggagcaatccatgcttcgatctgtggtaagcacacacactttaaaaagttggcgaataaggcctctaattatattatagggaggaggtcaatggtatccatttctataccttgatggtgatgcgacgtagcctcttggatggtgcaaactttgccacgcgtaatggtgacaactccaaggccagtacctatagtggcgtcgctgccaaaatccaagcaagacttaattccttctgggacgctggcaagaattatattactgtcacacaagactataagaacggtgtcgaaaagccatctggattggatgtctctacattgattgctgctaatgtagcaggcatgggcgatggtaacaataacgtagtaaagtctccaaccttatactgacatactattttgcttaggcttctacactcccggatctgaaagaattctcgcaactgctctagccttcgagaagtccatggcaagtctataccctcttaacaacaatttgccttctcaccttggtaatgccatcggtcgctaccctgaagacacttacaatggcaacggaaattcccagggcaacccgtggttcctggctactactgcctttacagaactttactatcgtgccattctcgaatggaagaacacaggtgtgacagtgacgccaatcagcaaagacttctttgtcagatttgattccagtgctgcacccggcaagaaatacaaccccggctctcaagaatttgccacgttgacccagagcatcgccgcagctgccgaccgttttatgtcgacagtacaatatcaccagaatccaaacggatctctttctgaggagtttgaccgctcctcaggatacatgaccggtgctcgcgatctcacctggtcccacgctgcttttatcacagctgctcaagcaagagctggctcaccatccttctaa

SEQ ID NO: 2 - DNA, 2498 bp, Backusella circina
  source 1..2498: mol_type = other DNA
atgaaactttttggtactctaaaggcatcttttgtcttccttattggatgcatagtgtttgctatggcagacactgtccctactacccaggttaaactccagtcctacacttattctggaggtgtattgtctggtacaatctatgtaagagaatgattgcccaagtctatttcgtttttttctcttccaaactcatatattattttgataggttcaaaatatagactacactaaggttgtcactgttatctattctgatggttctggtaactggaataacaacggcaatactatctccgcttcctactcttcctccatttctggaacaaactatgaatactggacattctcagcatcagttagtagcatccagcaattttacatctcagtaagtgtaaaacacaaaaaaagtaggaaatttcagttatttatgcattaatcgattctttcttttttttttagtatcttgtagatggtactacctattatgacaacaatggtggctatggcgccaactatgctggtaagctcaaattcaaattttattttcatttacttaaagatgaataaatagtcactgcaaccaccacaactgcttcatctactactaaaaccacttctggtacaacaaagacatctactacgtcaacagccactgctacttcaacaggcacatcctcttttccttctggtaattctaccatcagtacttggagccccagtcaacacagtatcagtctttacgctatgcttcgtaatatcaatcctccaggaagtgctgctggattcatttctgcctccttgtctacctctggacctgattattattattcctggacaagagtaaatagttaaaatttaaaatacatcatgctgtttgagtgctcattttttgatatttaggattctgcacttgttgctcatgtcattgtcaacgaatataatactacatatcaaggaaacagcacccttctcggtattttgaaggattatgttacctattctcttaacgctcaaactacctctactgtttgtaactgtcttggtgagccaaaattcaaccctgatggttcgagcttcactggtgcctggggaaggtaaacaagattcatgaagagagacgaaaagatattaaccgtttatttagacctcaaaatgatggtccagctgagcgtgctgtcagttttatttattttgcagatagttatctcactcaaactagcgactcatcttatgttactggcacacttgcacctggtaagagtcaattttttttatgatttttgcaaatttcaggaatcttattttgtttcataagctatttataaggatttggattatgttgttagcgtatggagtaacggttgtttcgatctttggtaaattgtatttacttttttccttaattgtaatactaatatatgtttgaattttttttttagggaggaagtaaatggtatccatttttacacactgatgtttatgcgcagaggtctccttgatggagccaactttgcctcacgaaacggtgactctactcgtgcttccacttatacctctaccgctgcttctattaagaccaagattgatggcttctgggtatcgagtggaaactatgttcaggtcagtcaaagtgttacaagcggtgtcagcaaagctggatatgacgcttctaccttaattgctgcaaatcaagccagtcggggtgatggtaagttgtgtctttttttttttaccaaaaaataaaaaaaagaatctcaattaaacaaaattgatgtttaggtttctatacacctggatccgataaggtaacgcaatagataaagttaaaacctctatatgactaacataaagattacagatgctggctacagctgttgctattgaatcccaattctcaagcttgtactcaatcaacaccaacaaggcttcttatctaggaaatgctattggcaggtgagttcctttaacactacattttcttctaaaattctgataatatcatattaataccttattaaaagatatcccgaagatacctataacggcaatggtaactctcaaggcaacccttggttcttgtaagtaaatcattctcataaattatataaattgcatatctttaattttatttttataaaaggtgcactaatgcatttggagagcttttctatcgcgccatcagtgaatggaacgctgctggtagcgtcaccgtaaacagcgtcaatcttgccttcttcaagaaatacgattcttctacctcctctggtacaacgtatacagttggaacaagtgcttacaataatctcgttcagaatgttgctcttgctgctgatgcctacttctccaccgtcaaatatcatgctcttactaatggttccatgtctgaacaatacgatcgtagctctggtatggctacaggtgcaagagatcttacatggtcacacgccgctatgattactgccttgaaggcaaaatctggaacaccagtttactaa

SEQ ID NO: 3 - DNA, 2550 bp, Backusella circina
  source 1..2550: mol_type = other DNA
atgaagcctttcggaccaatcaagaccactctttttattattattagccacttctctttgaattctttagctgatacagttcctactacccaggtagaggtcaaaacttatacttactcgggtggagagctgtctggtacaatctacgtaagtttctcgctctaactttggctttcttctgtatatcaaaatcttatttaaatacacttccttcatatgtttaggtcgagaacattgattatactaaggttgttacggtcatttatgctgatggttcagatgactggaataacaatggcaataccattgctgccgccttctctgaatcaatctctgatactaattacgaatactggactttctccagttctgttaacagcattaaggagttctatgttaaggtgaatagtttattcaatacttttaattatgattttaccatataaagattatacatatgcttatcattcacttattaaatagtatgatgttgatggaaacacctactatgacaataatggaaatgccaactaccaaggtatttttttttatcaaacttaatatttgtcaatgttaacgctaacacatgatcatatagtctctgcttcttccactaccacaactactgctacaaagactacaaccactgcaacaaaaacgactgctactgccacatctactgccactggttcaacttcattcccttctggtaactccactatctcttcctggattaagggtcaagaagataccagtcgatctgttatgcttggcaacattaacccacctggaagtgccactggttttatctctgcttctttgtcaactagtggtcctgattattattatcattggacacgcgtaagtcaattccaatttgaacattatagaagttacctctctaatattcgaaaactaacttttctcatttatctattttcatcaggatgctgctcttgttgctcacgttattgtcaatgattataatactactctttctggagatagctctactcttcaagttattaaggattatgttactttctctgttaacagccagtctgaatctaccgtatgcaactgccttggtgagcctaaattcaatccggatggatcaagctacactggtgcatggggcaggtaagatattatagcatttaataaaatcatgcgcatgttatattcattattaattttgtataacattaatcagacctcaaaacgatggtcctgctgaacgtgccacaacattcattttctttgctgacacttatctcgcacagggtggtgactctagctatgtgactggtacacttgccccaggtatatatagccttttaaaaaacaaattactttaaaaagtgaatagtattactatgctaactacatttatatattctttactattttgatttaaatagctatctatgctgatctggattatgttgtaaataactggagcactggatgttacgatttgtggtaaagcatctaatatgaagttaatttttgtgaagtatactcaccaaaagctgatatagggaggaagttaatggtattcatttctacactttgatggttatgcgcaggggtcttattgacggtgccagctttgcctctcgtaatggtgactctactcgttcaagcagctatacttccactgctaaatcaattgctaccaagattgatagtttctggtcagcctccaacaactatgttgctgtcagtcaaagtgtaacttctggtgtcagtaaggctggatatgatgcttctaccattattgctgctaaccaagctagtcttggtgatggtttctatactcctggttcagataaggtaggctaatcttaatgcaatattataaacgtagaaaaagaaacatgtatattaatgccaattgagtagatgcttgccacctccgtcgctgttgagaatgctttctcttctctttacaccgttaatactggaaaggcttcttacttaggaaatgctattggcaggtgagtgcatttgacacttcattttcttcgtataatttctcactaaaaaaactactaccatattaaaagataccccgaagatacctataatggtaatggcaactctcaaggcaacccttggtatttgtaagtaaaactcttttatgctcgataattatcccttcctaacacatcatttttatttttaaaaaaaaggtgtactaacgctcttggtgagttatactaccgtgcaattaaggaatggaatgccgctggatccgttacggtcaacagtgtcaatctcggcttcttccaaaagattgattcctcaatttcatctggcaccacctttaccttaggcacttctgactacagcaccctcgttgacaatgttgcacttgctgctgataagttcttcgccgttgtccagtatcatgaacgcagtaatggatctatgcctgagcaatttggccgtgaggacggtcttcctactggtgctagagatcttacatggtctcatgctgccatgatttctgctgcccgtgctaaggctggtactcctgtgtattag

SEQ ID NO: 4 - DNA, 2256 bp, Benjaminiella poitrasii
  source 1..2256: mol_type = other DNA
atgaagttttctcatctcttcaggaagccaacgcttcttattgtgacctttttgaccgctacggtttttgcagaaactgttcctacaacttctcaagtcaagcttaagagttatacatatgatggttccaccttatccggccagatttatgtaagttatattaattattcactttatattttttttttcccctgacctgtcaaaaatatactaatttttatttcaaattttaccagattcaaaatattgcttatacaaaggtggttactgtaatctactctgatgcgtctagcaactggaacaacaatggaaacacaattgctgcttcatatactaatggtatctcaggaaccaattatgaatactggaatttcactggtcctgtcagcagcatcaatgcattttacatcaaagtaaattactgaataataatgttcacaagcaattataaattctcactttaattgattatatagtatgatgttagcggaaatacatattatgataacaacaataatgcaaactatcaaggtaaaatagtcaagagaaaaaaaaaaagacgcattaaccgaactcaatttattaatgcattcttataatagttacaaagacatcaactacaacgactacttccactgctactgctactactactaccaaaacttccacaccgacttctactggtgtcccctccaatttccccaccggtaacagtactattagcacatggttgaaatcacaaattggcatcagtcgctatgctatgctccgtaacatcaatcctgctggttccgctgttggttttattgctgcctctttatcgactgctaaccctgactactattatgcctggactcgtgattctgctttgacctcatatgttattgccaacgattataactctactttggctggtaactcaacaatccttcaaatcatgaaagattacgttactttctctgttaagagtcaatcggtttctactgcctgtaactgccttggtgaacccaaattcaatcctgatggatctagttacactggcgcttggggaaggtatgtagataataccataccttttttttttttttcataatgatatacatgtaaaaagtttaactaacatgatcaatatgtaaatacaaagaccccaaaatgatggtcctgctgagcgtgctactacatttatattatttgccgatagttatcttaagcaaacaggcgatgctagctatgttactggaacccttaagccaggtaaagttttttcctttattacggcatgtatattatgttaacatatacacgtaatttgaatagctattttcaaggatttggactatgttgtcaacacatggactaatggatgctttgatttatgggaagaggttaacggtgttcacttctataccttgatggtaatgcgtaagggtttgatcagaggagctaactttgccacccgcaacggcgactctacacgtgcttctacttacaccaacacagctgcatccatcaaaacaaagatggattcattctggtcatctggcaacaactacattgctgttagtcaaagtgttactggtggtgtcagtaaggctggttacgatgttgctaacattattgctgctaatgttggcagtctccaagatggcgtatatactcctggttctgatagagtatgtatcaacagtaactttatagtatcaacagagacaatcatattaaccattgcacccccccttttttttctagattcttgctactgctgttgctgttgaagctaaatttgcttctttatatggtgttaactcaaacttaccaggctaccttggtaactccatcggtcgttatcccgaagacacatataacggtaacggaaactcccaaggtaacccatggttcttggccacaaatgcttacgcagaactttactaccgtgccatcagagaatggtatgacaatggtggtgttacagtcaacagtgttaacttgccgttcttcaagaaattcgatagttccgctgcttctggtacaacctacactgtaggtacgaccgccttcaacacgatggtttccaatgttgcagctgcagctgataagtttttctcgactgtcaaattccatgcttacaccaatggctccatgagcgaacaattcggtcgtaatgatggtttatgtacgggggctcgtgatcttacatggtctcacgcttctttgatttctgctgctcttgcaaaggctggtactccttcagtctaa

SEQ ID NO: 5 - DNA, 1968 bp, Choanephora cucurbitarum
  source 1..1968: mol_type = other DNA
atggtttcttttaacttccttaaaaagccagtattcattgtgattacttgcttgacagtctctgtctgtgctcaatctgtgcctaccagtgaccctgtcaaggtcaagactttcagctacgatggtaactctttctctggtcaaatttatgtaagtgtcaatacttatttggaaggtacgggggtttaattgtttaattggatttcgcagatcaaaaatattgcttatgaaaagaccgttactgtcatctactctgatggtactggtaattggaacaacaacaacaacaagatcgctgctgctttctctgaagccatctctggttccaactatgaatactggactttctctgcctctgtccctagcattaaacaattttatgtcaaggtaaagaaacaatagttttaaacataccaatctaatgtgccttcatagtatgatgtttctggtaaaacttattacgacaacaatggtagcaaggactataatggtaaaatcaaattttcacatgaattaagagtatgttacactaaaccttttttttttttaatagttgttacctctggtcctaccaccacttccagtggtccctcaaagacaaccaccaatggtcccgctcctacctccaccaacactaacttcccctccggtaacccttccatcacttcatggatcgataagcaaattgacatcagtcgctctgccatgcttaagaacatcaaccctgccggtaccgtcaagggtttcattgctgcctctctctctacctcaaaccccgattacttctacgcctggacccgtgacgctgccttagttgcccatgttgttgctaacgactacaaccgtaccaagagcggtgatgccacttacctcggcctcctcaaggattacgttaccttctctatcaacagtcaaaacacgcctactgcctgtaactgtctcggtgaacccaagttcaacaaggatggttctggttacaacggtccctggggtcgtcctcaaaacgatggtcctgctgaacgtgctgacactttcgtcttgattgccgacagtatcttgacccaaaccaaggacgtcagctatgtcactggtaccttgcgccctgctatctacactgatcttgactatgtcgtcagaacctggtccaacggctgtttcgatctctgggaagaagtcaacggtgttcacttctacaccttgatggtcatgcgtcgtgccttgcttgtcggtgccaactttgcctctcgtaacggtgacagtgctcgtgccagtaactacaacaacgctgctaactccatcaagtccaagatcgacagcttctggtcttccaacaacaactatgttgccgtcagccaaagtgtcactggtggtgtcagcaaggccggttacgatgtctctaccttgattgctgccaatgtcggtagtctctctgacggtttctacacccctggttctgaaagaatgcttgctactgccgttgctatcgaagacaagtttgccaacctctatggtatcaaccgtaacttggacagcagccttggtaacgctatcggtcgttaccctgaagacacctacaacggtaacggtaactctcaaggtaacccctggttcattgctaccaacgcctatgctgaactctactaccgtgccatcaaggaatggaacaacaacggtggtgtgactgtcaccaacgtcaacctcaacttcttcaagaagttcgatggtagtgcctctgtcggtaccaagtacactgctggttctgctgcctacaacaccttgactcaaaacattgctcttgctgctgataagttcttcaacaccgtcaaggtccatgctgccaccaacggttccatgtctgaacaataccaccgtgacaccggttccatgacaggtgctcgtgacttgacttggtctcatgccagtttgattactgctgcccttgccaaggctggtactcccgtagcttaa

SEQ ID NO: 6 - DNA, 2204 bp, Rhizopus stolonifer
  variation 171..190: note = Any nucleotide
  source 1..2204: mol_type = other DNA
atgaaactgtttggtttgccattgaagccttcgctcttttttgtggtctcttatctttctttcctcgcctctgctgctagtattcctagtagttctgaagttcagcttgacacatacgcgtatgatggtgctaaattttcaggaaaaatttatgtgagttttttttttttnnnnnnnnnnnnnnnnnnnnttttttttttttttttttttttcttctaaaggaaggtatctgatctcaaataaatttaggtcaaaaacattgcttatgaaaagactgtcactgtggtatacgctgatggttctgataactggaataacaatggaaatataatttctgctactttctctaccccaatctctggatctaactacgaatactggaccttttcttcttctattagtggtattaaagagttttacattaaggtatagttcattttctttatatgtataatttaccaaccttactttttacagtatgtggttagtggtaaaacctattacgataacaacggcactaaaaactatcaagtttctaccacttcctctactaccactgcttccaccacaaccgctactcgtacaactacagctggaactacttcgacctctactacggctgcacctacaagcacttcatccggttctttcccttcaggtaactcgacggtatcctcttggattaaacgtcaagaaaaaataagtcgcttcgctatgcttcgtaatatcaaccctcccggaagcgctgctggttttatcgctgcttctcttagtacctctggccctgactactactactcatggactcgcgactctgccttaacttccaacttgattgcctatgaatataacaccactttgtctggaaacaccactatccttaacatccttaaggattacgtcaagttctctatcagctctcaaacaacttccactgtatgtaattgtcttggtgaacccaagttcaaccatgatggctcaagttacacaggcgcttggggaaggtagttacacatatttttttctggatgagaacacctgtttattaatattaacttagacctcaaaatgacggacctgctgaacgtgcaaatacttttatcttgttcgctgacagttaccttgatcaaacaaaggatgcatcttatgtcactggcactcttaaacctggtaatttttttgaattttatttattgtatagatatgtgtacttatagtttcttaaagccatttttaaggatttggactacgttgtcaatgtatggtctaacggttgttacgatttatggtaaaaaaaaatatgatgtttctttataggatcttattgacagtagaaaatagggaagaagtcaacggagttcatttttacacattgatggttatgcgcaagggcttacttcttggtgctgactttgccaagcgtaacggtgactctacacgtgcatctacttacaccaatactgcttctactattgctacaaagatttctagcttctgggtatcctctagcaactggattcaagtcagtcaaagtgttactgctggcgttagcaaaaagggattggacgtatcaaccttattggctgctaatcttggaagtgttgaagacggtttctttaccccaggttctgaaaaggtaattgtagtttatcatggataagagtatggttttcgagctaaaatgcacatttaaatagatccttgctaccgctgttgctattgaagatgcttttgcttcattgtatcctatcaactccaacttaccttcttaccttggtaattccatcggaagatatcctgaagacacctacaacggcaatggaaattctcaaggcaacccttggttcttggccgtcaatggctttgctgagctttactaccgtgctatcaaggaatgggtcaacaatggtagtgttactgtgagcaacatcagcttgtctttttttaagaaatttgactcatctgctactgctggtaagacctatactgccggtactgcagactttaacaatctcgcacaaaacattgcacttggcgccgatcgtttcttatcaactgtccagactcatgcctttaacaacggatctcttgctgaagaatatgatcgtacaactggagtatctacaggtgctagagacttgacttggtctcatgcctctttgattactgctgcttatgcaaaagctggttcacctgctgcttaa

SEQ ID NO: 7 - DNA, 2015 bp, Mucor circinelloides
  source 1..2015: mol_type = other DNA
atgaagttctctaaccttctcaagaagcctctccttttgatcgctggtattttggccgctactgttgtcgctgagactgtacctactaccgctgcagtcaaagtcaagtccttcacatatgatggatccaccttcgctggtcaaatctacgtacgtactaaaatttcgatttattgaattcaatgctaaacccgcattcttcattctatacaggtcaagaacattgcttacactaaaactgttaccgtgatttactctgacgcttctaataattggaataacaatggtaacacaatcgctgcttcttacagcgaaggcatttcaggcaccaattacgaatactggactttcagtgcccctgtcagtggcatcaagcaattctacgtcaaggtaagtctgggatcactcagtggtcggttgcaagtattgagcctataatctattagtatgttgtaagcggtaccacctactatgacaacaacaacagcggaaactaccaaggtatttacactttcacatgttgcattagtattgtcatactgaaaaaaaaattccgtggcattcctttagttaccactactaccactactactgctcctacctctactacttctggcggttcttctacaaccaccggcggctctactaccactgctactagcgtcccaactggtgttccttctggtttccctactggtaactcttctattagctcttggatcgacggccaaacatctgtcagtcgttatgccatgctccgcaacatcaaccctgctggcgcagtttctggtttcattgctgcttccatgtctacctctggacctgattacttttatgcctggactcgtgattctgccctgacctctcatgttgttgcttatgattacaacactactttggctggtaactctaccattctcggcctcttgaagaactatgttactttctctatcaacagtcaaactacttccactgtttgtaactgtctcggtgagcccaagttcaacaaggatggtagcagttacactggtgcctggggtagacctcaaaatgatggccctgctagtcgtgccgatactttcattttaattgctgacagtatcctcaagcaaaccggtgatgctacttatgttactggcactcttgctcctgccatctacaaggatttggattatgttgtctcaacttggtccaacggttgtttcgatttatgggaagaagtcaatggcgttcacttttacactttgatggtgatgcgtagaggtttgatcaagggtgctaactttgcctctcgcaacggtgacaacactcgtgccaacacttacaccaacactgccgcctccatcaagaccaagattgatagcttctggaactccaatggcaactatgtcactgtcagccaaagtgtcactggcggtgtcagcaaggctggttatgatgcctctgtgttgattgctgccaacttgggtagtgttcaagatggtttctacactcctggttctgacaagatgcttgctactgctgttgccattgagagcaagtttgcttccctctattctatcaaccaaaacttaaacggttaccttggtaatgctatcggccgctatcctgaagatacttacaatggtaatggtaactctcaaggcaacccttggttcatctgtaccaatgcctttgctgagctctactaccgtgctatcaaggaatggtttaacaatggtggtgttactgtcactagtatcagtttgaacttcttcaagaaatttgattcctctgctgccgttggtaccaaatacactgttggtacctctgctttcaacagcttggtccaaaatgttgctgttgctgctgatgccttcttctccaccgtcaagttccatgctgctactaacggctccatgagcgagcaatatggtcgtagtgatggtttgatgaccggtgctcgtgatttgacttggtctcacgcttcattgatttctgcttcttacgcaaaggctggttcccctgctgcttaa

SEQ ID NO: 8 - DNA, 2398 bp, Dichotomocladium elegans
  source 1..2398: mol_type = other DNA
atgaaacttaacagtgtttggaaagcattcagtattttgcttatggccaccatggcttttgctgccaccgttcctaaaacgcaagtgaagcttgaggcgtacacatactcggattccatattctctggtagaattttcgtaagaatctatatctcctcaaatcgtttgtatgcataaaggctcatatattgtgaataggtgcaaaacatcgactacaccaagattgtaaccgtttactggtctgatgcatcgaataaatgggactccagcaaatattatactgaagctgcttacacccattcgatccctggaactaattacgaatactgggatttctcagccaccattggtccttcaggaatcaagcagttctacatttcggtaaccatttattgaggagaatcggtaaatccacgcacctcttttactcatcgtgttcggatgactttcagtatcaagtccggggtgtcacttattatgataacaacggcggctatggtgtcaattacggtgagcaaaacactgtcagctagtagctcttcatgccctacttacaaatgcgttgtagacgtcatctcgtcacccccgaccacgtccgatgccactaccattcctactactaccactagtgctattccttcttccactaccactattcccgccagtactggggttccttctggaaactccactatcaccgtttgggctgattcgcagcataagatcagctggaaagcaatgctcgccaatattaatccacccggttctgctactggttttattgccgcctcgctttcgacttctggtccagactactactatgcctggactcgtgatgctgcgatggttgcgcacgttattgtcaacgcttacaacacaaccaaagcaggtgatgcaactactcttggcgttctgaaggactttgttaccttccagattaaggctatggctaccagcaccgtctgcaactgtctcggtgaacctaaattcaaccctgtaagttaccagatagtacagcagaaagaaaataccgtctttaccatcggtcgtaggatggctccagctatactggtccttgggggagaccacaaaatgatggacccgctgagcgtgctactactttcattctatttgcggacagctatctttcccagaccggagacgccgcttatgtcactaccttgaaaagaggtaagcatcctggaagcttcaagtgtgcagtgattaacctgtaaattatgcttcttccacttcattagctatctttaccgatcttgactatgtggtgaccacctggcaggataattgttttgatctctgggaagaagtgaacggacttcacatgtatacgtaagttgttattcctctttttctcgtatgcacacgcatacccgcgcccggagtattagcagaaaaaagcttaagctactaatggaactcttatttcagtttggcggtaatgcgccgaagtctggttgatggagcaagctttgccgcccgcaatggtgacaatacacgcgccagtacctataccaatactgccaaatccatcgagaccaagcttgcaagcttttacaactcgagtagcaactatgtcgttgtcaccaagaacttcgcaggtggtgtaaacaaggctggtttagatacctccacattgatcgccgcgaacactgccggtttgggtgatggtaagcgggaagcatgatgattaatctgacccttagagagggagaaaatagatagctctcttttcaatatgtgattctgactgccagccaacctctccccaaggtttcttcacccccggttcaccggagattctagctaccgccgccgcaatcgagaagtcattcgctgatctatacggcatcaacaaacaaattcccaactggttaggcactgcaattggccgatacccggaagacacttacaatggcaacggcaactcacagggtaacccggtaagtatccctccaacctttgtctctctcttcgttgtgggacctataatgcgatgcttatctatttaacctagtggttcatctgtactgctaccttcgccgaactctattatcgcgccattctggaatggcaacacgctggctccgtcacagtgaacaacgtgaacttgccattcttcaaaaaattcgatagctccacttcctctggcaccacctacactgtcggcactagtagctttgattcacttatctcaaaagtagcctatgctgcggataacttcttttctacgatcaaatatcacgccgctacgaatggttccatgagcgagcagttcaatcgtgataccggttttatgactggcgctagagacttgacctggtctcacgcggcattcatcaccgctgctaaagccaaggctggcactcctgtatactag

SEQ ID NO: 9 - DNA, 2311 bp, Fennellomyces sp.
  source 1..2311: mol_type = other DNA
atgagaccaagtcttgtttggaaaagcctgcttatggttgtgcttaccgcagccatgggcacatatgctcaaactgttccatctgtgcctatcaagttggaaagttacacttactcggataacgtattcgccggtcgaatctttgtaagtagttcgataatttctgtttgttaaagagaaaaaaaaactcacccctttttttcgtcacgtagattcgaaacatcgcttatacaaaagtggtcaaggttttctggtctgatgaatctggcaattggaacggtaacggaaactatgttgatgcctcgtattcggggtctatttccggtaccaactatgaatactgggaattttcaacaaccatcggatccgccggtattagccaaagctatttgagagtaaagagtttgtcctttttaagtgtttcgcaagtggggaagagagactaaacagaatattttctttttagtatgatgtttcaggttctacgtactatgataacaatggaaatgcaaactatggtaacgtattaactagaagcggtcaaatttgaaatcactcaaagccttcttttacgtgtttcagatatcgaggaagactccacacctacgcccaccaccaccactaccagtactacgacaataaccactaccactaccagtggaacttcaactcctacttctgtccccactgaagttcctgctggcaataccactgtcactgactgggtgaatgatcaattggaaattagttggccatcgcttttaaagaatgtcaatccttccggcgcagtaaccggtttcatcgctgcttcgctttccacaaatgatcccgattatttctactgttggacgcgtgatgccgctttggtcgcacgagtgatggtttacatgtacaacacaaccgaagctggtgacaccagtttgagaagcaagctgcaagattatgttacattccagatcaactccatgaagactgcaacagtatgcaactgcttgggtgaacccaaattcaataaggttagtagttttttttttaaatgatgatacacttttgacaaaaaagaattaggatggttcgggttactcgggagcatggggtcgcccacaaaatgatggcccggctgatcgtgctatcacgttgatcctgtttgcggacagtttcattgctcaaggcggtgatgtctcgtacattaccaacacattgaagccaggtaagtaatcgggtatcaccgtaatccggtctccaattactaatgtatgccacagctatctacaccaatctcgactatgttgttaacacctggagcaatgtttgctttgatctatgggaagaagtcaatggtgtccatatctatacgtaggttttttcgggggagagggaaacgttaatgtgaaactttttttttgaagtactcattgaaaatgcttatttagactttcggtcatgcgtaagggtttgcttgaaggtgccgactttgcttctcgaaacggcgattccacgcgtgccaacacttatagaagcaccgcttcttccatcaagacccgtcttgaatctttctggtcatcgagcaataattacatcactgttactcaatcatactctggtggtgtgaacaaggccggtttggatgtatccaccttacttgcagccaacggagcaagcatgaatgatggttagtttttgcttaaaaaaattgactttgatatttaccaaaagaatactgcgtaggcttctatactcctggctctgacaagatgttggcaaccgctgttgctattgaaaattcgtttgccggtatttacgctgttaaccagaatcgacctgattggaaaggtactgctattggtcgatatcccgaagatacttatgatggccatggcaattcccaaggcaaccctgtaagtcatatattccttatcacaagaatgaattctaacttagaaaatagtggttcattgccacagctgcgtatgctgaaatgtactaccgtgccatcttggaatggcaacagcaaccgtctatcactgtgaactcgatcaacctgagctttttcaaaaagtttgactcgtccgccgctgttggtactgtgtacaagcccggtacccaagctttcaacaacatggtttccaatgttgcgttcgctgctgatgagttcttttctactatgaatttccattccgccaccaacggatccatgagcgaacaatacaaccgcaatactggtattatgcaaggtgctcgtgatcttacttggtcccatgctgccttcatcaccgctgccaaagctaagcttggcacgccagtattctag

SEQ ID NO: 10 - DNA, 1985 bp, Gilbertella persicaria
  source 1..1985: mol_type = other DNA
atggttttctccaagctttttaagaagcctatccttctcattgtcacttacttgactgttactgttgtagctgaaactgttcctacttcagctcaagttcaagttaagtccttcaactatgatggctctactctttcaggtcaaatttatgtaagtaattgagaggaagcgaatagtcattctgaatttgctttaggttcaaaacattgcttacgaaaagactgttactgtcgtttactctgatggctctgataactggaataacaatggtaacaccattgctgccagctattcttcttctatttcaggtagcaattacgaatactggactttctcttcttctgttcctagcatcaagcaattctatatcaaggtataaaaaaaaaaggaaacatgtatgacaaattagctttactaacatcatcatctgtattcagtatgtcgttgctggcaagacttactatgataacaatggtacaaagaactatcaaggtatgtaattcacatgttgcatgaaatatcagttacactaacacaaaggctttaattgtatcaatttagtgagtgcaagcacccctactaccactactaccactacttcttctggagccaccaagactactactaccattggccctacttctacaagtactgtcttcccctctggcaactctaccatttcttcttggctcaagggtcaaattgaaaccagtcgttttgccatgctccgtaacatcaaccctgctggtactgtcaagggtttcattgctgcttctttgtctactgctaaccctgactatttctatgcctggacccgtgatgctgctttagtcggccatgttattgccaatgactacaaccgtaccttagctggtaacagcacttaccttggtcttttgaaggactatgttactttctctgtcaactctcaatctacttctactatctgtaactgtctcggtgaacccaagtttaacaaggatggttctggctacagcggtgcatggggccgtcctcaaaatgatggccctgctgaacgtgctgatacattcatcttgattgctgacagtatcttgaaacaaactggtgatgctacctatgtaactggtaccttgcgtcctgctatctacaaggatttggactacgttgtcaatgtttggtctaacggttgtttcgatttgtgggaagaagttaatggtgttcacttttacactttgatggtcatgcgtcgttctttgattttgggtgccaactttgcttctcgtaacggtgattctactcgtgccagtacttacaccaacactgccaactctatcaagaccaagattgacaccttctggtcttcttccaacaactatgttgctgtcagtcaaagcgtcactggtggtgtcaacaaggctggttatgatgttgctaacttgattgctgctaatgttggtagtctcgatgacggtttctacactcctggttccgaaagaattctcgctactgctgttgctgttgaaagtaaatttgcttctatttacggtgttaaccaaaacttgcccagctggctcggtaactctatcggtagataccctgaagacacttacaatggtaacggtaactctcaaggtaacccttggttcattgccactaatacttatgctgagctctactaccgtgctatcaaggaatggaccaacaacggtggtgtcaccgtcactaatgttaacttcaacttcttcaagaagtttgatagcagtgcttctgttggtaccaagtacactgttggtacctctgcctttaacaccttgactcaaaatgttgctcttgctgctgacaatttcttctctactgttaaggttcatgctgccaccaatggttctatgtctgaacaattcggtcgtgatagcggtgtcatgactggtgctcgcgatttgacttggtctcacgccagtttgattactgctgctcttgccaagacaggtgctcctgtagcttaa

SEQ ID NO: 11 - DNA, 2018 bp, Mucor circinelloides
  source 1..2018: mol_type = other DNA
atgaagttctcaaatcttctcaagaagcctctccttctgatcgctggtatcttggctgtcactgtagtcgctgaaactgtccctactactgaagcagtcaaagtcaagtcctttacctatgatggatctaccttggctggtcaaatctatgtaagtgctagaatcagtaccaatcccttcaataaacacaatgctaaatacatgctctcaacgaatagatcaagaacattgcttacaccaagacggttactgtaatctactctgatgcctctgataattggaacaacaatggtaacacgatcgctgcttcttacagtgcagccatcgcaggcaccaactatgaatactggactttcagtgctcctgtaagcggcatcaagcaattctacgtcaaggtaagtttgggattcctgtatgctgggtgctcagatattaagcctctgatccgtattagtatgttgtcagcggtactacctactatgacaacaacaacagcggaaactaccaaggtatattgattttcacatgctggcatttgcactgtcatactaaacgagctgaatcctctagtttcagtcaccaccactaccactactgctcctaccactaccacttctggcggttcatccactaccactggcggctccactactactgctactagcgtccccactggtgttccttctggtttccctacaggtaactctaccattagctcttggatcgatggtcaaacctctgtcagccgttatgccatgctccgcaacatcaaccctgctggtgccgtcactggtttcattgctgcctctatgtctacttctggtcctgactacttttatgcctggacgcgtgattctgccttgacctctcatgtcgttgcctacgactacaataccactttggccggtaactctaccattctcggtctcttgaagaactatgttaccttctctctcaacagccaaactacctccactgtctgtaactgtctcggtgagcccaagttcaacaaagatggcagcggttacagcggcgcctggggtagacctcaaaatgacggacctgccagccgtgccgacactttcatcttgattgccgacagtatcctcaagcaaaccggcgatgctacctatgttactggtaccctcgctcctgccatctacaaggatttggattacgttgtctctacttggtccaacggctgtttcgatttgtgggaggaggtcaacggtgttcacttttacactttgatggtgatgcgcagaggtttggttaagggtgctaactttgcttctcgcaatggtgacagcacacgtgccaccacttacaccaacacagctgcttccatcaagaccaagattgacagcttctggaactccaacggtcaatatgtctctgtcagccaaagcgtcactggtggcgtcagcaaggctggttatgatgcctctgtcttgattgcttccaacttgggtagtcttcaagatggtttctatactcctggctctgacaagatgcttgctactgctgttgccattgagagcaagtttgcttctctctactctatcaaccaaaacttgaacggttaccttggtaatgctatcggtcgctaccctgaagacacctacaacggcaacggtaactctcaaggtaacccttggttcatctgtaccaacgcctttgccgagctctactaccgcgctatcaaggaatggttcaacaatggcggtgtcactgtcactagcatcagtttgaacttcttcaagaagtttgattcctctgctgctgttggcaccaagtacactgtcggcacctcttctttcaacagcttggttcaaaatgttgctgtcgctgctgatgccttcttctctactatcaagttccatgctgccaccaacggttccatgagcgagcaatacggccgcactgatggcttgatgactggtgctcgtgatttgacttggtctcacgcttcattgatctctgcttcttatgcaaaggctggttctcctgctgcctaa

SEQ ID NO: 12 - DNA, 2946 bp, Circinella umbellata
  source 1..2946: mol_type = other DNA
atgatgccgaatcatatttggaagtgtctgtttctagccatgaccacagttttcatgactgtactggtacaaggtgctcctacttcagaaattgaattggactcttatacctacaccggtggagtattttctggtcggttatatgtatgtggtatctatttgtacataattaaaagaaaaattcaagagaatacagttttgttaatgttggtaattcccaatatatacaggttaaaaatattgcgtacaccaaggaagtgaatgtctattggtctgatgcttctgaagactgggccaataatggtaattatgtggcagctacgtattctgaagccatttcgggtacaaattacgaatattgggaattctctgccaaaattggatcttctggtattagtgaaagttacataaaggtaaattactataaattgtttgaaactccaaaatacaaaataaaaaatccggcatagttattaattattctttatttttttcaaataaattcagtacactgtgtctggctccacttattatgataataatgggggtaaaaattatggtatggtcaaaacattttacacagtaataacgcctttcttgcatcatcgaacttttaaagtatatgcttatttttttttcgtcattttacaatagctattactgagacaactacacccacaataactgcaaccccgacatcaacaacggcgacaacgaccatttcttcaacaactactacggctacttccattcctacctctgttccttccactgttcctgagggaaatgttactgtcacagaatgggtcaataagcaactaaaaatcagctggtccgatcttctgcaaaatgtaggcgtataataactaaaaataccttatattatattctatgcatactatctgtaaaaaaaaccttaaaaattattgaaaacctgtttctcattatttatattacttctatcttatatataggttaacccttcaggtacagttactggttttattgctgcaagtttaagtaccagcaaccctgattacttttactgttggacaagagatgctgccatggttgcacgtgttatgacttatatgtataatactactgaagctggcgactcaagtcttgaaagtgcattaaaagattatattacgttccaaatcaattcaatgaagactgccactgcatgtaactgcttaggtgagcccaagtttaatactgtaagttacgtattagagtaagaaataaaaccccctcgattgatcactagacatgttatctgtatcacgtatcattacaatgctaattttttatttattgacaaatgtggtactgtaggatggaagtggttatagtggcccttggggtaggcctcaaaatgatgggcctggtaagtttaaactattttttctattttattttactttataagatttttaaaaatatgttgatcttatgatgatcttatgccttaacaaaaatgttttttcatatttatacatatagctgaacgtgccactacaatgattttgtttgctgatagttatctcgctcaagggggtgatacttcctacgttacaaatacattaaaacctggtaaatcattataataatattttgtatactataacaaacacagatagatgtaaaaaaaggctttctgtttctcatgtatattaacaaaaacaatttaccttttttttttgttattaaaacgattatatagctatctataccaatcttgactatgttgttggtacctggtcaaataattgtttcgatttatgggaagaagttaatggtgttcatatttgtaagcaatatcatgatatttttctaaaaagaacaatacacacacatacacacacacacacacacacacacacacacacacatatatatatatatatatatatattaactgctatcaacatttttacttcataaaaactaaaataacaataataataataatagttactctggctgttatgcgcaagtcattacttgatggtgctgattttgctgcacgtaatggcgatacatctcgtgtcagtggttatcaaagcacagcttcatctatcaagactaagttagaatcattctggtcgtccagtaacaattatatcactgtaactcaatcctattctggaggtgttcaaaaggccggattagatgtttctactttaattgctgccaaccaaggaagtatgggtgatggtaagtttttatttttattttactataataaagtagtttgtaatataaaatacgtaaaaaagaaaagacggatttttttatatgaaatataactattgctctgtaggattctatactcctggctctgataaaatgctggctactgccgtcgctattgaaaactcatttgcaaatatttatactatcaataaaaacaaaccttcttggcttggtactgctatcgggcgttatcctgaagacacatataatggtaacggcaatggtcaaggcaacccggtaagtttatatataacccatttactaaaagaaggtttcattttatatatgtatctaatatattatatggtcactactattatttagtggtttatcgcaacagcaacctatgccgaattatactaccgagctattttggaatggcaacaacaagagtctgttactgtaaactctgtcaactttgatttctttagcaagtttgattcatcggctaaggttggcactgtttatacaccaggtacagatacctttaacaccatggtttcaaacgtagcttttgccgctgatgaattcctttcaacaatggaacattatgctgctacaaacgggtccatgtctgaacaatttaatcgagagactggctctctcaccggagcccgtgatttgacatggtcccatgccgcatttatcacagctggtaaagctaaattaggtattccatcattctga

SEQ ID NO: 13 - DNA, 2054 bp, Mucor cordense
  source 1..2054: mol_type = other DNA
atgaagttttccaagctctttacgaagccaactctcttcatagttagcatcatcatttctgttgcttcctctgaaactgtccctactactgccaatgtccttgtcaaatcttacacttgggacggtgccactttatctggtcaaatttacgtaagtatattatgtaatggcgtgttatagcaaaaattgacactgcttgtctatagattaaaaatcttgcctatgccaaggttgtctctgtcatttactctgatgctaatgataactggaataacaatggtaacaaagtagctgcctcctactctgctggaattgatggaacaaactacgaatactggaccttttctggcgctgttagcggtatcaagcaattttatgtcaaagtaagtatagatcttctatattcaaattgaatatataaactaactgtataatgtagtatgatgttagcggtactagttactatgataacaatggtaccaagaactaccaaggtaaaaattagataatgaagtaatcgtatacagatccttacatgtcgtcttcatagttaccaagacctctacaaccaccactgctgctactaccactaccactgccactactactactggtggtactggtaccactactacaactaccgctactgccactgctacatctactgatttccctacaggtaacagcactatcaccacttggctcaaatctcaagaagacatcagtcgtggtgctatgctccgcaacatcaaccctcctggtgccgctacaggtttcatcgccgcctcattatccacttccggacctgactactactatgcctggactcgtgattccgccttaacctcccacgtcattgctcacgactacaacactacattagccggtaacaccaccattctcaacatcctcaaggactacgtcactttctctgtcaagagtcaatctgtttccaccgtctgtaactgtctcggtgaacccaagttcaaccctgacggctcttcttacaccggcgcttggggtcgtccacaaaacgacggtcctgctgaacgtgcttccacattcatcctctttggtgacagttacctcaagcaaactggcgatgctacttacgtcacgggcacattggctcctgctatctacaaggatttggactatgtcgtgaatacttggtcaaatggttgctttgatttatgggaagaagtcaatggtgttcacttttacaccttgatgagcatgcgtagaggtttgcttgatggtgctaatttcgctaagcgtaatggagatactacacgtgccactacttacacaaataccgctgcttctattgctacaaagatcgataccttctgggtctcttctggtaactatgttcaagtcagtcaaagtgtgactggtggtgtaagcaaggctggttatgattgtgctaatattctcgctgccaacatggccgccaaccacgatggtttctatactcctggttcaagcaaggtaaatatacatgactcactatctctttatgaaagtatactaaacgtttcatatagattcttgctactgctgttgctattgaatccaagtttgcttccttatacagcattaactctggtttggctagctggctcggtacagctattggtcgttatcctgaagacacttacaacggtaatggtaactctcaaggtaacccttggttcatctgtaccaacgcctttgctgaactctactaccgcgctatcaaggaatggactcaagctggttccgtcactgttgacagcactagtcttaacttctttaagaaattcgatagctctgctgctgcaggcacgaaatataccgttggtacttctgctttcactaatttggttcaaaatattgctaatggtgctgataagtttttgtctacttccaagttccatgccgctaccaatggttccatgtctgaacaatataaccgtgattctggtttgatgacgggtgctcgtgatttgacttggtctcatgcttctttgatctctgcttctagagctaaggctggctctccttctctttaa

SEQ ID NO: 14 - DNA, 2709 bp, Phascolomyces articulosus
  source 1..2709: mol_type = other DNA
atgggtgtattagcagacgtccctacctctgccaatattgaactggattattatacttataaggataaagtattctctggtagaatttatgtatgaattttaaaatagagtaaaatgaaagaaaaaaaagatagaagaatgggaatatataataattaaaggttataatatatattttattaaataggttaaaaacattgcatatgagaaaacagtagctgtctattggtccgatgcttctggtgattggaataataacggaaataatgttgcagcttcgtactctgaatctatttcaggtactgactatgaatattgggatttctcaaccacaattggatctggtggtatcaagcaaagttacttgaaggtacacttgattgttatcatcctgttatttttcaaattataaatatatatactaaaagtacacatactcaatagtatactgtatctggtaatacttactatgataataatggaagcaataactatggtacgtctcaatttaaaaaccatataaacaagtatatttctgtattcgtattcaggaacgtatgaagctgacatataagtttttgattatagatattaccgagaccactgctactactactactaccacaacaacaacaaccactactggctccaccgcaacaacatcaacaacttctggtcctaccagtactaccagtaccacacccggccctacacctaccaatgttcctgatggcaatgtcactgttacagagtgggttaacactcaatttgccatcagctggcctaccctcttgaaaaatgtatctatcaaaaaaatataatccttctccaaaatactcacatgttcattacttatatatcgttttctttttaatactaaaggtcaacccatctggtacagtgaaaggttttatagctgcaagtttaagtaccaacaatcccgattacttttactcctggactcgtgacagtgccttagtggctcatacaatgacatatctttataatacatcggaagccggtgactctactatcgaaagtgcattgaaggattatgttacattctctatcaacgctatgaatgctgctactgtttgtaattgtttaggtgaaccgaagttcaatactgtaagtataaggggaaaaaagagacgatactttaaaaatgatagacttatcattggatgaaactttttttttaacaaataggatggaagtggttatactggagcatggggaaggtaggttttttatttacatgctggctccagtgcttaaatgatatgattatttgggagtgaaaaataaatgttgtgagcaaccttgctaaacttgtttctattattattagaccacaaaatgatggccctgcttcaagagcaacaaccatgattttgtttgctgatagtttccttgctcaaggaggtgatgtttcctatgtcatcaacacactgaagccaggtatatactattatatgtcccaactacatttattgaaatataaagtactaatcttaacaaatattattatttatcttgcccaatgatatttagccatctataaagatcttgactatgttgttagtacctggtccaatacttgttatgatttatgggaagaagtcaacggtgttcatatctgtaagtagtagtatatctcttaaataatgcatcattctgaatatacatatttatcttcaattttttttttaaatagatacattatccgttatgcgcagagcactgattgatggagctaactttgctcaacgtaatggtgatacttctcgcgtgagcggttatactagtactgcaaccacaatcaagacacgtctcgaatccttctggtcagatagcaataattatattactgtcactcaatcctattcaggtggtgttcaaaaggctggattggatgtctctactttgattgcagccaatatcggaagcgtgggtgatggtatatttcatctattactactttatttccatattattgcgttatcggatcgatcttctctgtcattttataacgtaatatttattaatatctcttttctaatatttataataaggtttctatactcctggttcggataaagtcttggctactgctgttgctattgaaaaatcatttgccaatctttacaccatcaaccaaaacaagccttcatggcttggtaatgctattggtcgttatcccgaagatacttatgatggttatggtaattccaagggtaatcccgtaagtatttttttttatctcttccgtttatacttttaatgcagataatgatgatatcatcactatgtccggtcgaatagtaatccatttgttgtatatagtggtttattgctactgcaacttatgccgaattatactatcgtgccattttggaatggcaacaacaagcttctgttactgtcaactctatcaatcttggcttctttagcaagtttgattcctctgctagtgtgggtacagtctatacacctggtacagatagctttgccaacatggtatccaatgtcgcctttgctgccgacgaattcttgtccaccattgactatcatgccatgaacaatggatccatgcatgaacaatacaatagagatactggtatctctcaaggtgctcgtgatttgacttggagtcatgctgctttcattacagctgccaaagccaaattaggtgccccagcattttaa

SEQ ID NO: 15 - DNA, 2033 bp, Rhizopus microsporus
  source 1..2033: mol_type = other DNA
atgaagttaatgaactcgtcgatgaagacctgtgtcttctttattctctcctacttttctttgttggtctcttctgctgcagtccctactagtgctgctgtacaggttgaatcttacaaatatgatggcactaccttctctggtcgtatctttgtaaggctaattgatctcatttaaagaaaaagagagaaactgatctcgtcaattaggttaaaaacatcgcctactcaaaggtagtcactgtcatttattctgatggctctgataactggaacaacaacaataacaagatttctgctgcttactctgaagctatctctggttcaaattacgaatactggacattctctgccaagttgagtggtatcaagcaattctacgtcaaggtaattctttttttttctctctatttattgtgtatactaacgcgacctttgatagtatgaagtaagtggctctacctactatgacaacaacggtacgaagaattatcaaggtaagcatatgcttaagcaatagcatttctttactgacctatcaaatagtccaagcaacaagcgctacctctactactgctactgctactactactacatccaccagcactactaccactagcactggcccaacaagcactgcctccgtctctttccccactggtaactctactatctcatcttggattaagaaccaagaagaaattagccgttttgccatgcttcgtaacatcaatcctcccggaagtgccacaggatttattgccgcttctttatctaccgctggaccagactactactattcttggacccgtgattctgcattgacagccaatgtgatcgcatatgaatacaacaccaccttcgctggtaacaccacccttttgaaatacctcaaggactacgtcacattctctgtcaagtcccaatccgtttctactgtctgtaactgccttggtgaacccaaattcaacgctgatggctctagctacactggtccatggggaaggccccaaaacgatggacctgctgagcgtgctgttacttttatgctgattgctgacagctaccttactcaaacaaaggacgcttcctatgttaccggtaccttaaagccagctatctttaaggatttggattatgttgttagcgtatggtcaaatggatgttatgacttgtgggaagaggttaatggtgttcatttctacactttgatggttatgcgcaagggtttgattcttggtgctgattttgctgctcgtaatggtgactcttctcgtgcttctacctacaagaacactgcttccacaatggaatctaagatatccagtttctggagtgactccaacaactatgttcaagtcagtcaaagcgttactgctggtgtcagcaagaagggtttggatgtttctactctgctggctgcaaacattggtagtcttccagatggttttttcactcctggttctgaaaaggtatttgcaaaaagtcgctcaatatttcatagattgtaaacgtgattttatctagatccttgctactgctgtcgctcttgaaaatgcctttgcttcattgtatcccatcaactctaaccttccctcatacctcggtaactctattggaagatatcctgaagacacatacaacggtaatggtaactctcaaggtaacccatggttcttagctgtcaatgcctacgctgagctctactaccgtgccatcaaggaatggatcagcaatggtaaggttactgtgagcaacatcagcttgcccttctttaagaaattcgactcctctgctacttctggaaagacttacactgctggcacttctgactttaacaaccttgctcaaaacattgccttgggtgctgatcgcttcttgtccactgtcaagttccatgcctataacaatggctctttgtctgaggagtatgatcgctctactggtatgtccactggtgccagagatttgacttggtcccatgcctctttgatcactgctgcttatgcaaaggctggctctcctgctgcttaa

SEQ ID NO: 16 - DNA, 1469 bp, Spinellus fusiger
  source 1..1469: mol_type = other DNA
atgaagtcaccttatagcatgaaaaccgttttggcattgttgtctcttaccccgcttttcacgtcagatgttgttgctgtgccttctggtaactccaccatcactgcctggggaagcaagcaggacggcattagtttctccaccatgcttggtaacatcaaccctcctggatctagcaagggattcattgctgcctctctctctactgctggtcccaattactactactcatggacccgtgactctgctttggtagctcgtgccatcacctacaaatacagcacttcttaccagaacgaccccaagattctcggtctcttgaaagactatgttacctaccaggtgaacgaacaaacagaaagcaccgtctgcaactgtctcggtgagcccaagttcaaccccgatggttctagcttcagtggtccatggggaagaccccagaatgatggtcctgctgaacgtgcctccaccatgattctgtttgccaagagttactatgctcagaccaatgatgttggctatgtcagcaatacactgaagcctgccatttacaaggacttggattacattgtgaatgtgtggggtaacaactgctttgatctctgggaagaagtcaatggtgtccacttctataccttgatgatgatgcgccatggtcttgtccagggctctatctttgctaacactctcggtgattctaccagagccaacacctacaagacagctgcccagaacatcaagaacagaattgatactttctgggaatctggaagcaactacattgtcgtcactcagaaccagagtgcaggtgttaacaagccctctggattggatgttgctgttttgcttgctgccaaccaaggtggtctgggagatggtgtctatactcctggctctgacaaggtaaatgattatctgctggtctttttagacacaccaaactcatccattttatatgataggttcttgctactgctgttgctcttgagaaatcttttgccagcttataccctatcaacaagaaccttccttcttactacggtactgccattggaagataccccgaagacacctacaatggcaatggcaactctgaggccaacccatggttcattgccaccaccacttatgctgagttgtactaccgtgctatcagtgaatggaccaatggctcaggtgtcactgtaaacagcatcaacaaggagttcttcagcaagtttgatgccagtgccaccaacggaaaggtctacacacctggatccgatagcttcaacagcctggtcaacaacgttgccattgctgctgataacttcctttctactgtgagataccaccaaaccagcaatggctctttatctgaacagttcaaccgctacactggattcatgacaggtgcccgtgatcttacctggtcccatgctgccatggtcactgctcttgctgccaaggccggtactccttccccttaa

SEQ ID NO: 17 - DNA, 2402 bp, Syncephalastrum racemosum
  source 1..2402: mol_type = other DNA
atgcagctaaacctttactggaaaagtctggccgtgctcgccctgattcagatggtcatggcggccactgtgccaaccactcaggttcagctagactactacacgtactccaacaatgtactgtcgggccgcatctatgtaagccgagatagaccaggaaacgccgctgaaagagtgctcattacatatatatcttttcttgcaggtgcaaaatattgcatactcaaaggtggtcaaagtgatttactcagacgcgtctggaaactggaacaacaacggcaatactatctccgcaagctatgtcgagagtatctctggtaccaactatgagtattgggatttctcggccaccattggcacggctggtatcaagcaattctatcttcgcgtgagtttgtcacctgccttgtctcttattattaagccattaggctcgagtgacattaatgctgtatttcatgtgcatattagtatgacgtatcaggtagcacttactatgacaacaatggtggggataataacaactacagtacgtattatcggacagcgagagtcacttagccacttatgtaacatattacacatatagacgtcgtcgcgacgacttcaacaacctcgtcatccacaaccacgactacggctacaacgacaaccgccacaaagactactagcaccacaagtgccacagctacgcccacgtcatctgcgacttttcctagcggcaattcaacgatcaccacttgggcaaaatctcaaaaggatattagctggaagacactgctcaccagcctgaatccctccggcactgccaaaggcttcattgctgcatcgctttccacatcgaacccagactattattatgcttggacccgcgactcggccctggttgccaggttggtcgactccatttattgcacttgggcaagcacccttcttacgcgacgaattgatggttagaaccatggtcaacatgtacaacaccacggaagctggaagtgcgtcggttctaggccttttgcaagactatgtcaccttccaaattaatgccatgagcacttctacggtatgcaactgccttggtgagcccaagttcaacccggatggctcgagttacaccggtgcctggggccggtaattgcaaaatcacgacggtttgcgtttcgtaatggttgatttctgtgtttgtagtccccagaacgatggccccgctgaacgtgccagtaccttcattctgtttgccgatagttatatcgctcaaggtggccagctctcatatgtgacaggaactctcgctcctgctatctacaaggatctcaactatgtcgtgagcacctggtcaaataactgctttgatctttggtatgatattggtcgatattgcatatttccgtcacgtatttctaactcgttctaatatagggaggaagtcaatggccgacatatgttcacgtaaggaaggatatgcgctaacgttcctttctcagatttgatctcatttcttgtagccttgctgtcatgcgccgtgctctcttggatggtgtcaacttcgcttcgcgcatcggcgatacgacttactcgagcacgtggtccagtaccgcatcgtctatccagtcaactttgtccggctactatttgtcgagcggcaattacattactacggtgcagaacttccagtcaggcgtcagcaaagccggttatgacgtctcgactttgattgctgctaatgtggccggtatgggcgatggcttctttacgcctggctcggacgaggtgaaccatcaaaccgcgtaaacttcgtagtagaagactgaccgtggtgtaggtactcgccactgctgtggctatcgagaacaaattctccagcttgtacggcgtcaacgcgaacaaagcttcttatctcggcactgccatcggacgctacccagaggatacctataatggctatggtaacggccaaggaaacccggtaagtattactgcacttttcgtttgtactcaaacatgcagaaccgcgatctaaatggcaggtccgtatgtagtggtttattgcaacggcggcatatgctgagctgtactatcgtgccattttggagtggcagaccaagtcctccatcgtagtcaactccaaaaacctcggtttcttctccaagtttgattcatccgctgcagtgggtactacctatacgcctggcaccacagcttacagcaacatggtgcaaaatgttgccttggctgccgaccggttcttgtcgaccgtacagctccacgccgctaccaatgggtccatgagcgaacagttcaatcgtgatacaggcgttatgcagggtgcacgggatttgacttggtctcactctgctttcattaccgctgcgcgcgccaagcttggttcgcccgtctactag

SEQ ID NO: 18 - DNA, 1827 bp, Syncephalastrum racemosum
  source 1..1827: mol_type = other DNA
atgcgcgctacaacagctattctgtctctctttaccactgcgtccatggcgctggccgccaacagcactgtcccggatggcaactcaaccacgaccgcctgggtcaagaagcaagaagccatctcttggacagatctcaagaccaatgtcaaccctgaaggtgcggccaagggctttattgctgcgtctctttccacatcggagccagattattactatgcttggacgcgtgattctgctcttgttgcgcggtatgctcaacgtgacatttacaatgcctaatactgacataataatattcagcgtcatggtcaacaagtacaacacgaccgatgcgggtgacgccaacctgctaggtcttttgcaagactatgtgtcgtttcagatcaatgccatgggtgaaagcactgtctgcaactgcttgggtgagcccaagtttaacccggtaagaagacaaaaaacataagacagaaaagaacaatcggctgattctgggaccttgttacacacaggatggctctagctacactggagcctggggccggtgcgtatttgattagatgctcctgtaccacgttaatgttaatatttgcccacatatagccctcagaacgatggccccgcggagcgtgcttcgacttttatcttgcttgctgatagcatgattgctcaaaagtcagcgaatggttcttatgtctcggataccttagcccctgccatttacaaggacttggcctatgttgcctccacctgggagaatgcttgctacgatctttggtaaggctattccaatgcaaattatgtctgctagcttcatttctaacgaaccggttctttaatcttttagggaagaagtcaatggcaagcacatgtacacgtaagtactatatattctttgaatgattattagtaaagtatatatatgactaatgatgctttgtctttgtatcgctagcctgtccgtgatgcgccgcgctcttctagatggtgctgattttgctagccgccagggtcagacggcgaatgtgacctcgtggaagtcaaccgccgacaagatcaagtcttcgctcgagggtttcttctcatccgataatggttacattgaggtgacccaagatatgcagggcggtgtccaaaagaagggcctcgatgtgtcgaccttgatcgcagccaatattggcagcatgggcgacggtaatatcttcttatgcgagctatatgcctgcgctcaacaacattattaggcttctacaccccgggctcagacgaggtgctagccactgccgtggctgtcgaggctgcgttcgctgacctctacaagatcaatcagaacacgaactccaccggccttggtaccgccatcggccggtaccccgaagacacttacaatggtgtcggcaacagccaaggcaaccccgtaagtcactatcatctttgttgattatctattttaacttctattagtggttcattgcaaccaacacgtttgctgaactctactatcgcgctattcttgagtggcagaacaagggtacaatcacggtcaactcggtcaacgcggccttcttctccaagtttgattcctcggctaaggctggcactacatacaagtcaggcagcactgaatttgacagcttgatcaataaggtggctttggctgccgatgccttcttgaacacagtccagacgtatgctgcaagcaacggctcgatgagtgagcaatacaaccgtgacaccggtgcgctgacgggtgctcgcgaccttacttggtcacacgccagcttgatcactgctgcaaacgccaagctcggtactccttataactag

SEQ ID NO: 19 - DNA, 1796 bp, Thermomucor sp.
  source 1..1796: mol_type = other DNA
atgaagcttggccgtatcggattcagtatcctgtcagttgcctccgtcttttctcaagctgttgttactgctgctgctgctgcttcggtaccctccggaaacgcgaccatcacgtcgtggatccaagagcaactggccattagctggaacaccatgctgacgagcgtcaatccggttggcgccgtgaccggcttcattgccgcgtcgttatcgaccgcgaacccggactactactactgctggacgcgcgacgctgcgctggtggcacgcgtcatgacgttcatgtacaacaccacccaagccggcgacagtggccttttgggaatcctgcaagactatgtgtcgttccagatccatgccatgggtgaatccacggtttgcaactgccttggtgagcccaaatttaatccggtaaatgcgcggccaatcccccaaactttctatacgtatatatattggttcaagctgaacgatggtttaggatggttcgagttacactggcgcatggggccggccccagaacgatggacctgccgagcgggcgtcgacgtttatcaaaattgcagatagttaccttacccaaaccggtgacgtttcgtacgtcacgaatacgctgaaaccaggtgcgtatatatatatatatatatatgtatatatatatagaacaggatacaacaaaaggtagtagacgaagaactcaactttttttcggacaaagcgatctatgaggatctggactatatcgtgaatgtttggcaaaatacgtgctttgatctgtgggaagaagtctatggcatgcatatgtacacgtaagttgtattctctctattatgtccattcctgtggatcttcgtacttatgatgtatgtccatgtaaagattggccgtgatgcgccgcgggttactggacggcgcggatttcgcgacgcgcaacggcgacaccgacaaagccagcacctacaccagcacggcaacgtccatccagacccggctcgcgacgttctggtcggatagcaatggctacattactgtgacgcaagattaccaaagtggggttagcaaagccggacttgacgtctcgactctgattgctgcgaatgtggccggcatgaacgatgggttctttacgccgggctcggatgaggttcgtgttatacatacttgtacaccaaggaggatgcctattgacaaaaaaagcgcggttgttctagatacttgctacggctgtcaaggtggaagctgcgtttgcaaacctttacggtatcaacatcaacaaggccagttatcttggtaatgcaattggtcggtatcctgaagacacgtataacggcaacggcaactcggaaggcaacccggtaagctatcccatttttgtcccccgatttctaagtttgcaactttctctaaccaatgttgcttggtgttaaaagtggttcattgccacagcggcgttcgcggagctttactaccgggcgatcatggagtggcaattgcgcggcagcagcatcacggtgaactcggtcaaccagggcttcttcaccaagtttgatccgtcggctacggctggcacgacgtatacgccaggcacggacgcgtttaactcgctgattgacaatgtggcgctggcagccgatcagttcttctcgaccatccacctgcaccgtgcgacgaacggctccatgagtgagcagtataaccgcgatacggggttcatgcaaggcgcgcgggatctgacttggtcccatgcggcgtttatcacggccgccaaggcgaagcagggcaccccgtcattctag

SEQ ID NO: 20 - DNA, 2883 bp, Zychaea mexicana
  source 1..2883: mol_type = other DNA
atgagaccagttcatctttggaaaagcctgcttttagctgcggctactgcagtcacgggtacaatagctgttgatgtaccttcggtgcctattcagttggagtcctatacttattccgaaaatgtattcgctggtcgaattttcgtaagttatggaagccataataataaagtggcaggatctatgatgcacaataactaaactctctgattactcgttaactccctttgtattttgatatctaggttcaaaacattgcgtacaccaaagaagtcaacgtgttctggtccgatgcttctgatgactggaacgacaatggcaactctgtggcagctagttattccgaatccatcgcagacaccaactatgagtactgggaattttcaaccacgattggatctgccggtatcagccaaagttatttgagagtaagtcccaatcgccattttatatatattgtgcacgtcagcaatagagctctgcagtaaagatataacttcagataaccttgacatctatgtctgaacaatttcgggttttagtatgacgtatctggatcaatctattatgacaataacgacagccagaactacggtaagcagagtatgagctacatctttgaagccttcataatgaagcagctgcacatatactgcctcctaattgggttgcacgtacaacgggaaggattattgctaactttatatgttacgtttgtaccagatatcaccgagacatcgacccctaccacaaccactgctgctcccacttctaccactagcactcctactactactacggatggtggatcgacaactactaccactgcgacatctgttcctacgtctactggtgttccagagggcaatgccaccatcagcgaatgggcaagcgctcaattggatatcagctggccaaaccttatgatgaatgtaagtcaacaatactctgtatagtgcagcatacattacaagccattcaattaatcgaattttcttacattctctttttttgttcccaggtaaacccatcaggtgcagtgactggctctatcgttgcctctttgtcgactagtaacccagattatttttacatttggacgcgagatgctgcaatggttgcacgcgtgatggtatacatgtacaacacaaccgaagctggtgacacaaatctgttaaatgccttgaccgactatgttacgttctccatcagctccatgaatgtggataccgtctgcgactgtttgggcgagcccaaattcaatgtggtaagtagagaaattttataaaaacagggctaagtgatactgaagagcaaggataaaaaagtgaggacaaaaagagtagcaatactagtgttaataatatacgattcattataggatggaagcggttacaccggtgcgtggggaaggtacagtaattatggaactagaggacagaaaaggctgtattttttgtattatgtgatatgcactgaaaggttggatgaatcttttgtttgcaacaatgtactcacattcatttatgcattagaccccaaaatgatggacctgcagagcgtgcttcaaccatgattttgatcgcagacagctatatcgctcagggcggtgatgtttcctatgttactgacacgctgaagccaggtatgtaaattttctatactttactcaccatcagcaacacggagttttcctgtcatagataacaaacgatcattgacacattatggtctgtgatcttttttatagccatttacaccgatcttgattatgttgttgatacttggtctaacgtttgcttcgatctttgggaagaagtgaatggcattcatatgtgtaagctatttgtgataatcatttttatatgatgtaaaaggaaagtctcttggctcactagtatgatgaaacatagacaccttgtcggtgatgcgcaaggctcttctcgatggcgccaattttgctacccgcaatggcgacacctcgcgtgtcagcggttatgaaagcaccgcttcatccatcaagaccagactcgaatcattctggtcctctagcaacaactacatcaccgttacccaatcgttctcgggaggtgtttcaaaggccggcttggatgtctctacattgattgctgccaaccttgcaagcatgaacgatggtaagtgattacttcacatgaaaaaggtgtatatttttactcacatatgtctgtcccttgatggttgtaatttaggtttctatactcctggctctgatgaaatcttggccacggctgtcgctattgaaaactcttttataagtgagtatacgctcaatcaaaacagaccatcgtggctcagcacagctattggccgatatcctgaagattcttatgatggctatggtaactcgcaaggcaatccagtaagtaaaaagagtagttcctttacgtaacgctactactcgtagtgatgatgtgcacaagagcagcaaatccaattcagctgtagttcgattactgacaatgcaattatgtcctctagtggttcattgccactgccacatatgctgagctctactaccgtgccattttggagtggcaacaacaagcatccatcagtgtcaactctgtcaacctgggcttctttagcaagtttgattccgatgcttctgtcggcactgtgtacacaccgggcactgaagattttgccaacatggtctccaacgtagccttcgctgctgacgaattcttggctaccattgagaaccattctgccgttaatggatccctctctgagcaatataacagagatactggtattatgcaaggcgccagagatctcacctggtcccatgccgcatttattactgctgcgaaggcaaagcagggtgcaccaatacactga

SEQ ID NO: 21 - DNA, 1407 bp, synthetic construct
  misc_feature 1..1407: note = .
  source 1..1407: mol_type = other DNA
atgccttcttggaaaaccctgttcctcctcctgggccctatcgccaccgctgccgctgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagtctaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttctaa

SEQ ID NO: 22 - DNA, 1818 bp, synthetic construct
  misc_feature 1..1818: note = .
  source 1..1818: mol_type = other DNA
atgaagctgttcggcaccctgaaggcttctttcgttttcctgattggctgcattgttttcgctatggccgacaccgtccctaccacccaggttaagctgcagtcttacacctactctggcggcgtcctgtctggcaccatctacgtccagaacattgactacaccaaggttgttaccgttatatattccgacggctctggcaactggaacaacaacggcaacaccattagcgcttcttacagcagctccatttctggcaccaactacgagtactggaccttctctgccagcgtcagctctattcagcagttctacatctcttacctcgttgacggcaccacctactacgacaacaacggcggctacggcgctaactacgccgttaccgccaccaccaccaccgcttcttccaccaccaagaccacctccggcaccaccaagacctccaccaccagcaccgctaccgccaccagcaccggcacctcttccttccctagcggcaactctaccattagcacctggagcccttcccagcacagcattagcctgtacgccatgctgcgaaacattaaccctcccggctctgctgctggcttcatctctgctagtctgtctacctccggccccgactactactactcttggacccgagacagcgccctcgttgctcacgtcatcgttaacgagtacaacaccacctaccagggcaactccaccctgctgggcatcctcaaggactacgtcacctacagcctgaacgcccagaccacctctaccgtttgcaactgcctgggcgagcctaagttcaaccccgacggctcctccttcaccggcgcttggggccgccctcagaacgacggccccgctgagcgagctgtttctttcatatatttcgctgactcttacctgacccagacctctgacagctcttacgtcaccggcaccctcgcccccgctatatacaaggacctcgactacgttgtttccgtttggtctaacggctgcttcgacctgtgggaggaagttaacggcattcacttctacaccctcatgttcatgcgacgaggcctgctcgacggcgctaacttcgcaagccgaaacggcgactctacccgagcgagcacctacacctctaccgctgctagtatcaagaccaagattgacggcttctgggtcagcagcggcaactacgtccaggtttcccagtctgttacctctggcgtctctaaggctggctacgacgcttccaccctgattgccgctaaccaggcctctcggggcgacggcttctacacccccggctccgacaagatgctcgctaccgccgttgctattgagtctcagttcagcagcctgtactctattaacaccaacaaggcttcttacctgggcaacgccattggccgataccccgaggacacctacaacggcaacggcaactcccagggcaacccttggttcctgtgcaccaacgctttcggcgagctgttctaccgcgccattagcgagtggaacgccgctggctccgttaccgttaacagcgttaacctcgctttcttcaagaagtacgactctagcaccagcagcggcaccacctacaccgttggcaccagcgcttacaacaacctggttcagaacgttgccctcgctgccgacgcttacttctctaccgtcaagtaccacgccctgaccaacggctccatgtctgagcagtacgaccgatctagcggcatggctaccggcgcaagagacctgacctggtctcacgccgccatgattaccgccctgaaggccaagtccggcacccccgtttactaa

SEQ ID NO: 23 - DNA, 1809 bp, synthetic construct
  misc_feature 1..1809: note = .
  source 1..1809: mol_type = other DNA
atgaagcctttcggccctatcaagaccaccctgttcattattatctctcacttcagcctgaactccctcgctgacaccgttcctaccacccaggttgaggtcaagacctacacctactctggcggcgagctgtctggcaccatatacgtagagaacatagactacaccaaggttgttaccgtcatctacgctgacggctctgacgactggaacaacaacggcaacaccattgctgccgctttcagcgagtccatctctgacaccaactacgagtactggaccttctctagctctgttaactctattaaagagttctacgtcaagtacgacgttgacggcaacacctactacgacaacaacggcaacgctaactaccaggtttctgctagtagcaccaccaccaccaccgccaccaagaccaccaccaccgctaccaagaccaccgccaccgctaccagcaccgccaccggcagcacctctttccctagcggcaacagcaccatcagctcttggattaagggccaggaagacacctcccggtccgtcatgctcggcaacattaaccctcctggctctgctaccggcttcattagcgccagcctgtctacctctggccccgactactactaccactggacccgagacgctgccctcgttgctcacgttattgttaacgactacaacaccaccctgtctggcgactctagcaccctgcaggtcatcaaggactacgttaccttcagcgtcaactcccagtccgagtctaccgtttgcaactgcctgggcgagcctaagttcaaccccgacggctcctcttacaccggcgcttggggccgcccccagaacgacggccccgctgagcgagctaccaccttcattttcttcgccgacacctacctcgcccagggcggcgacagctcttacgttaccggcaccctcgcccccgctatatatgctgacctggactacgttgttaacaactggtctaccggctgctacgacctgtgggaagaggttaacggcattcacttctacaccctcatggttatgcgccgaggcctcatagatggcgcttccttcgcttcccgaaacggcgacagcacccgaagctcctcttacacctccaccgccaagtctattgctaccaagattgactctttctggtctgcttctaacaactacgttgctgtttcccagagcgttacctctggcgtctccaaggctggctacgacgcctctactattattgctgctaaccaggcgtcgctgggcgacggcttctacacccccggctccgacaagatgctcgctacctccgttgccgttgagaacgctttcagcagcctgtacaccgttaacaccggcaaggcttcttacctgggcaacgccattggccgataccctgaggacacctacaacggcaacggcaactctcagggcaacccttggtatctgtgcaccaacgccctgggcgagctgtactaccgcgccatcaaagagtggaacgctgcgggaagcgttaccgtcaactccgttaacctgggcttcttccagaagattgactccagcattagcagcggcaccaccttcaccctgggcacctccgactacagcaccctcgttgacaacgtcgccctcgccgctgacaagttcttcgccgtcgtccagtaccacgagcgatctaacggctccatgcctgagcagttcggccgagaggacggcctgcctaccggcgcgagagacctgacctggtctcacgccgccatgattagcgctgcccgagctaaggctggcacccccgtttactaa

SEQ ID NO: 24 - DNA, 1806 bp, synthetic construct
  misc_feature 1..1806: note = .
  source 1..1806: mol_type = other DNA
atgaagttctctcacctgttccgaaagcctaccctcctcattgttaccttcctgaccgccaccgttttcgccgagaccgtccctaccacctcccaggttaagctcaagtcttacacctacgacggctctaccctgtctggccagatttacatccagaacattgcttacaccaaggttgttaccgtcatttactctgacgccagctctaactggaacaacaacggcaacaccattgctgcctcttacaccaacggcattagcggcaccaactacgagtactggaacttcaccggccccgtcagctctattaacgccttctacattaagtacgacgtttctggcaacacctactacgacaacaacaacaacgctaactaccaggttaccaagacctccaccaccaccaccaccagcaccgccaccgctaccaccaccaccaagacctctacccctacctccaccggcgttccttctaacttccctaccggcaactctaccatcagcacctggctcaagtcccagattggcatctctcgatacgccatgctgcgaaacattaaccccgctggctctgccgttggcttcattgctgctagcctcagcaccgctaaccccgactactactacgcttggacccgagacagcgccctgacctcttacgtcatcgctaacgactacaactctaccctcgctggcaacagcaccatcctgcagattatgaaggactacgttaccttcagcgtcaagtctcagtccgtttctaccgcttgcaactgcctgggcgagcctaagttcaaccccgacggcagctcttacaccggcgcttggggccgcccccagaacgacggccccgccgagcgagctaccaccttcatcctgttcgctgactcttacctgaagcagaccggcgacgcttcttacgttaccggcaccctgaagcccgctattttcaaggacctcgactacgttgttaacacctggaccaacggctgcttcgacctgtgggaggaggttaacggcgtccacttctacaccctcatggtcatgcgaaagggcctgatccgaggcgccaacttcgctacccgaaacggcgacagcacccgagctagcacctacaccaacaccgctgctagcatcaagaccaagatggactctttctggagctctggcaacaactacattgctgtctcccagtctgttaccggcggcgtctccaaggctggctacgacgttgctaacattattgccgctaacgttggcagcctgcaggacggcgtttacacccccggctccgaccgcattctcgccaccgccgttgccgttgaggctaagttcgccagcctgtacggcgttaactctaacctccccggctacctgggcaactctattggccgataccctgaggacacctacaacggcaacggcaactcccagggcaacccttggttcctcgccaccaacgcttacgccgagctgtactaccgcgccattcgcgagtggtacgacaacggcggcgtcaccgttaactctgttaacctccctttcttcaagaagttcgacagcagcgctgctagcggcaccacctacaccgttggcaccaccgctttcaacaccatggtttctaacgttgctgctgccgctgacaagttcttcagcaccgtcaagttccacgcttacaccaacggctccatgtctgagcagttcggccgaaacgacggcctgtgcaccggcgcaagagacctgacctggtctcacgctagcctgattagcgctgccctcgccaaggctggcacccctagcgtctaa

SEQ ID NO: 25 - DNA, 1797 bp, synthetic construct
  misc_feature 1..1797: note = .
  source 1..1797: mol_type = other DNA
atggtttctttcaacttcctgaagaagcctgttttcattgttatcacctgcctcaccgtcagcgtttgcgcccagtctgttcctaccagcgaccccgttaaggttaagaccttctcttacgacggcaactctttctctggccagatttacatcaagaacattgcttacgagaagaccgttaccgttatttactccgacggcaccggcaactggaacaacaacaacaacaagattgccgctgctttcagcgaggccatctccggctctaactacgagtactggaccttcagcgctagcgttcctagcatcaagcagttctacgtcaagtacgacgtttccggcaagacctactacgacaacaacggctctaaggactacaacgtcgttaccagcggccctaccaccaccagctctggccctagcaagaccaccaccaacggccccgctcctaccagcaccaacaccaacttccctagcggcaaccctagcattacctcttggattgacaagcagattgacatctcccgatctgccatgctgaagaacattaaccccgctggcaccgttaagggcttcattgccgccagcctcagcacctctaaccccgactacttctacgcttggacccgagacgccgctctcgttgcccacgttgttgctaacgactacaaccgaaccaagtctggcgacgccacctacctgggcctcctgaaggactacgtcaccttctctattaactcccagaacacccctaccgcttgcaactgcctgggcgagcctaagttcaacaaggacggctccggctacaacggcccttggggccgccctcagaacgacggccccgccgagcgagctgacaccttcgtcctcattgccgactccattctgacccagaccaaggacgtctcttacgttaccggcaccctgcgccccgctatttacaccgacctcgactacgttgtccgaacctggtctaacggctgcttcgacctgtgggaggaggttaacggcgtccacttctacaccctcatggttatgcgacgagctctcctcgttggcgctaacttcgctagccgaaacggcgactctgcccgagcttctaactacaacaacgctgctaactccattaagtctaagattgactctttctggtcctctaacaacaactacgttgctgtttcccagtctgttaccggcggcgtttccaaggctggctacgacgtcagcaccctcatcgctgctaacgttggcagcctgtctgacggcttctacacccctggctctgagcgcatgctcgccaccgccgtcgctattgaggacaagttcgctaacctgtacggcattaaccgaaacctcgacagcagcctgggcaacgccattggccgataccccgaggacacctacaacggcaacggcaactcccagggcaacccttggttcatcgccaccaacgcttacgctgagctgtactaccgagctattaaggagtggaacaacaacggcggcgtcaccgtcaccaacgttaacctgaacttcttcaagaagttcgacggcagcgctagcgttggcaccaagtacaccgctggctctgctgcttacaacaccctgacccagaacatcgccctcgccgctgacaagttcttcaacaccgtcaaggtccacgctgccaccaacggctccatgtctgagcagtaccaccgagacaccggcagcatgaccggcgcaagggacctgacctggtctcacgctagcctgattaccgccgccctcgccaaggctggcacccccgtcgcctaa

SEQ ID NO: 26 - DNA, 1830 bp, synthetic construct
  misc_feature 1..1830: note = .
  source 1..1830: mol_type = other DNA
atgaagctgttcggcctgcccctcaagcctagcctgttcttcgtcgtctcttacctgtctttcctcgcttctgctgccagcatccctagcagcagcgaggtccagctcgacacctacgcttacgacggcgccaagttctctggcaagatttacgtcaagaacattgcttacgagaagaccgttaccgttgtttacgctgacggctctgacaactggaacaacaacggcaacattattagcgctaccttcagcacccctatttccggctctaactacgagtactggaccttcagctctagcattagcggcattaaggagttctacatcaagtacgtcgtttccggcaagacctactacgacaacaacggcaccaagaactaccaggtttccaccaccagctctaccaccaccgctagcaccaccaccgctacccgaaccaccaccgctggcaccaccagcacctctaccaccgctgcccctacctctacctccagcggctccttccctagcggcaactctaccgttagctcttggattaagcgacaggagaagatctcccgattcgccatgctgcgaaacattaacccccctggcagcgctgctggcttcattgctgctagcctgtctacctctggccccgactactactactcttggacccgcgacagcgccctgacctctaacctgattgcttacgagtacaacaccaccctgtctggcaacaccaccattctgaacatcctgaaggactacgttaagttctccatcagctcccagaccaccagcaccgtttgcaactgcctgggcgagcctaagttcaaccacgacggcagctcttacaccggcgcttggggccgcccccagaacgacggccccgctgagcgagctaacaccttcatcctgttcgccgactcttacctcgaccagaccaaggacgcttcttacgtcaccggcaccctgaagcccgctattttcaaggacctcgactacgttgttaacgtttggtctaacggctgctacgacctgtgggaggaggttaacggcgttcacttctacaccctcatggtcatgcgaaagggcctcctgctgggcgctgacttcgctaagcgaaacggcgactctacccgagctagcacctacaccaacaccgctagcaccattgctaccaagatctcctctttctgggttagcagctctaactggatccaggtttctcagtctgttaccgccggcgtttccaagaagggcctcgacgtcagcaccctcctcgctgccaacctgggctccgttgaggacggcttcttcacccccggctccgagaagattctggccaccgccgttgccattgaggacgctttcgctagcctgtaccctattaactctaacctgccttcttacctgggcaactccattggccgataccccgaggacacctacaacggcaacggcaactcccagggcaacccttggttcctcgccgttaacggcttcgccgagctgtactaccgagctatcaaggagtgggttaacaacggctccgttaccgtctctaacatctccctgtccttcttcaagaagttcgacagcagcgccaccgccggcaagacctacaccgccggcaccgctgacttcaacaacctcgcccagaacattgctctgggcgctgaccgattcctgtccaccgtccagacccacgctttcaacaacggctccctcgccgaggagtacgaccgaaccaccggcgtcagcaccggcgcccgagacctgacctggtctcacgctagcctcattaccgctgcttacgccaaggctggcagccctgccgcctaa

SEQ ID NO: 27 - DNA, 1827 bp, synthetic construct
  misc_feature 1..1827: note = .
  source 1..1827: mol_type = other DNA
atgaagttctctaacctcctgaagaagcccctcctgctcattgctggcattctcgctgccaccgtcgttgccgagaccgtccctaccaccgctgctgttaaggttaagtctttcacctacgacggcagcaccttcgctggccagatttacgtcaagaacatcgcttacaccaagaccgttaccgttatttactctgacgcctctaacaactggaacaacaacggcaacaccattgccgcctcttacagcgagggcatctctggcaccaactacgagtactggaccttcagcgcccctgtttccggcatcaagcagttctacgtcaagtacgttgtcagcggcaccacctactacgacaacaacaactctggcaactaccaggttaccaccaccaccaccaccaccgcccctaccagcaccacctccggcggctctagcaccaccaccggcggctccaccaccaccgccacctctgtccctaccggcgtccctagcggcttccctaccggcaacagctctattagctcttggattgacggccagacctccgtttctcgatacgccatgctgcgaaacattaaccccgctggcgctgtttccggcttcattgctgctagcatgtctacctctggccccgactacttctacgcttggacccgagacagcgccctgacctctcacgtcgttgcttacgactacaacaccaccctcgctggcaactctaccatcctgggcctcctgaagaactacgttaccttctctattaactctcagaccaccagcaccgtttgcaactgcctgggcgagcctaagttcaacaaggacggcagctcctacaccggcgcttggggccgaccccagaacgacggccctgctagccgagctgacaccttcatcctcattgccgactctattctgaagcagaccggcgacgctacctacgttaccggcaccctcgcccccgctatttacaaggacctcgactacgttgtcagcacctggtctaacggctgcttcgacctgtgggaggaggttaacggcgttcacttctacaccctcatggtcatgcgacgaggcctgattaagggcgctaacttcgcttcccgaaacggcgacaacacccgagctaacacctacaccaacaccgccgctagcatcaagaccaagattgactctttctggaactctaacggcaactacgttaccgtttcccagtctgttaccggcggcgtctccaaggctggctacgacgctagcgtcctgattgctgctaacctgggctctgtccaggacggcttctacacccctggctccgacaagatgctcgctaccgctgttgccattgagtctaagttcgccagcctgtactccattaaccagaacctgaacggctacctgggcaacgccattggccgataccccgaggacacctacaacggcaacggcaactcccagggcaacccttggttcatttgcaccaacgctttcgccgagctgtactaccgcgccattaaggagtggttcaacaacggcggcgttaccgttacctccatcagcctgaacttcttcaagaagttcgacagctctgctgccgttggcaccaagtacaccgttggcacctccgccttcaacagcctcgtccagaacgtcgccgtcgctgccgacgctttcttcagcaccgtcaagttccacgccgctaccaacggctccatgagcgagcagtacggccgatctgacggcctcatgaccggcgccagagacctgacctggtctcacgctagcctgattagcgcttcttacgccaaggctggcagccccgccgcctaa

SEQ ID NO: 28 - DNA, 1809 bp, synthetic construct
  misc_feature 1..1809: note = .
  source 1..1809: mol_type = other DNA
atgaagctgaactccgtttggaaggctttcagcattctgctcatggctactatggctttcgctgctaccgtccccaagacccaggttaagttagaggcttacacctactctgactctattttctctggccgaattttcgtccagaacatcgactacaccaagattgttaccgtttactggtctgacgcctctaacaagtgggacagctctaagtactacaccgaggccgcttacacccacagcatccccggcaccaactacgagtactgggacttcagcgctaccattggccctagcggcatcaagcagttctacatctcttaccaggtccgaggcgtcacctactacgacaacaacggcggctacggcgtcaactacgacgtcatctccagccctcctaccacctctgacgccaccaccattcctaccaccaccacctccgctattcctagctctaccaccaccatccctgccagcaccggcgtccctagcggcaactccaccattaccgtttgggctgactcccagcacaagatttcttggaaggctatgctcgctaacattaaccctcccggcagcgccaccggcttcattgctgcctccctgtctacctccggccctgactactactacgcttggacccgagacgccgcaatggttgctcacgttatcgttaacgcttacaacaccaccaaggctggcgacgctaccaccctgggcgttctgaaggacttcgttaccttccagatcaaggctatggctacctctaccgtttgcaactgcctgggcgagcctaagttcaaccccgacggctcctcttacaccggcccttggggccgcccccagaacgacggccctgctgagcgcgctaccaccttcatcctgttcgccgactcttacctgtctcagaccggcgacgctgcttacgtcaccaccctgaagcgagccattttcaccgacctcgactacgttgttaccacctggcaggacaactgcttcgacctgtgggaggaggttaacggcctgcacatgtacaccctcgctgttatgcgacgatctctcgttgacggcgcctctttcgccgctcgaaacggcgacaacacccgagcctctacctacaccaacaccgccaagtctattgagaccaagctcgcctctttctacaacagctcctctaactacgtcgttgttaccaagaacttcgctggcggcgttaacaaggctggcctggacacctccaccctgattgctgccaacaccgctggcctgggcgacggcttcttcacccccggcagccccgagatcctcgctaccgccgctgccattgagaagtctttcgccgacctgtacggcattaacaagcagattcctaactggctgggcaccgctattggccgataccccgaggacacctacaacggcaacggcaactctcagggcaacccttggttcatttgcaccgccaccttcgccgagctgtactaccgagctattctggagtggcagcacgccggcagcgttaccgtcaacaacgttaacctgcctttcttcaagaagttcgactctagcacctccagcggcaccacctacaccgttggcaccagctccttcgacagcctgattagcaaggttgcttacgccgctgacaacttcttctctaccattaagtaccacgctgccaccaacggcagcatgtctgagcagttcaaccgagacaccggcttcatgaccggcgccagggacctgacctggtcgcacgctgccttcattaccgccgctaaggctaaggctggcacccccgtttactaa

SEQ ID NO: 29 - DNA, 1821 bp, synthetic construct
  misc_feature 1..1821: note = .
  source 1..1821: mol_type = other DNA
atgcgccctagcctcgtttggaagtctctcctgatggttgtcctgaccgctgcaatgggcacctacgcccagaccgtccctagcgttcctattaagttagaatcttacacctactctgacaacgttttcgctggccgaattttcatccgaaacattgcttacaccaaggttgtcaaggttttctggtctgacgagtccggcaactggaacggcaacggcaactacgttgacgcctcttactctggctccatctccggcaccaactacgagtactgggagttcagcaccaccattggctctgccggcatctcccagtcttacctgcgatacgacgtcagcggctctacctactacgacaacaacggcaacgctaactacgacattgaggaggacagcacccctacccctaccaccaccaccacctctaccaccaccatcaccaccaccaccacctccggcacctctacccctacctctgttcctaccgaggtccctgccggcaacaccaccgtcaccgactgggttaacgaccagttagaaatttcttggcctagcctcctcaagaacgtcaaccctagcggcgctgttaccggcttcattgctgcatctctgtctaccaacgaccccgactacttctactgctggacccgagacgccgccctcgttgcccgcgtcatggtttacatgtacaacaccaccgaggctggcgacacctccctgcgatctaagttacaggactacgttaccttccagattaactccatgaaaaccgccaccgtttgcaactgcctgggcgagcctaagttcaacaaggacggctccggctacagcggcgcttggggccgccctcagaacgacggccccgctgaccgagctattaccctgattctgttcgctgactctttcatcgcccagggcggcgatgtttcttacatcaccaacaccctgaagcctgctatttacaccaacctggactacgttgttaacacctggtctaacgtttgcttcgacctgtgggaggaggttaacggcgtccacatttacaccctgtctgttatgcgaaagggcctcctggaaggcgctgacttcgcttctcgaaacggcgacagcacccgcgccaacacctaccgatctaccgcgtcctccatcaagacccgactggagtccttctggagcagctctaacaactacattaccgtcacccagtcttactctggcggcgttaacaaggctggcctggacgtcagcaccctcctcgccgctaacggcgccagcatgaacgacggcttctacacccccggctctgacaagatgctcgctaccgccgttgccattgagaactccttcgccggcatttacgccgttaaccagaaccgccccgactggaagggcaccgctattggccgataccctgaggacacctacgacggccacggcaactcccagggcaacccttggttcattgctaccgccgcttacgctgagatgtactaccgagctatactggaatggcagcagcagccttccattaccgttaactctattaacctcagcttcttcaagaagttcgactctagcgctgctgttggcaccgtttacaagcccggcacccaggctttcaacaacatggtttctaacgttgctttcgctgccgacgagttcttcagcaccatgaacttccacagcgctaccaacggctctatgagcgagcagtacaaccgaaacaccggcattatgcagggcgcaagagacctgacctggagccacgctgctttcattaccgctgctaaggccaagctgggcacccccgttttctaataa

SEQ ID NO: 30 - DNA, 1809 bp, synthetic construct
  misc_feature 1..1809: note = .
  source 1..1809: mol_type = other DNA
atggttttcagcaagctgttcaagaagcctattctcctgattgttacctacctgaccgttaccgtcgtcgccgagaccgttcctacctctgctcaggtccaggttaagtctttcaactacgacggcagcaccctgtccggccagatttacgtccagaacattgcttacgaaaagaccgttaccgttgtttactctgacggctccgacaactggaacaacaacggcaacaccattgctgcctcttacagctctagcattagcggctctaactacgagtactggaccttcagctctagcgtccccagcattaagcagttctacatcaagtacgttgtcgctggcaagacctactacgacaacaacggcaccaagaactaccaggtttccgcttccacccctaccaccaccaccaccaccacctcctctggcgccaccaagaccaccaccaccatcggccctaccagcaccagcaccgttttccctagcggcaacagcaccatcagctcttggctgaagggccagattgagacctcccgattcgccatgctgcgaaacattaaccccgctggcaccgtcaagggcttcatcgccgcctcgctgtctaccgctaaccccgactacttctacgcttggacccgagacgctgctctcgtcggccacgtcattgctaacgactacaaccgaaccctcgctggcaacagcacctacctgggcctcctgaaggactacgtcaccttctctgttaactcccagtctacctccaccatttgcaactgcctgggcgagcctaagttcaacaaggacggctctggctactctggcgcttggggccgccctcagaacgacggccccgccgagcgagctgacaccttcatcctcattgccgactccatcctgaagcagaccggcgacgccacctacgttaccggcaccctgcgccctgctatttacaaggacctcgactacgtcgttaacgtttggtctaacggctgcttcgacctgtgggaggaggttaacggcgtccacttctacaccctcatggttatgcgacgatctctgatcctgggcgctaacttcgcttctcgaaacggcgacagcacccgagcgtccacctacaccaacaccgctaactctattaagaccaagattgacaccttctggtcttcctctaacaactacgttgctgtttcccagtctgttaccggcggcgttaacaaggctggctacgacgttgctaacctcatcgccgctaacgttggcagcctcgacgacggcttctacacccccggcagcgagcgaatactcgccaccgctgttgctgttgagtctaagttcgcttcgatttacggcgtcaaccagaacctgccttcttggctgggcaactccattggccgataccccgaggacacctacaacggcaacggcaactctcagggcaacccttggttcattgctaccaacacctacgccgagctgtactaccgcgctattaaggagtggaccaacaacggcggcgttaccgtcaccaacgttaacttcaacttcttcaagaagttcgacagctccgcttctgttggcaccaagtacaccgttggcaccagcgctttcaacaccctgacccagaacgttgccctcgctgccgacaacttcttctctaccgtcaaggttcacgctgccaccaacggctccatgtctgagcagttcggccgagactccggcgtcatgaccggcgcccgcgacctgacctggagtcacgcatcactgattaccgccgccctcgccaagaccggcgcccctgttgcctaataa

SEQ ID NO: 31 - DNA, 1833 bp, synthetic construct
  misc_feature 1..1833: note = .
  source 1..1833: mol_type = other DNA
atgaagttctctaacctcctgaagaagcccctgctcctgattgccggcattctcgctgttaccgtcgttgccgagaccgtccctaccaccgaggctgttaaggtcaagtctttcacctacgacggcagcaccctcgctggccagatttacatcaagaacatcgcttacaccaagaccgttaccgttatttactccgacgcctccgacaactggaacaacaacggcaacaccattgctgcttcttactccgctgctattgctggcaccaactacgagtactggaccttcagcgcccccgtgtccggcattaagcagttctacgtcaagtacgttgtttctggcaccacctactacgacaacaacaactctggcaactaccaggtttccgttaccaccaccaccaccaccgcccctaccaccaccacctctggcggctctagcaccaccaccggcggcagcaccaccaccgccacctccgtccctaccggcgtccctagcggcttccctaccggcaacagcaccattagctcttggattgacggccagacctctgtttcccgatacgccatgctgcgaaacattaaccctgctggcgctgttaccggcttcattgccgcatccatgtctacctctggccccgactacttctacgcttggacccgagacagcgccctgacctctcacgtcgttgcttacgactacaacaccaccctcgccggcaacagcaccatcctgggcctcctgaagaactacgttaccttcagcctgaactctcagaccacctctaccgtttgcaactgcctgggcgagcctaagttcaacaaggacggctctggctactccggcgcttggggccgcccccagaacgacggccccgctagtcgagctgacaccttcatcctcatcgctgacagcattctgaagcagaccggcgacgccacctacgtcaccggcaccctcgcccccgctatttacaaggacctcgactacgttgtctctacctggtctaacggctgcttcgacctgtgggaggaggttaacggcgttcacttctacaccctcatggttatgcgacgaggcctcgtcaagggcgctaacttcgcatcccgaaacggcgactctacccgagctaccacctacaccaacaccgccgcgagcattaagaccaagattgactccttctggaactctaacggccagtacgtcagcgtttcccagtctgttaccggcggcgtttccaaggctggctacgacgccagcgtcctgattgcttctaacctgggctctctccaggacggcttctacacccctggcagcgacaagatgctcgccaccgctgtcgccattgagtccaagttcgccagcctgtactccattaaccagaacctgaacggctacctgggcaacgctattggccgataccccgaggacacctacaacggcaacggcaactcccagggcaacccttggttcatttgcaccaacgctttcgccgagctgtactaccgagctattaaggagtggttcaacaacggcggcgttaccgttacctccattagcctgaacttcttcaagaagttcgacagctctgccgccgttggcaccaagtacaccgttggcaccagctctttcaacagcctcgtccagaacgttgctgttgctgccgacgctttcttcagcaccatcaagttccacgccgctaccaacggctccatgagcgagcagtacggccgaaccgacggcctcatgaccggcgctagagacctgacctggagtcacgcatctctgatcagcgcctcttacgctaaggctggctctcccgctgcctaataa

SEQ ID NO: 32 - DNA, 1830 bp, synthetic construct
  misc_feature 1..1830: note = .
  source 1..1830: mol_type = other DNA
atgatgcctaaccacatttggaagtgcctgttcctcgccatgaccaccgttttcatgaccgtcctcgtccagggcgctcccacctctgagattgaactagactcttacacctacaccggcggcgttttctccggccgactgtacgtcaagaacatcgcttacaccaaggaggttaacgtttactggtctgacgccagcgaggactgggctaacaacggcaactacgttgccgctacctactctgaggctatctccggcaccaactacgagtactgggagttcagcgctaagattggctctagcggcatcagcgagtcttacatcaagtacaccgtttccggctctacctactacgacaacaacggcggcaagaactacgccatcaccgagaccaccacccctaccattaccgccacccctacctctaccaccgctaccaccaccattagctctaccaccaccaccgccacctccatccctacctctgttcctagcaccgtccccgagggcaacgttaccgtcaccgagtgggttaacaagcagctcaaaatatcttggtccgacctcttgcaaaacgttaacccttctggcaccgttaccggcttcattgctgcctcactcagcacctccaaccccgactacttctactgctggacccgcgacgccgcaatggttgcccgcgttatgacctacatgtacaacaccaccgaggctggcgacagcagcctggagagcgccctgaaggactacattaccttccagattaactctatgaaaaccgctaccgcttgcaactgcctgggcgagcctaagttcaacaccgacggctccggctactccggcccttggggccgcccccagaacgacggccctgctgagcgcgctaccaccatgattctgttcgctgactcttacctcgcccagggcggcgacacctcttacgttaccaacaccctgaagcctgctatttacaccaacctggactacgttgttggcacctggtctaacaactgcttcgacctgtgggaggaggttaacggcgttcacattttcaccctcgccgtcatgcgaaagtctctcctcgacggcgctgacttcgctgcccgaaacggcgacacctcccgcgttagcggctaccagagcaccgcttctagcattaagaccaagctggagtctttctggtctagctccaacaactacattaccgtcacccagtcttactctggcggcgtccagaaggccggcctggacgtcagcaccctgattgctgctaaccagggctcgatgggcgacggcttctacacccctggctctgacaagatgctcgccaccgctgttgctattgagaactctttcgctaacatttacaccattaacaagaacaagccttcttggctgggcaccgctattggccgataccccgaggacacctacaacggcaacggcaacggccagggcaacccttggttcatcgctaccgccacctacgccgagctgtactaccgagctattttggaatggcagcagcaggagtctgttaccgttaacagcgtcaacttcgacttcttctctaagttcgacagcagcgccaaggttggcaccgtttacacccctggcaccgacaccttcaacactatggtttctaacgttgctttcgctgccgacgagttcctcagcactatggagcactacgctgccaccaacggctctatgtctgagcagttcaaccgagagaccggcagcctgaccggcgcccgagacctgacctggtcgcacgctgctttcattaccgctggcaaggccaagctgggcattcctagcttctaataa

SEQ ID NO: 33 - DNA, 1830 bp, synthetic construct
  misc_feature 1..1830: note = .
  source 1..1830: mol_type = other DNA
atgaagttcagcaagctgttcaccaagcctaccctgttcatcgtttctattattatcagcgttgcttctagcgagaccgtccctaccaccgctaacgtcctcgttaagtcttacacctgggacggcgctaccctgtctggccagatttacattaagaacctcgcttacgccaaggttgttagcgttatttactctgacgctaacgacaactggaacaacaacggcaacaaggttgccgcttcttactctgctggcattgacggcaccaactacgagtactggaccttcagcggcgccgtttccggcatcaagcagttctacgtcaagtacgacgtcagcggcacctcttactacgacaacaacggcaccaagaactaccaggttaccaagacctctaccaccaccaccgccgctaccaccaccaccaccgctaccaccaccaccggcggcaccggcaccaccaccaccaccaccgccaccgccaccgctaccagcaccgacttccctaccggcaacagcaccattaccacctggctgaagtcccaggaggacatctcccgaggcgccatgctccgaaacattaacccccctggcgctgctaccggcttcattgctgccagcctgtctacctccggccccgactactactacgcttggacccgagactccgccctgacctctcacgtcatcgctcacgactacaacaccaccctcgctggcaacaccaccatcctgaacatcctgaaggactacgttaccttctccgtcaagtcccagtccgtttctaccgtttgcaactgcctgggcgagcctaagttcaaccccgacggcagctcttacaccggcgcttggggccgcccccagaacgacggccctgccgagcgagcctccaccttcatcctgttcggcgactcttacctgaagcagaccggcgacgctacctacgttaccggcaccctcgctcctgccatttacaaggacctcgactacgttgttaacacctggtctaacggctgcttcgacctgtgggaggaggttaacggcgttcacttctacaccctcatgtctatgcgacgaggcctcctcgacggcgccaacttcgccaagcgaaacggcgacaccacccgagctaccacctacaccaacaccgccgcttccattgctaccaagattgacaccttctgggttagctctggcaactacgtccaggtttcccagtctgttaccggcggcgtcagcaaggctggctacgactgcgctaacattctcgctgctaacatggctgctaaccacgacggcttctacacccctggcagctctaagattctcgctaccgccgttgccattgagtctaagttcgccagcctgtactctattaactctggcctcgcttcttggctgggcaccgccattggccgataccccgaggacacctacaacggcaacggcaactctcagggcaacccttggttcatttgcaccaacgctttcgctgagctgtactaccgagccatcaaggagtggacccaggctggctccgttaccgttgacagcacctctctgaacttcttcaagaagttcgacagctctgctgccgccggcaccaagtacaccgttggcacctctgctttcaccaacctcgtccagaacattgctaacggcgctgacaagttcctgtctacctccaagttccacgccgctaccaacggctccatgtctgagcagtacaaccgagacagcggcctcatgaccggcgcccgagacctgacctggtcgcacgcctcactgattagcgcctcccgagctaaggctggctctcctagcctctaa

SEQ ID NO: 34 - DNA, 1845 bp, synthetic construct
  misc_feature 1..1845: note = .
  source 1..1845: mol_type = other DNA
atgaagcctattcacctgtggcactacctgttcctcgccatgaccgctgccttcatgggcgtcctcgctgacgtccctacctccgccaacattgaactcgactactacacctacaaggacaaggttttctccggccgaatttacgtcaagaacatcgcttacgaaaaaaccgttgctgtttactggtctgacgctagtggcgactggaacaacaacggcaacaacgttgccgcttcttactctgagtccatctccggcaccgactacgagtactgggacttcagcaccaccattggctctggcggcatcaagcagtcttacctcaagtacaccgttagcggcaacacctactacgacaacaacggctctaacaactacgacattaccgagaccaccgctaccaccaccaccaccaccaccaccaccaccaccaccggctctaccgccaccacctctaccacctctggccctaccagcaccacctctaccacccccggccctacccctaccaacgttcccgacggcaacgttaccgtcaccgagtgggttaacacccagttcgctatttcttggcctaccctcctgaagaacgtcaaccctagcggcaccgtcaagggcttcattgctgcatcactcagcaccaacaaccctgactacttctactcttggacccgagacagcgctctcgttgctcacaccatgacctacctgtacaacacctctgaggctggcgacagcaccattgagtctgccctgaaggactacgttaccttcagcattaacgctatgaacgctgctaccgtttgcaactgcctgggcgagcctaagttcaacaccgacggctccggctacaccggcgcttggggccgcccccagaacgacggccccgcaagtcgagctaccaccatgattctgttcgccgactccttcctcgcccagggcggcgatgtttcttacgttattaacaccctcaagcctgctatttacaaggacctggactacgtcgttagcacctggtctaacacctgctacgacctgtgggaggaggttaacggcgttcacatttacaccctgtctgttatgcgccgcgccctgattgacggcgctaacttcgcccagcgaaacggcgacacctcccgcgtcagcggctacaccagcaccgccaccaccatcaagacccgactggagtctttctggagcgactctaacaactacattaccgttacccagtcttactccggcggcgtccagaaggccggcctggacgtcagcaccctgattgctgccaacattggcagcgttggcgacggcttctacacccccggctctgacaaggtcctcgccaccgccgtcgccatcgagaagtccttcgctaacctgtacaccattaaccagaacaagccttcttggctgggcaacgccattggccgataccccgaggacacctacgacggctacggcaactccaagggcaacccttggttcattgctaccgctacctacgctgagctgtactaccgagctatcctagagtggcagcagcaggcttccgttaccgttaactctattaacctgggcttcttctccaagttcgacagctctgcctctgttggcaccgtttacacccccggcaccgactctttcgccaacatggtttctaacgttgctttcgctgctgacgagttcctgtctaccatcgactaccacgccatgaacaacggctctatgcacgagcagtacaaccgagacaccggcatctcccagggcgcacgcgacctgacctggtcacacgctgctttcattaccgctgccaaggctaagctgggcgctcccgctttctaataa

SEQ ID NO: 35 - DNA, 1818 bp, synthetic construct
  misc_feature 1..1818: note = .
  source 1..1818: mol_type = other DNA
atgaagctcatgaactctagcatgaagacctgcgttttcttcatcctgtcttacttcagcctcctcgtcagcagcgctgccgttcctacctctgctgctgttcaggttgagtcctacaagtacgacggcaccaccttcagcggccgaattttcgtcaagaacattgcttactccaaggttgttaccgtcatatatagcgacggctctgacaactggaacaacaacaacaacaagatcagcgctgcttacagcgaggctattagcggctccaactacgagtactggaccttctctgctaagctgtccggcattaagcagttctacgtcaagtacgaggtttctggctctacctactacgacaacaacggcaccaagaactaccaggtccaggctacctctgccaccagcaccaccgccaccgccaccaccaccacctctaccagcaccaccaccaccagcaccggccctaccagcaccgcaagtgtttctttccctaccggcaactccaccattagctcttggattaagaaccaagaagaaatttcccgattcgccatgctgcgaaacattaacccccccggctccgccaccggcttcattgctgcctctctgagcaccgcgggacccgactactactactcttggacccgagacagcgccctgaccgctaacgttattgcttacgagtacaacaccaccttcgctggcaacaccaccctcctgaagtacctgaaggactacgttaccttctccgtcaagtctcagtctgttagcaccgtttgcaactgcctgggcgagcctaagttcaacgccgacggcagctcttacaccggcccttggggccgaccccagaacgacggccccgccgaacgggccgtcaccttcatgctcattgctgactcttacctgacccagaccaaggacgcttcttacgtcaccggcaccctgaagcccgccattttcaaggacctcgactacgttgtttccgtttggtctaacggctgctacgacctgtgggaagaagttaacggcgttcacttctacaccctgatggttatgcgaaagggcctcatcctgggcgctgacttcgctgctcgaaacggcgactctagccgagcctctacctacaagaacaccgcgagcaccatggagtccaagatctctagcttctggtccgactctaacaactacgtccaggtttcccagtctgttaccgctggcgtctccaagaagggcctcgacgttagcaccctcctcgccgctaacattggctccctgcccgacggcttcttcacccccggcagcgagaagattctggctaccgccgttgccctagaaaacgctttcgcatctctgtaccctattaactctaacctgccttcttacctgggcaactccattggccgataccccgaggacacctacaacggcaacggcaactcccagggcaacccttggttcctcgccgttaacgcttacgctgagctgtactaccgagctattaaagagtggatctctaacggcaaggttaccgtctctaacatcagcctccctttcttcaagaagttcgacagctccgccacctctggcaagacctacaccgctggcaccagcgacttcaacaacctcgctcagaacattgccctgggcgctgaccgattcctgtctaccgttaagttccacgcttacaacaacggcagcctgtctgaagagtacgaccgatctaccggcatgtctaccggcgcacgcgacctgacctggtctcacgcctcgctcatcaccgccgcttacgccaaggctggcagccccgctgcctaa

SEQ ID NO: 36 - DNA, 1413 bp, synthetic construct
  misc_feature 1..1413: note = .
  source 1..1413: mol_type = other DNA
atgaagtctccttacagcatgaaaaccgtcctcgctctcctgtccctgacccctctgttcaccagcgacgttgttgccgttcctagcggcaactccaccatcaccgcttggggctccaagcaggacggcatctctttcagcaccatgctgggcaacatcaaccctcccggcagctctaagggcttcattgctgcctcgctgtctaccgctggccctaactactactactcttggacccgagacagcgctctcgtcgcccgcgccattacctacaagtactccacctcttaccagaacgaccccaagattctgggcctcctgaaggactacgttacctaccaggttaacgagcagaccgagtctaccgtttgcaactgcctcggcgagcctaagttcaaccccgacggctctagcttcagcggcccttggggccgccctcagaacgacggccctgctgagcgcgcttccaccatgattctgttcgccaagtcttactacgcccagaccaacgacgttggctacgtttctaacaccctgaagcccgctatttacaaggacctggactacatcgttaacgtttggggcaacaactgcttcgacctgtgggaggaggttaacggcgtccacttctacaccctcatgatgatgcgacacggcctcgtccagggcagcattttcgctaacaccctcggcgacagcacccgagctaacacctacaagaccgccgctcagaacattaagaaccgaattgacaccttctgggagtctggctctaactacattgttgttacccagaaccagtctgctggcgttaacaagccttccggcctcgacgttgccgtcctgctggctgccaaccagggcggcctgggcgacggcgtttacacccccggctctgacaaggtcctcgccaccgccgtcgctctggagaagtctttcgcaagcctgtaccccattaacaagaacctgccttcttactacggcaccgccattggccgataccccgaggacacctacaacggcaacggcaacagcgaggctaacccttggttcattgccaccaccacctacgctgagctgtactaccgagctatttccgagtggaccaacggctccggcgtcaccgtcaactctattaacaaggagttcttctctaagttcgacgcctctgccaccaacggcaaggtttacacccccggcagcgactctttcaactccctggtcaacaacgttgctattgctgctgacaacttcctcagcaccgtccgataccaccagacctccaacggcagcctgtctgagcagttcaaccgatacaccggcttcatgaccggcgcacgggacctgacctggtcccacgccgctatggttaccgctctcgccgctaaggctggcacccctagcccttaataa

SEQ ID NO: 37 - DNA, 1830 bp, synthetic construct
  misc_feature 1..1830: note = .
  source 1..1830: mol_type = other DNA
atgcagctgaacctgtactggaagagcctcgccgtcctcgctctgattcagatggttatggctgctaccgtccctaccacccaggtccagctcgactactacacctactctaacaacgtcctgtctggccgaatctacgttcagaacattgcttacagcaaggttgttaaggttatctatagcgacgcctccggcaactggaacaacaacggcaacaccatctccgcttcttacgtcgagtctatttccggcaccaactacgagtactgggacttcagcgccaccattggcaccgctggcatcaagcagttctacctgcgatacgacgtttccggctccacctactacgacaacaacggcggcgacaacaacaactacaacgttgttgctaccacctctaccacctctagctctaccaccaccaccaccgctaccaccaccaccgccaccaagaccaccagcaccacctctgccaccgctacccctacctctagcgccaccttccctagcggcaactctaccattaccacctgggccaagtctcagaaggacatttcttggaagaccctcctgaccagcctgaaccctagcggcaccgccaagggcttcattgctgcgagtctgtctacctctaaccctgactactactacgcttggacccgagacagcgctctcgttgcccgaaccatggttaacatgtacaacaccaccgaggctggctctgcttcagtcctgggcctcctgcaggactacgttaccttccagattaacgccatgagcaccagcaccgtttgcaactgcctgggcgagcctaagttcaaccccgacggcagctcttacaccggcgcttggggccgcccccagaacgacggccccgctgaacgtgcaagcaccttcatcctgttcgctgactcttacattgcccagggcggccagctgtcttacgtcaccggcaccctcgctcccgccatctacaaggacctgaactacgtcgtttctacctggtctaacaactgcttcgacctgtgggaagaggttaacggccgacacatgttcaccctcgccgtcatgcgacgcgccctcctggacggcgttaacttcgcctcccgaattggcgacaccacctactctagcacctggagctctaccgcttctagcatccagtctaccctgtctggctactacctgtcttccggcaactacattaccaccgttcagaacttccagtctggcgtttccaaggctggctacgacgtcagcaccctcatcgctgctaacgttgcgggcatgggcgacggcttcttcacccccggctctgacgaggttctcgctaccgccgttgctattgagaacaagttctctagcctgtacggcgtcaacgctaacaaggcttcttacctcggcaccgccattggccgataccccgaggacacctacaacggctacggcaacggccagggcaacccttggttcattgctaccgctgcttacgccgagctgtactaccgagctattctggagtggcagaccaagagcagcatcgttgttaactccaagaacctcggcttcttctccaagttcgactcttctgccgccgttggcaccacctacacccccggcaccaccgcttactctaacatggtccagaacgtcgccctcgctgctgaccgattcctcagcaccgtccagctgcacgctgccaccaacggctccatgtctgagcagttcaaccgagacaccggcgttatgcagggagcccgtgacctgacctggtctcacagcgctttcattaccgctgcccgagctaagctgggcagccccgtttactaa

SEQ ID NO: 38 - DNA, 1404 bp, synthetic construct
  misc_feature 1..1404: note = .
  source 1..1404: mol_type = other DNA
atgcgagctaccaccgctattctgtctctgttcaccaccgcctctatggctctggctgccaactctaccgtccctgacggcaactccaccaccaccgcttgggtcaagaagcaagaagccatctcttggaccgacctgaagaccaacgtcaaccctgagggcgctgctaagggcttcattgctgcctctctgtctaccagcgagcctgactactactacgcttggacccgagactctgccctcgttgctcgcgttatggttaacaagtacaacaccaccgacgctggggacgccaacctcctcggcctcctgcaggactacgtttctttccagatcaacgcaatgggcgagtctaccgtttgcaactgcctgggcgagcctaagttcaaccccgacggcagctcttacaccggcgcttggggccgcccccagaacgacggccccgccgagcgagcttctaccttcattctcctcgccgacagcatgattgcccagaagtctgctaacggctcttacgtcagcgacaccctggctcccgctatctacaaggacctggcttacgttgcaagcacctgggagaacgcttgctacgacctgtgggaagaggttaacggcaagcacatgtacaccctcagcgtcatgcgacgagccctcctggacggcgccgacttcgcttctcggcagggccagaccgccaacgttacctcttggaagtctaccgctgacaagattaagtctagcctggagggcttcttcagctccgacaacggctacattgaggttacccaggacatgcagggcggcgtccagaagaagggcctggacgtttccaccctcatcgctgctaacattggcagcatgggcgacggcttctacacccccggcagcgacgaggttctcgccaccgctgttgccgttgaggctgccttcgctgacctgtacaagattaaccagaacaccaactccaccggcctcggcaccgccatcggccgataccccgaggacacctacaacggcgttggcaactcccagggcaacccttggttcattgctaccaacaccttcgctgagctgtactaccgcgccatcctggagtggcagaacaagggcaccattaccgttaactccgttaacgctgctttcttcagcaagtttgactctagcgctaaggccggtacaacctacaagtctggctctaccgagtttgatagcctcattaacaaggttgctctggctgctgacgctttcctgaacaccgtccagacctacgctgcttctaacggctctatgtctgagcagtacaaccgagacaccggcgccctgaccggcgcccgagacctgacctggtctcacgcaagtctcattaccgccgctaacgccaagctcggcaccccttacaactaa

SEQ ID NO: 39 - DNA, 1419 bp, synthetic construct
  misc_feature 1..1419: note = .
  source 1..1419: mol_type = other DNA
atgaagctcggccgaattggcttctctattctgtctgttgcctccgttttctctcaggccgtcgttaccgctgccgctgccgcctctgtccctagcggcaacgccaccattacctcttggattcaggagcagctcgctatttcttggaacaccatgctgaccagcgttaaccccgttggcgccgtcaccggcttcatcgctgcgagtctgtctaccgctaaccccgactactactactgctggacccgagacgctgctctcgtcgcgagagttatgaccttcatgtacaacaccacccaggctggcgacagcggcctcctgggcatcctccaggactacgtttccttccagatccacgcaatgggcgagtctaccgtttgcaactgcctgggcgagcccaagttcaaccccgacggcagctcttacaccggcgcttggggccgccctcagaacgacggccctgctgagcgagcctctaccttcatcaagattgctgactcttacctgacccagaccggcgatgtatcctacgttaccaacaccctcaagcccgctatttacgaggacctggactacattgttaacgtttggcagaacacctgcttcgacctgtgggaggaggtttacggaatgcacatgtacaccctggctgttatgcgacgaggcctcctcgacggcgccgacttcgctacccgaaacggcgacaccgacaaggcctctacctacaccagcaccgccaccagcatccagacccgactggccaccttctggtctgactccaacggctacattaccgttacccaggactaccagtccggcgtcagcaaggctggcctggacgttagcaccctgattgctgctaacgttgccggcatgaacgacggcttcttcacccctggctctgacgagattctcgctaccgccgtcaaggttgaggctgctttcgctaacctgtacggcattaacattaacaaggcctcttacctgggcaacgccattggccgataccccgaggacacctacaacggcaacggcaactctgagggcaacccttggttcattgccaccgctgctttcgctgagctgtactaccgcgccattatggagtggcagctccgaggcagctctattaccgtcaactctgttaaccagggcttcttcaccaagttcgaccctagcgccaccgccggcaccacctacacccccggcaccgacgctttcaactccctcattgacaacgttgccctcgctgctgaccagttcttcagcaccatccacctccaccgcgccaccaacggctctatgtctgagcagtacaaccgagacaccggcttcatgcagggcgctcgggacctgacctggtcccacgccgctttcatcaccgccgctaaggctaagcagggcacccctagcttctaa

SEQ ID NO: 40 - DNA, 1842 bp, synthetic construct
  misc_feature 1..1842: note = .
  source 1..1842: mol_type = other DNA
atgcgccccgttcacctgtggaagagcctgctcctcgccgctgctaccgctgttaccggcaccatcgccgttgatgttccttctgttcctatccagctagagtcttacacctactctgagaacgttttcgccggccgaattttcgtccagaacattgcttacaccaaggaggttaacgttttctggtctgacgcttccgacgactggaacgacaacggcaactccgttgctgcttcttactccgagtctattgctgacaccaactacgagtactgggagttcagcaccaccattggctctgccggcatctcccagtcttacctgcgatacgatgtgtctggctctatttactacgacaacaacgactcccagaactacgacattaccgagaccagcacccctaccaccaccaccgctgcccctacctctaccacctctacccctaccaccaccaccgacggcggcagcaccaccaccaccaccgccacctccgtccctacctctaccggcgtccctgagggcaacgctaccattagcgagtgggcatctgcccagctggacatctcttggcctaacctcatgatgaacgttaaccctagcggcgctgttaccggctccattgttgcctcactgtctacctctaaccctgactacttctacatttggacccgagacgctgcgatggttgcccgcgttatggtttacatgtacaacaccaccgaggctggcgacaccaacctgctcaacgccctgaccgactacgttaccttctctatttccagcatgaacgttgacaccgtttgcgactgcctgggcgagcctaagttcaacgttgacggctctggctacaccggcgcttggggccgcccccagaacgacggccccgccgagcgcgcctctaccatgatcctgattgctgactcttacatcgcccagggcggcgatgtatcttacgtcaccgacaccctcaagcctgctatttacaccgacctggactacgtcgttgacacctggtctaacgtttgcttcgacctgtgggaggaggttaacggcattcacatgtacaccctcagcgtcatgcgaaaggccctcctggacggcgccaacttcgctacccgaaacggcgacacctcccgcgttagcggctacgagagcaccgcttcatccatcaagacccgactggagtctttctggtctagctctaacaactacattaccgtcacccagtccttcagcggcggcgtttccaaggccggcctggacgtcagcaccctgattgctgccaacctcgcttccatgaacgacggcttctacacccccggctctgacgagattctcgctaccgccgttgctattgagaactctttcatctccgagtacaccctcaaccagaaccgcccttcttggctgtccaccgccattggccgataccctgaggactcttacgacggctacggcaactcccagggcaacccttggttcatcgctaccgctacctacgctgagctgtactaccgagctatcctagagtggcagcagcaggcttcaattagcgttaacagcgttaacctgggcttcttcagcaagttcgactctgacgcttctgttggcaccgtttacacccccggcaccgaggacttcgccaacatggtttctaacgtcgctttcgctgctgacgagttcctcgccaccattgagaaccacagcgccgtcaacggcagcctcagcgagcagtacaaccgagacaccggcattatgcagggcgcaagagacctgacctggtcacacgctgccttcattaccgctgccaaggccaagcagggcgctcctattcactaataa

SEQ ID NO: 41 - protein, 19 aa, Saksenaea vasiformis
MPSWKTLFLLLGPIATAAA

SEQ ID NO: 42 - protein, 23 aa, Backusella circina
MKLFGTLKASFVFLIGCIVFAMA

SEQ ID NO: 43 - protein, 24 aa, Backusella circina
MKPFGPIKTTLFIIISHFSLNSLA

SEQ ID NO: 44 - protein, 24 aa, Benjaminiella poitrasii
MKFSHLFRKPTLLIVTFLTATVFA

SEQ ID NO: 45 - protein, 24 aa, Choanephora cucurbitarum
MVSFNFLKKPVFIVITCLTVSVCA

SEQ ID NO: 46 - protein, 25 aa, Rhizopus stolonifer
MKLFGLPLKPSLFFVVSYLSFLASA

SEQ ID NO: 47 - protein, 24 aa, Mucor circinelloides
MKFSNLLKKPLLLIAGILAATVVA

SEQ ID NO: 48 - protein, 21 aa, Dichotomocladium elegans
MKLNSVWKAFSILLMATMAFA

SEQ ID NO: 49 - protein, 23 aa, Fennellomyces sp.
MRPSLVWKSLLMVVLTAAMGTYA

SEQ ID NO: 50 - protein, 24 aa, Gilbertella persicaria
MVFSKLFKKPILLIVTYLTVTVVA

SEQ ID NO: 51 - protein, 24 aa, Mucor circinelloides
MKFSNLLKKPLLLIAGILAVTVVA

SEQ ID NO: 52 - protein, 25 aa, Circinella umbellata
MMPNHIWKCLFLAMTTVFMTVLVQG

SEQ ID NO: 53 - protein, 24 aa, Mucor cordense
MKFSKLFTKPTLFIVSIIISVASS

SEQ ID NO: 54 - protein, 23 aa, Phascolomyces articulosus
MKPIHLWHYLFLAMTAAFMGVLA

SEQ ID NO: 55 - protein, 27 aa, Rhizopus microsporus
MKLMNSSMKTCVFFILSYFSLLVSSAA

SEQ ID NO: 56 - protein, 26 aa, Spinellus fusiger
MKSPYSMKTVLALLSLTPLFTSDVVA

SEQ ID NO: 57 - protein, 21 aa, Syncephalastrum racemosum
MQLNLYWKSLAVLALIQMVMA

SEQ ID NO: 58 - protein, 19 aa, Syncephalastrum racemosum
MRATTAILSLFTTASMALA

SEQ ID NO: 59 - protein, 20 aa, Thermomucor sp.
MKLGRIGFSILSVASVFSQA

SEQ ID NO: 60 - protein, 23 aa, Zychaea mexicana
MRPVHLWKSLLLAAATAVTGTIA

SEQ ID NO: 61 - protein, 449 aa, Saksenaea vasiformis
APVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSESTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 62 - protein, 582 aa, Backusella circina
DTVPTTQVKLQSYTYSGGVLSGTIYVQNIDYTKVVTVIYSDGSGNWNNNGNTISASYSSSISGTNYEYWTFSASVSSIQQFYISYLVDGTTYYDNNGGYGANYAVTATTTTASSTTKTTSGTTKTSTTSTATATSTGTSSFPSGNSTISTWSPSQHSISLYAMLRNINPPGSAAGFISASLSTSGPDYYYSWTRDSALVAHVIVNEYNTTYQGNSTLLGILKDYVTYSLNAQTTSTVCNCLGEPKFNPDGSSFTGAWGRPQNDGPAERAVSFIYFADSYLTQTSDSSYVTGTLAPAIYKDLDYVVSVWSNGCFDLWEEVNGIHFYTLMFMRRGLLDGANFASRNGDSTRASTYTSTAASIKTKIDGFWVSSGNYVQVSQSVTSGVSKAGYDASTLIAANQASRGDGFYTPGSDKMLATAVAIESQFSSLYSINTNKASYLGNAIGRYPEDTYNGNGNSQGNPWFLCTNAFGELFYRAISEWNAAGSVTVNSVNLAFFKKYDSSTSSGTTYTVGTSAYNNLVQNVALAADAYFSTVKYHALTNGSMSEQYDRSSGMATGARDLTWSHAAMITALKAKSGTPVY

SEQ ID NO: 63 - protein, 578 aa, Backusella circina
DTVPTTQVEVKTYTYSGGELSGTIYVENIDYTKVVTVIYADGSDDWNNNGNTIAAAFSESISDTNYEYWTFSSSVNSIKEFYVKYDVDGNTYYDNNGNANYQVSASSTTTTTATKTTTTATKTTATATSTATGSTSFPSGNSTISSWIKGQEDTSRSVMLGNINPPGSATGFISASLSTSGPDYYYHWTRDAALVAHVIVNDYNTTLSGDSSTLQVIKDYVTFSVNSQSESTVCNCLGEPKFNPDGSSYTGAWGRPQNDGPAERATTFIFFADTYLAQGGDSSYVTGTLAPAIYADLDYVVNNWSTGCYDLWEEVNGIHFYTLMVMRRGLIDGASFASRNGDSTRSSSYTSTAKSIATKIDSFWSASNNYVAVSQSVTSGVSKAGYDASTIIAANQASLGDGFYTPGSDKMLATSVAVENAFSSLYTVNTGKASYLGNAIGRYPEDTYNGNGNSQGNPWYLCTNALGELYYRAIKEWNAAGSVTVNSVNLGFFQKIDSSISSGTTFTLGTSDYSTLVDNVALAADKFFAVVQYHERSNGSMPEQFGREDGLPTGARDLTWSHAAMISAARAKAGTPVY

SEQ ID NO: 64 - protein, 577 aa, Benjaminiella poitrasii
ETVPTTSQVKLKSYTYDGSTLSGQIYIQNIAYTKVVTVIYSDASSNWNNNGNTIAASYTNGISGTNYEYWNFTGPVSSINAFYIKYDVSGNTYYDNNNNANYQVTKTSTTTTTSTATATTTTKTSTPTSTGVPSNFPTGNSTISTWLKSQIGISRYAMLRNINPAGSAVGFIAASLSTANPDYYYAWTRDSALTSYVIANDYNSTLAGNSTILQIMKDYVTFSVKSQSVSTACNCLGEPKFNPDGSSYTGAWGRPQNDGPAERATTFILFADSYLKQTGDASYVTGTLKPAIFKDLDYVVNTWTNGCFDLWEEVNGVHFYTLMVMRKGLIRGANFATRNGDSTRASTYTNTAASIKTKMDSFWSSGNNYIAVSQSVTGGVSKAGYDVANIIAANVGSLQDGVYTPGSDRILATAVAVEAKFASLYGVNSNLPGYLGNSIGRYPEDTYNGNGNSQGNPWFLATNAYAELYYRAIREWYDNGGVTVNSVNLPFFKKFDSSAASGTTYTVGTTAFNTMVSNVAAAADKFFSTVKFHAYTNGSMSEQFGRNDGLCTGARDLTWSHASLISAALAKAGTPSV

SEQ ID NO: 65 - protein, 574 aa, Choanephora cucurbitarum
QSVPTSDPVKVKTFSYDGNSFSGQIYIKNIAYEKTVTVIYSDGTGNWNNNNNKIAAAFSEAISGSNYEYWTFSASVPSIKQFYVKYDVSGKTYYDNNGSKDYNVVTSGPTTTSSGPSKTTTNGPAPTSTNTNFPSGNPSITSWIDKQIDISRSAMLKNINPAGTVKGFIAASLSTSNPDYFYAWTRDAALVAHVVANDYNRTKSGDATYLGLLKDYVTFSINSQNTPTACNCLGEPKFNKDGSGYNGPWGRPQNDGPAERADTFVLIADSILTQTKDVSYVTGTLRPAIYTDLDYVVRTWSNGCFDLWEEVNGVHFYTLMVMRRALLVGANFASRNGDSARASNYNNAANSIKSKIDSFWSSNNNYVAVSQSVTGGVSKAGYDVSTLIAANVGSLSDGFYTPGSERMLATAVAIEDKFANLYGINRNLDSSLGNAIGRYPEDTYNGNGNSQGNPWFIATNAYAELYYRAIKEWNNNGGVTVTNVNLNFFKKFDGSASVGTKYTAGSAAYNTLTQNIALAADKFFNTVKVHAATNGSMSEQYHRDTGSMTGARDLTWSHASLITAALAKAGTPVA

SEQ ID NO: 66 - protein, 584 aa, Rhizopus stolonifer
ASIPSSSEVQLDTYAYDGAKFSGKIYVKNIAYEKTVTVVYADGSDNWNNNGNIISATFSTPISGSNYEYWTFSSSISGIKEFYIKYVVSGKTYYDNNGTKNYQVSTTSSTTTASTTTATRTTTAGTTSTSTTAAPTSTSSGSFPSGNSTVSSWIKRQEKISRFAMLRNINPPGSAAGFIAASLSTSGPDYYYSWTRDSALTSNLIAYEYNTTLSGNTTILNILKDYVKFSISSQTTSTVCNCLGEPKFNHDGSSYTGAWGRPQNDGPAERANTFILFADSYLDQTKDASYVTGTLKPAIFKDLDYVVNVWSNGCYDLWEEVNGVHFYTLMVMRKGLLLGADFAKRNGDSTRASTYTNTASTIATKISSFWVSSSNWIQVSQSVTAGVSKKGLDVSTLLAANLGSVEDGFFTPGSEKILATAVAIEDAFASLYPINSNLPSYLGNSIGRYPEDTYNGNGNSQGNPWFLAVNGFAELYYRAIKEWVNNGSVTVSNISLSFFKKFDSSATAGKTYTAGTADFNNLAQNIALGADRFLSTVQTHAFNNGSLAEEYDRTTGVSTGARDLTWSHASLITAAYAKAGSPAA

SEQ ID NO: 67 - protein, 584 aa, Mucor circinelloides
ETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSSISSWIDGQTSVSRYAMLRNINPAGAVSGFIAASMSTSGPDYFYAWTRDSALTSHVVAYDYNTTLAGNSTILGLLKNYVTFSINSQTTSTVCNCLGEPKFNKDGSSYTGAWGRPQNDGPASRADTFILIADSILKQTGDATYVTGTLAPAIYKDLDYVVSTWSNGCFDLWEEVNGVHFYTLMVMRRGLIKGANFASRNGDNTRANTYTNTAASIKTKIDSFWNSNGNYVTVSQSVTGGVSKAGYDASVLIAANLGSVQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSAFNSLVQNVAVAADAFFSTVKFHAATNGSMSEQYGRSDGLMTGARDLTWSHASLISASYAKAGSPAA

SEQ ID NO: 68 - protein, 581 aa, Dichotomocladium elegans
ATVPKTQVKLEAYTYSDSIFSGRIFVQNIDYTKIVTVYWSDASNKWDSSKYYTEAAYTHSIPGTNYEYWDFSATIGPSGIKQFYISYQVRGVTYYDNNGGYGVNYDVISSPPTTSDATTIPTTTTSAIPSSTTTIPASTGVPSGNSTITVWADSQHKISWKAMLANINPPGSATGFIAASLSTSGPDYYYAWTRDAAMVAHVIVNAYNTTKAGDATTLGVLKDFVTFQIKAMATSTVCNCLGEPKFNPDGSSYTGPWGRPQNDGPAERATTFILFADSYLSQTGDAAYVTTLKRAIFTDLDYVVTTWQDNCFDLWEEVNGLHMYTLAVMRRSLVDGASFAARNGDNTRASTYTNTAKSIETKLASFYNSSSNYVVVTKNFAGGVNKAGLDTSTLIAANTAGLGDGFFTPGSPEILATAAAIEKSFADLYGINKQIPNWLGTAIGRYPEDTYNGNGNSQGNPWFICTATFAELYYRAILEWQHAGSVTVNNVNLPFFKKFDSSTSSGTTYTVGTSSFDSLISKVAYAADNFFSTIKYHAATNGSMSEQFNRDTGFMTGARDLTWSHAAFITAAKAKAGTPVY

SEQ ID NO: 69 - protein, 582 aa, Fennellomyces sp.
QTVPSVPIKLESYTYSDNVFAGRIFIRNIAYTKVVKVFWSDESGNWNGNGNYVDASYSGSISGTNYEYWEFSTTIGSAGISQSYLRYDVSGSTYYDNNGNANYDIEEDSTPTPTTTTTSTTTITTTTTSGTSTPTSVPTEVPAGNTTVTDWVNDQLEISWPSLLKNVNPSGAVTGFIAASLSTNDPDYFYCWTRDAALVARVMVYMYNTTEAGDTSLRSKLQDYVTFQINSMKTATVCNCLGEPKFNKDGSGYSGAWGRPQNDGPADRAITLILFADSFIAQGGDVSYITNTLKPAIYTNLDYVVNTWSNVCFDLWEEVNGVHIYTLSVMRKGLLEGADFASRNGDSTRANTYRSTASSIKTRLESFWSSSNNYITVTQSYSGGVNKAGLDVSTLLAANGASMNDGFYTPGSDKMLATAVAIENSFAGIYAVNQNRPDWKGTAIGRYPEDTYDGHGNSQGNPWFIATAAYAEMYYRAILEWQQQPSITVNSINLSFFKKFDSSAAVGTVYKPGTQAFNNMVSNVAFAADEFFSTMNFHSATNGSMSEQYNRNTGIMQGARDLTWSHAAFITAAKAKLGTPVF

SEQ ID NO: 70 - protein, 577 aa, Gilbertella persicaria
ETVPTSAQVQVKSFNYDGSTLSGQIYVQNIAYEKTVTVVYSDGSDNWNNNGNTIAASYSSSISGSNYEYWTFSSSVPSIKQFYIKYVVAGKTYYDNNGTKNYQVSASTPTTTTTTTSSGATKTTTTIGPTSTSTVFPSGNSTISSWLKGQIETSRFAMLRNINPAGTVKGFIAASLSTANPDYFYAWTRDAALVGHVIANDYNRTLAGNSTYLGLLKDYVTFSVNSQSTSTICNCLGEPKFNKDGSGYSGAWGRPQNDGPAERADTFILIADSILKQTGDATYVTGTLRPAIYKDLDYVVNVWSNGCFDLWEEVNGVHFYTLMVMRRSLILGANFASRNGDSTRASTYTNTANSIKTKIDTFWSSSNNYVAVSQSVTGGVNKAGYDVANLIAANVGSLDDGFYTPGSERILATAVAVESKFASIYGVNQNLPSWLGNSIGRYPEDTYNGNGNSQGNPWFIATNTYAELYYRAIKEWTNNGGVTVTNVNFNFFKKFDSSASVGTKYTVGTSAFNTLTQNVALAADNFFSTVKVHAATNGSMSEQFGRDSGVMTGARDLTWSHASLITAALAKTGAPVA

SEQ ID NO: 71 - protein, 585 aa, Mucor circinelloides
ETVPTTEAVKVKSFTYDGSTLAGQIYIKNIAYTKTVTVIYSDASDNWNNNGNTIAASYSAAIAGTNYEYWTFSAPVSGIKQFYVKYVVSGTTYYDNNNSGNYQVSVTTTTTTAPTTTTSGGSSTTTGGSTTTATSVPTGVPSGFPTGNSTISSWIDGQTSVSRYAMLRNINPAGAVTGFIAASMSTSGPDYFYAWTRDSALTSHVVAYDYNTTLAGNSTILGLLKNYVTFSLNSQTTSTVCNCLGEPKFNKDGSGYSGAWGRPQNDGPASRADTFILIADSILKQTGDATYVTGTLAPAIYKDLDYVVSTWSNGCFDLWEEVNGVHFYTLMVMRRGLVKGANFASRNGDSTRATTYTNTAASIKTKIDSFWNSNGQYVSVSQSVTGGVSKAGYDASVLIASNLGSLQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSSFNSLVQNVAVAADAFFSTIKFHAATNGSMSEQYGRTDGLMTGARDLTWSHASLISASYAKAGSPAA

SEQ ID NO: 72 - protein, 583 aa, Circinella umbellata
APTSEIELDSYTYTGGVFSGRLYVKNIAYTKEVNVYWSDASEDWANNGNYVAATYSEAISGTNYEYWEFSAKIGSSGISESYIKYTVSGSTYYDNNGGKNYAITETTTPTITATPTSTTATTTISSTTTTATSIPTSVPSTVPEGNVTVTEWVNKQLKISWSDLLQNVNPSGTVTGFIAASLSTSNPDYFYCWTRDAAMVARVMTYMYNTTEAGDSSLESALKDYITFQINSMKTATACNCLGEPKFNTDGSGYSGPWGRPQNDGPAERATTMILFADSYLAQGGDTSYVTNTLKPAIYTNLDYVVGTWSNNCFDLWEEVNGVHIFTLAVMRKSLLDGADFAARNGDTSRVSGYQSTASSIKTKLESFWSSSNNYITVTQSYSGGVQKAGLDVSTLIAANQGSMGDGFYTPGSDKMLATAVAIENSFANIYTINKNKPSWLGTAIGRYPEDTYNGNGNGQGNPWFIATATYAELYYRAILEWQQQESVTVNSVNFDFFSKFDSSAKVGTVYTPGTDTFNTMVSNVAFAADEFLSTMEHYAATNGSMSEQFNRETGSLTGARDLTWSHAAFITAGKAKLGIPSF

SEQ ID NO: 73 - protein, 585 aa, Mucor cordense
ETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSTITTWLKSQEDISRGAMLRNINPPGAATGFIAASLSTSGPDYYYAWTRDSALTSHVIAHDYNTTLAGNTTILNILKDYVTFSVKSQSVSTVCNCLGEPKFNPDGSSYTGAWGRPQNDGPAERASTFILFGDSYLKQTGDATYVTGTLAPAIYKDLDYVVNTWSNGCFDLWEEVNGVHFYTLMSMRRGLLDGANFAKRNGDTTRATTYTNTAASIATKIDTFWVSSGNYVQVSQSVTGGVSKAGYDCANILAANMAANHDGFYTPGSSKILATAVAIESKFASLYSINSGLASWLGTAIGRYPEDTYNGNGNSQGNPWFICTNAFAELYYRAIKEWTQAGSVTVDSTSLNFFKKFDSSAAAGTKYTVGTSAFTNLVQNIANGADKFLSTSKFHAATNGSMSEQYNRDSGLMTGARDLTWSHASLISASRAKAGSPSL

SEQ ID NO: 74 - protein, 590 aa, Phascolomyces articulosus
DVPTSANIELDYYTYKDKVFSGRIYVKNIAYEKTVAVYWSDASGDWNNNGNNVAASYSESISGTDYEYWDFSTTIGSGGIKQSYLKYTVSGNTYYDNNGSNNYDITETTATTTTTTTTTTTTGSTATTSTTSGPTSTTSTTPGPTPTNVPDGNVTVTEWVNTQFAISWPTLLKNVNPSGTVKGFIAASLSTNNPDYFYSWTRDSALVAHTMTYLYNTSEAGDSTIESALKDYVTFSINAMNAATVCNCLGEPKFNTDGSGYTGAWGRPQNDGPASRATTMILFADSFLAQGGDVSYVINTLKPAIYKDLDYVVSTWSNTCYDLWEEVNGVHIYTLSVMRRALIDGANFAQRNGDTSRVSGYTSTATTIKTRLESFWSDSNNYITVTQSYSGGVQKAGLDVSTLIAANIGSVGDGFYTPGSDKVLATAVAIEKSFANLYTINQNKPSWLGNAIGRYPEDTYDGYGNSKGNPWFIATATYAELYYRAILEWQQQASVTVNSINLGFFSKFDSSASVGTVYTPGTDSFANMVSNVAFAADEFLSTIDYHAMNNGSMHEQYNRDTGISQGARDLTWSHAAFITAAKAKLGAPAF

SEQ ID NO: 75 - protein, 578 aa, Rhizopus microsporus
VPTSAAVQVESYKYDGTTFSGRIFVKNIAYSKVVTVIYSDGSDNWNNNNNKISAAYSEAISGSNYEYWTFSAKLSGIKQFYVKYEVSGSTYYDNNGTKNYQVQATSATSTTATATTTTSTSTTTTSTGPTSTASVSFPTGNSTISSWIKNQEEISRFAMLRNINPPGSATGFIAASLSTAGPDYYYSWTRDSALTANVIAYEYNTTFAGNTTLLKYLKDYVTFSVKSQSVSTVCNCLGEPKFNADGSSYTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKNTASTMESKISSFWSDSNNYVQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAIKEWISNGKVTVSNISLPFFKKFDSSATSGKTYTAGTSDFNNLAQNIALGADRFLSTVKFHAYNNGSLSEEYDRSTGMSTGARDLTWSHASLITAAYAKAGSPAA

SEQ ID NO: 76 - protein, 443 aa, Spinellus fusiger
VPSGNSTITAWGSKQDGISFSTMLGNINPPGSSKGFIAASLSTAGPNYYYSWTRDSALVARAITYKYSTSYQNDPKILGLLKDYVTYQVNEQTESTVCNCLGEPKFNPDGSSFSGPWGRPQNDGPAERASTMILFAKSYYAQTNDVGYVSNTLKPAIYKDLDYIVNVWGNNCFDLWEEVNGVHFYTLMMMRHGLVQGSIFANTLGDSTRANTYKTAAQNIKNRIDTFWESGSNYIVVTQNQSAGVNKPSGLDVAVLLAANQGGLGDGVYTPGSDKVLATAVALEKSFASLYPINKNLPSYYGTAIGRYPEDTYNGNGNSEANPWFIATTTYAELYYRAISEWTNGSGVTVNSINKEFFSKFDASATNGKVYTPGSDSFNSLVNNVAIAADNFLSTVRYHQTSNGSLSEQFNRYTGFMTGARDLTWSHAAMVTALAAKAGTPSP

SEQ ID NO: 77 - protein, 588 aa, Syncephalastrum racemosum
ATVPTTQVQLDYYTYSNNVLSGRIYVQNIAYSKVVKVIYSDASGNWNNNGNTISASYVESISGTNYEYWDFSATIGTAGIKQFYLRYDVSGSTYYDNNGGDNNNYNVVATTSTTSSSTTTTTATTTTATKTTSTTSATATPTSSATFPSGNSTITTWAKSQKDISWKTLLTSLNPSGTAKGFIAASLSTSNPDYYYAWTRDSALVARTMVNMYNTTEAGSASVLGLLQDYVTFQINAMSTSTVCNCLGEPKFNPDGSSYTGAWGRPQNDGPAERASTFILFADSYIAQGGQLSYVTGTLAPAIYKDLNYVVSTWSNNCFDLWEEVNGRHMFTLAVMRRALLDGVNFASRIGDTTYSSTWSSTASSIQSTLSGYYLSSGNYITTVQNFQSGVSKAGYDVSTLIAANVAGMGDGFFTPGSDEVLATAVAIENKFSSLYGVNANKASYLGTAIGRYPEDTYNGYGNGQGNPWFIATAAYAELYYRAILEWQTKSSIVVNSKNLGFFSKFDSSAAVGTTYTPGTTAYSNMVQNVALAADRFLSTVQLHAATNGSMSEQFNRDTGVMQGARDLTWSHSAFITAARAKLGSPVY

SEQ ID NO: 78 - protein, 448 aa, Syncephalastrum racemosum
ANSTVPDGNSTTTAWVKKQEAISWTDLKTNVNPEGAAKGFIAASLSTSEPDYYYAWTRDSALVARVMVNKYNTTDAGDANLLGLLQDYVSFQINAMGESTVCNCLGEPKFNPDGSSYTGAWGRPQNDGPAERASTFILLADSMIAQKSANGSYVSDTLAPAIYKDLAYVASTWENACYDLWEEVNGKHMYTLSVMRRALLDGADFASRQGQTANVTSWKSTADKIKSSLEGFFSSDNGYIEVTQDMQGGVQKKGLDVSTLIAANIGSMGDGFYTPGSDEVLATAVAVEAAFADLYKINQNTNSTGLGTAIGRYPEDTYNGVGNSQGNPWFIATNTFAELYYRAILEWQNKGTITVNSVNAAFFSKFDSSAKAGTTYKSGSTEFDSLINKVALAADAFLNTVQTYAASNGSMSEQYNRDTGALTGARDLTWSHASLITAANAKLGTPYN

SEQ ID NO: 79 - protein, 452 aa, Thermomucor sp.
VVTAAAAASVPSGNATITSWIQEQLAISWNTMLTSVNPVGAVTGFIAASLSTANPDYYYCWTRDAALVARVMTFMYNTTQAGDSGLLGILQDYVSFQIHAMGESTVCNCLGEPKFNPDGSSYTGAWGRPQNDGPAERASTFIKIADSYLTQTGDVSYVTNTLKPAIYEDLDYIVNVWQNTCFDLWEEVYGMHMYTLAVMRRGLLDGADFATRNGDTDKASTYTSTATSIQTRLATFWSDSNGYITVTQDYQSGVSKAGLDVSTLIAANVAGMNDGFFTPGSDEILATAVKVEAAFANLYGININKASYLGNAIGRYPEDTYNGNGNSEGNPWFIATAAFAELYYRAIMEWQLRGSSITVNSVNQGFFTKFDPSATAGTTYTPGTDAFNSLIDNVALAADQFFSTIHLHRATNGSMSEQYNRDTGFMQGARDLTWSHAAFITAAKAKQGTPSF

SEQ ID NO: 80 - protein, 589 aa, Zychaea mexicana
VDVPSVPIQLESYTYSENVFAGRIFVQNIAYTKEVNVFWSDASDDWNDNGNSVAASYSESIADTNYEYWEFSTTIGSAGISQSYLRYDVSGSIYYDNNDSQNYDITETSTPTTTTAAPTSTTSTPTTTTDGGSTTTTTATSVPTSTGVPEGNATISEWASAQLDISWPNLMMNVNPSGAVTGSIVASLSTSNPDYFYIWTRDAAMVARVMVYMYNTTEAGDTNLLNALTDYVTFSISSMNVDTVCDCLGEPKFNVDGSGYTGAWGRPQNDGPAERASTMILIADSYIAQGGDVSYVTDTLKPAIYTDLDYVVDTWSNVCFDLWEEVNGIHMYTLSVMRKALLDGANFATRNGDTSRVSGYESTASSIKTRLESFWSSSNNYITVTQSFSGGVSKAGLDVSTLIAANLASMNDGFYTPGSDEILATAVAIENSFISEYTLNQNRPSWLSTAIGRYPEDSYDGYGNSQGNPWFIATATYAELYYRAILEWQQQASISVNSVNLGFFSKFDSDASVGTVYTPGTEDFANMVSNVAFAADEFLATIENHSAVNGSLSEQYNRDTGIMQGARDLTWSHAAFITAAKAKQGAPIH

SEQ ID NO: 81 - protein, 432 aa, Saksenaea vasiformis
WVSKQEDISFSEMLRNVNPEGTAKGFVAASLSTAGPDYFYTWTRDAALVSRVIAYKYNTTNAGDSKIHGVLDDYVNFQINTQSESTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 82 - protein, 432 aa, Backusella circina
WSPSQHSISLYAMLRNINPPGSAAGFISASLSTSGPDYYYSWTRDSALVAHVIVNEYNTTYQGNSTLLGILKDYVTYSLNAQTTSTVCNCLGEPKFNPDGSSFTGAWGRPQNDGPAERAVSFIYFADSYLTQTSDSSYVTGTLAPAIYKDLDYVVSVWSNGCFDLWEEVNGIHFYTLMFMRRGLLDGANFASRNGDSTRASTYTSTAASIKTKIDGFWVSSGNYVQVSQSVTSGVSKAGYDASTLIAANQASRGDGFYTPGSDKMLATAVAIESQFSSLYSINTNKASYLGNAIGRYPEDTYNGNGNSQGNPWFLCTNAFGELFYRAISEWNAAGSVTVNSVNLAFFKKYDSSTSSGTTYTVGTSAYNNLVQNVALAADAYFSTVKYHALTNGSMSEQYDRSSGMATGARDLTWSHAAMITALKAKSGTPVY

SEQ ID NO: 83 - protein, 432 aa, Backusella circina
WIKGQEDTSRSVMLGNINPPGSATGFISASLSTSGPDYYYHWTRDAALVAHVIVNDYNTTLSGDSSTLQVIKDYVTFSVNSQSESTVCNCLGEPKFNPDGSSYTGAWGRPQNDGPAERATTFIFFADTYLAQGGDSSYVTGTLAPAIYADLDYVVNNWSTGCYDLWEEVNGIHFYTLMVMRRGLIDGASFASRNGDSTRSSSYTSTAKSIATKIDSFWSASNNYVAVSQSVTSGVSKAGYDASTIIAANQASLGDGFYTPGSDKMLATSVAVENAFSSLYTVNTGKASYLGNAIGRYPEDTYNGNGNSQGNPWYLCTNALGELYYRAIKEWNAAGSVTVNSVNLGFFQKIDSSISSGTTFTLGTSDYSTLVDNVALAADKFFAVVQYHERSNGSMPEQFGREDGLPTGARDLTWSHAAMISAARAKAGTPVY

SEQ ID NO: 84 - protein, 432 aa, Benjaminiella poitrasii
WLKSQIGISRYAMLRNINPAGSAVGFIAASLSTANPDYYYAWTRDSALTSYVIANDYNSTLAGNSTILQIMKDYVTFSVKSQSVSTACNCLGEPKFNPDGSSYTGAWGRPQNDGPAERATTFILFADSYLKQTGDASYVTGTLKPAIFKDLDYVVNTWTNGCFDLWEEVNGVHFYTLMVMRKGLIRGANFATRNGDSTRASTYTNTAASIKTKMDSFWSSGNNYIAVSQSVTGGVSKAGYDVANIIAANVGSLQDGVYTPGSDRILATAVAVEAKFASLYGVNSNLPGYLGNSIGRYPEDTYNGNGNSQGNPWFLATNAYAELYYRAIREWYDNGGVTVNSVNLPFFKKFDSSAASGTTYTVGTTAFNTMVSNVAAAADKFFSTVKFHAYTNGSMSEQFGRNDGLCTGARDLTWSHASLISAALAKAGTPSV

SEQ ID NO: 85 - protein, 432 aa, Choanephora cucurbitarum
WIDKQIDISRSAMLKNINPAGTVKGFIAASLSTSNPDYFYAWTRDAALVAHVVANDYNRTKSGDATYLGLLKDYVTFSINSQNTPTACNCLGEPKFNKDGSGYNGPWGRPQNDGPAERADTFVLIADSILTQTKDVSYVTGTLRPAIYTDLDYVVRTWSNGCFDLWEEVNGVHFYTLMVMRRALLVGANFASRNGDSARASNYNNAANSIKSKIDSFWSSNNNYVAVSQSVTGGVSKAGYDVSTLIAANVGSLSDGFYTPGSERMLATAVAIEDKFANLYGINRNLDSSLGNAIGRYPEDTYNGNGNSQGNPWFIATNAYAELYYRAIKEWNNNGGVTVTNVNLNFFKKFDGSASVGTKYTAGSAAYNTLTQNIALAADKFFNTVKVHAATNGSMSEQYHRDTGSMTGARDLTWSHASLITAALAKAGTPVA

SEQ ID NO: 86 - protein, 432 aa, Rhizopus stolonifer
WIKRQEKISRFAMLRNINPPGSAAGFIAASLSTSGPDYYYSWTRDSALTSNLIAYEYNTTLSGNTTILNILKDYVKFSISSQTTSTVCNCLGEPKFNHDGSSYTGAWGRPQNDGPAERANTFILFADSYLDQTKDASYVTGTLKPAIFKDLDYVVNVWSNGCYDLWEEVNGVHFYTLMVMRKGLLLGADFAKRNGDSTRASTYTNTASTIATKISSFWVSSSNWIQVSQSVTAGVSKKGLDVSTLLAANLGSVEDGFFTPGSEKILATAVAIEDAFASLYPINSNLPSYLGNSIGRYPEDTYNGNGNSQGNPWFLAVNGFAELYYRAIKEWVNNGSVTVSNISLSFFKKFDSSATAGKTYTAGTADFNNLAQNIALGADRFLSTVQTHAFNNGSLAEEYDRTTGVSTGARDLTWSHASLITAAYAKAGSPAA

SEQ ID NO: 87 - protein, 432 aa, Mucor circinelloides
WIDGQTSVSRYAMLRNINPAGAVSGFIAASMSTSGPDYFYAWTRDSALTSHVVAYDYNTTLAGNSTILGLLKNYVTFSINSQTTSTVCNCLGEPKFNKDGSSYTGAWGRPQNDGPASRADTFILIADSILKQTGDATYVTGTLAPAIYKDLDYVVSTWSNGCFDLWEEVNGVHFYTLMVMRRGLIKGANFASRNGDNTRANTYTNTAASIKTKIDSFWNSNGNYVTVSQSVTGGVSKAGYDASVLIAANLGSVQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSAFNSLVQNVAVAADAFFSTVKFHAATNGSMSEQYGRSDGLMTGARDLTWSHASLISASYAKAGSPAA

SEQ ID NO: 88 - protein, 431 aa, Dichotomocladium elegans
WADSQHKISWKAMLANINPPGSATGFIAASLSTSGPDYYYAWTRDAAMVAHVIVNAYNTTKAGDATTLGVLKDFVTFQIKAMATSTVCNCLGEPKFNPDGSSYTGPWGRPQNDGPAERATTFILFADSYLSQTGDAAYVTTLKRAIFTDLDYVVTTWQDNCFDLWEEVNGLHMYTLAVMRRSLVDGASFAARNGDNTRASTYTNTAKSIETKLASFYNSSSNYVVVTKNFAGGVNKAGLDTSTLIAANTAGLGDGFFTPGSPEILATAAAIEKSFADLYGINKQIPNWLGTAIGRYPEDTYNGNGNSQGNPWFICTATFAELYYRAILEWQHAGSVTVNNVNLPFFKKFDSSTSSGTTYTVGTSSFDSLISKVAYAADNFFSTIKYHAATNGSMSEQFNRDTGFMTGARDLTWSHAAFITAAKAKAGTPVY

SEQ ID NO: 89 - protein, 432 aa, Fennellomyces sp.
WVNDQLEISWPSLLKNVNPSGAVTGFIAASLSTNDPDYFYCWTRDAALVARVMVYMYNTTEAGDTSLRSKLQDYVTFQINSMKTATVCNCLGEPKFNKDGSGYSGAWGRPQNDGPADRAITLILFADSFIAQGGDVSYITNTLKPAIYTNLDYVVNTWSNVCFDLWEEVNGVHIYTLSVMRKGLLEGADFASRNGDSTRANTYRSTASSIKTRLESFWSSSNNYITVTQSYSGGVNKAGLDVSTLLAANGASMNDGFYTPGSDKMLATAVAIENSFAGIYAVNQNRPDWKGTAIGRYPEDTYDGHGNSQGNPWFIATAAYAEMYYRAILEWQQQPSITVNSINLSFFKKFDSSAAVGTVYKPGTQAFNNMVSNVAFAADEFFSTMNFHSATNGSMSEQYNRNTGIMQGARDLTWSHAAFITAAKAKLGTPVF

SEQ ID NO: 90 - protein, 432 aa, Gilbertella persicaria
WLKGQIETSRFAMLRNINPAGTVKGFIAASLSTANPDYFYAWTRDAALVGHVIANDYNRTLAGNSTYLGLLKDYVTFSVNSQSTSTICNCLGEPKFNKDGSGYSGAWGRPQNDGPAERADTFILIADSILKQTGDATYVTGTLRPAIYKDLDYVVNVWSNGCFDLWEEVNGVHFYTLMVMRRSLILGANFASRNGDSTRASTYTNTANSIKTKIDTFWSSSNNYVAVSQSVTGGVNKAGYDVANLIAANVGSLDDGFYTPGSERILATAVAVESKFASIYGVNQNLPSWLGNSIGRYPEDTYNGNGNSQGNPWFIATNTYAELYYRAIKEWTNNGGVTVTNVNFNFFKKFDSSASVGTKYTVGTSAFNTLTQNVALAADNFFSTVKVHAATNGSMSEQFGRDSGVMTGARDLTWSHASLITAALAKTGAPVA

SEQ ID NO: 91 - protein, 432 aa, Mucor circinelloides
WIDGQTSVSRYAMLRNINPAGAVTGFIAASMSTSGPDYFYAWTRDSALTSHVVAYDYNTTLAGNSTILGLLKNYVTFSLNSQTTSTVCNCLGEPKFNKDGSGYSGAWGRPQNDGPASRADTFILIADSILKQTGDATYVTGTLAPAIYKDLDYVVSTWSNGCFDLWEEVNGVHFYTLMVMRRGLVKGANFASRNGDSTRATTYTNTAASIKTKIDSFWNSNGQYVSVSQSVTGGVSKAGYDASVLIASNLGSLQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSSFNSLVQNVAVAADAFFSTIKFHAATNGSMSEQYGRTDGLMTGARDLTWSHASLISASYAKAGSPAA

SEQ ID NO: 92 - protein, 432 aa, Circinella umbellata
WVNKQLKISWSDLLQNVNPSGTVTGFIAASLSTSNPDYFYCWTRDAAMVARVMTYMYNTTEAGDSSLESALKDYITFQINSMKTATACNCLGEPKFNTDGSGYSGPWGRPQNDGPAERATTMILFADSYLAQGGDTSYVTNTLKPAIYTNLDYVVGTWSNNCFDLWEEVNGVHIFTLAVMRKSLLDGADFAARNGDTSRVSGYQSTASSIKTKLESFWSSSNNYITVTQSYSGGVQKAGLDVSTLIAANQGSMGDGFYTPGSDKMLATAVAIENSFANIYTINKNKPSWLGTAIGRYPEDTYNGNGNGQGNPWFIATATYAELYYRAILEWQQQESVTVNSVNFDFFSKFDSSAKVGTVYTPGTDTFNTMVSNVAFAADEFLSTMEHYAATNGSMSEQFNRETGSLTGARDLTWSHAAFITAGKAKLGIPSF

SEQ ID NO: 93 - protein, 432 aa, Mucor cordense
WLKSQEDISRGAMLRNINPPGAATGFIAASLSTSGPDYYYAWTRDSALTSHVIAHDYNTTLAGNTTILNILKDYVTFSVKSQSVSTVCNCLGEPKFNPDGSSYTGAWGRPQNDGPAERASTFILFGDSYLKQTGDATYVTGTLAPAIYKDLDYVVNTWSNGCFDLWEEVNGVHFYTLMSMRRGLLDGANFAKRNGDTTRATTYTNTAASIATKIDTFWVSSGNYVQVSQSVTGGVSKAGYDCANILAANMAANHDGFYTPGSSKILATAVAIESKFASLYSINSGLASWLGTAIGRYPEDTYNGNGNSQGNPWFICTNAFAELYYRAIKEWTQAGSVTVDSTSLNFFKKFDSSAAAGTKYTVGTSAFTNLVQNIANGADKFLSTSKFHAATNGSMSEQYNRDSGLMTGARDLTWSHASLISASRAKAGSPSL

SEQ ID NO: 94 - protein, 432 aa, Phascolomyces articulosus
WVNTQFAISWPTLLKNVNPSGTVKGFIAASLSTNNPDYFYSWTRDSALVAHTMTYLYNTSEAGDSTIESALKDYVTFSINAMNAATVCNCLGEPKFNTDGSGYTGAWGRPQNDGPASRATTMILFADSFLAQGGDVSYVINTLKPAIYKDLDYVVSTWSNTCYDLWEEVNGVHIYTLSVMRRALIDGANFAQRNGDTSRVSGYTSTATTIKTRLESFWSDSNNYITVTQSYSGGVQKAGLDVSTLIAANIGSVGDGFYTPGSDKVLATAVAIEKSFANLYTINQNKPSWLGNAIGRYPEDTYDGYGNSKGNPWFIATATYAELYYRAILEWQQQASVTVNSINLGFFSKFDSSASVGTVYTPGTDSFANMVSNVAFAADEFLSTIDYHAMNNGSMHEQYNRDTGISQGARDLTWSHAAFITAAKAKLGAPAF

SEQ ID NO: 95 - protein, 432 aa, Rhizopus microsporus
WIKNQEEISRFAMLRNINPPGSATGFIAASLSTAGPDYYYSWTRDSALTANVIAYEYNTTFAGNTTLLKYLKDYVTFSVKSQSVSTVCNCLGEPKFNADGSSYTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKNTASTMESKISSFWSDSNNYVQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAIKEWISNGKVTVSNISLPFFKKFDSSATSGKTYTAGTSDFNNLAQNIALGADRFLSTVKFHAYNNGSLSEEYDRSTGMSTGARDLTWSHASLITAAYAKAGSPAA

SEQ ID NO: 96 - protein, 433 aa, Spinellus fusiger
WGSKQDGISFSTMLGNINPPGSSKGFIAASLSTAGPNYYYSWTRDSALVARAITYKYSTSYQNDPKILGLLKDYVTYQVNEQTESTVCNCLGEPKFNPDGSSFSGPWGRPQNDGPAERASTMILFAKSYYAQTNDVGYVSNTLKPAIYKDLDYIVNVWGNNCFDLWEEVNGVHFYTLMMMRHGLVQGSIFANTLGDSTRANTYKTAAQNIKNRIDTFWESGSNYIVVTQNQSAGVNKPSGLDVAVLLAANQGGLGDGVYTPGSDKVLATAVALEKSFASLYPINKNLPSYYGTAIGRYPEDTYNGNGNSEANPWFIATTTYAELYYRAISEWTNGSGVTVNSINKEFFSKFDASATNGKVYTPGSDSFNSLVNNVAIAADNFLSTVRYHQTSNGSLSEQFNRYTGFMTGARDLTWSHAAMVTALAAKAGTPSP

SEQ ID NO: 97 - protein, 432 aa, Syncephalastrum racemosum
WAKSQKDISWKTLLTSLNPSGTAKGFIAASLSTSNPDYYYAWTRDSALVARTMVNMYNTTEAGSASVLGLLQDYVTFQINAMSTSTVCNCLGEPKFNPDGSSYTGAWGRPQNDGPAERASTFILFADSYIAQGGQLSYVTGTLAPAIYKDLNYVVSTWSNNCFDLWEEVNGRHMFTLAVMRRALLDGVNFASRIGDTTYSSTWSSTASSIQSTLSGYYLSSGNYITTVQNFQSGVSKAGYDVSTLIAANVAGMGDGFFTPGSDEVLATAVAIENKFSSLYGVNANKASYLGTAIGRYPEDTYNGYGNGQGNPWFIATAAYAELYYRAILEWQTKSSIVVNSKNLGFFSKFDSSAAVGTTYTPGTTAYSNMVQNVALAADRFLSTVQLHAATNGSMSEQFNRDTGVMQGARDLTWSHSAFITAARAKLGSPVY

SEQ ID NO: 98 - protein, 434 aa, Syncephalastrum racemosum
WVKKQEAISWTDLKTNVNPEGAAKGFIAASLSTSEPDYYYAWTRDSALVARVMVNKYNTTDAGDANLLGLLQDYVSFQINAMGESTVCNCLGEPKFNPDGSSYTGAWGRPQNDGPAERASTFILLADSMIAQKSANGSYVSDTLAPAIYKDLAYVASTWENACYDLWEEVNGKHMYTLSVMRRALLDGADFASRQGQTANVTSWKSTADKIKSSLEGFFSSDNGYIEVTQDMQGGVQKKGLDVSTLIAANIGSMGDGFYTPGSDEVLATAVAVEAAFADLYKINQNTNSTGLGTAIGRYPEDTYNGVGNSQGNPWFIATNTFAELYYRAILEWQNKGTITVNSVNAAFFSKFDSSAKAGTTYKSGSTEFDSLINKVALAADAFLNTVQTYAASNGSMSEQYNRDTGALTGARDLTWSHASLITAANAKLGTPYN

SEQ ID NO: 99 - protein, 433 aa, Thermomucor sp.
WIQEQLAISWNTMLTSVNPVGAVTGFIAASLSTANPDYYYCWTRDAALVARVMTFMYNTTQAGDSGLLGILQDYVSFQIHAMGESTVCNCLGEPKFNPDGSSYTGAWGRPQNDGPAERASTFIKIADSYLTQTGDVSYVTNTLKPAIYEDLDYIVNVWQNTCFDLWEEVYGMHMYTLAVMRRGLLDGADFATRNGDTDKASTYTSTATSIQTRLATFWSDSNGYITVTQDYQSGVSKAGLDVSTLIAANVAGMNDGFFTPGSDEILATAVKVEAAFANLYGININKASYLGNAIGRYPEDTYNGNGNSEGNPWFIATAAFAELYYRAIMEWQLRGSSITVNSVNQGFFTKFDPSATAGTTYTPGTDAFNSLIDNVALAADQFFSTIHLHRATNGSMSEQYNRDTGFMQGARDLTWSHAAFITAAKAKQGTPSF

SEQ ID NO: 100 - protein, 432 aa, Zychaea mexicana
WASAQLDISWPNLMMNVNPSGAVTGSIVASLSTSNPDYFYIWTRDAAMVARVMVYMYNTTEAGDTNLLNALTDYVTFSISSMNVDTVCDCLGEPKFNVDGSGYTGAWGRPQNDGPAERASTMILIADSYIAQGGDVSYVTDTLKPAIYTDLDYVVDTWSNVCFDLWEEVNGIHMYTLSVMRKALLDGANFATRNGDTSRVSGYESTASSIKTRLESFWSSSNNYITVTQSFSGGVSKAGLDVSTLIAANLASMNDGFYTPGSDEILATAVAIENSFISEYTLNQNRPSWLSTAIGRYPEDSYDGYGNSQGNPWFIATATYAELYYRAILEWQQQASISVNSVNLGFFSKFDSDASVGTVYTPGTEDFANMVSNVAFAADEFLATIENHSAVNGSLSEQYNRDTGIMQGARDLTWSHAAFITAAKAKQGAPIH

SEQ ID NO: 101 - protein, 609 aa, Mucor ambiguus
MKFSSLLKKPLLLIAGILAATVVAETVPTTAEVKVKSFTYDGSTFAGQIYIKNIAYTKTVTVIYSDGSNNWNNNGNTIAASYSAAISGSNYEYWTFSAPVSSIKQFYVKYVVSGTTYYDNNSSGNYQVSTTTTTTTAPTTTTSGGSSTTTGGSTTTATSVPTAVPSGFPTGNSTISSWIDGQTSVSRYAMLRNINPAGTVTGFIAASMSTSGPDYFYAWTRDSALTSHVVAYDYNTTLAGNSTILGLLKNYVTFSLNSQTTSTVCNCLGEPKFNKDGSSYTGAWGRPQNDGPASRADTFILIADSILKQTGDATYVTGTLAPAIYKDLDYVVSTWSNGCFDLWEEVNGVHFYTLMVMRRGLVKGASFASRNGDSTRANTYTNTAASIKTKIDSFWNSNGQYVSVSQSVTGGVSKAGYDASVLIASNLGSLQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSAFNSLVQNVAVAADAFFSTIKFHAATNGSMSEQYGRTDGLMTGARDLTWSHASLISASYAKAGSPAA

SEQ ID NO: 102 - protein, 605 aa, Rhizopus microsporus
MKLMNPSIKACVFFILSYLSLLVSSAAVPTSAAVQVESYKYDGTTFSGRIFVKNIAYSKVVTVIYSDGSDNWNNNNNKISAAYSEAISGSNYEYWTFSAKVSGIKQFYVKYEVSGSTYYDNNGTKNYQVQATSATSTTATATTTTTTSTPATSTGPANNAPVSFPTGNSTISSWIKKQEEISRFAMLRNINPPGSATGFIAASLSTAGPDYYYSWTRDSALTANVIAYEYNTTFTGNTTLLKYLKDYVTFSVKSQSVSTVCNCLGEPKFNADGSSYTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKKTASTMESKISSFWSDSNNYIQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAISEWISNGKVTVSNISLPFFKKFDPSATSGKTYTAGTSDFDNLAQNIALGADRFLSTVKFHAYTNGSLSEEYDRSTGMSTGARDLTWSHASLITAAYAKAGSPAA

SEQ ID NO: 103 - protein, 605 aa, Rhizopus azygosporus
MKLMNPSMKACVFFILSYFSLLVSSAAVPTSAAVQVESYNYDGTTFSGRIFVKNIAYSKVVTVIHSDGSDNWNNNNNKVSAAYSEAISGSNYEYWTFSAKLSGIKQFYVKYEVSGSTYYDNNGTKNYQVQATSATSTTATATTTTATGTTTTSTGPTSTASVSFPTGNSTISSWIKNQEEISRFAMLRNINPPGSATGFIAASLSTAGPDYYYSWTRDSALTANVIAYEYNTTFTGNTTLLKYLKDYVTFSVKSQSVSTVCNCLGEPKFNADGSSFTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKKTASTMESKISSFWSDSNNYIQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAIKEWISNGKVTVSNISLPFFKKFDSSATSGKTYTAGTSDFNNLAQNIALGADRFLSTVKFHAYTNGSLSEEYDRSTGMSTGARDLTWSHASLITAAYAKAGSPAA

SEQ ID NO: 104 - protein, 468 aa, Saksenaea vasiformis
MPSWKTLFLLLGPIATAAAAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 105 - protein, 640 aa, Aspergillus niger
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 106 - protein, 631 aa, Aspergillus fumigatus
MPRLSYALCALSLGHAAIAAPQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTAAETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWQ

SEQ ID NO: 107 - protein, 567 aa, Fibroporia radiculosa
MLFLLAALGLACSAAAQSTSVSAYIASESPVAKAGVLANIGTEGSLSSGAYSGVVIASPSTVNPDYLYTWVRDSSLTFQALIDQYVYGEDPTLRSLIDEFITAESILQQTTNPSGTVSTGGLGEPKFNINETAFTGPWGRPQRDGPALRSTAIITYATYLWNSGNTSYVSDSLWPIIELDLNYIATYWNFSTFDLWEEIDSSSFWTTAVQHRALRQGITFANLIGQTSPVSNYETQAGDILCFLQTYWNPTGNYMTANTGGGRSGKDSNTVLASVHTFDPDAGCDSTTFQPCSDKALSNLKVYVDSFRSLYAINDGIASDAAVATGRYPEDVYYGGNPWYLCTFAVAEQLYDALIVWSSQGYLEITDLSLAFFQQFDSDVGTGTYDSGSSTYSTLTSAIRTFADGFVLTNAKYTPTNGSLSEEYTSADGTPISAYDLTWSYASALTVFAAEAGTTYGSWGAAGLTVPSTCTSGVAVTFEVDYDTEYGENVYITGSVNALENWSATNALIMSAADYPTWSITVYLPPSTTIQYKYLTQYNGEVTWEDDPNNEITTPASGSMTQVDSWH

SEQ ID NO: 108 - protein, 582 aa, Fusarium verticillioides
MFTQILYGLTALSALQGQVTASPGGSSLDRFISKEADISIKGVLANIGADGKRAQGAAPGAVVASPSRTDPDYWYTWTRDSALTYKVLVERFIHGDKSLQRKIDEYVSAQAKLQGVTNPSGGPESGGLGEPKFHVNLTAFTGSWGRPQRDGPPLRATALTLYANWLVSHGDRSKAVNKVWPVIEKDLAYTVKFWNRTGYDLWEEVNGSSFFTLSASHRALVEGAALAKKLGKSCSDCATNAPRVLCFMQSFWTGSYIDSNINVNDGRKGLDANSILSSIHTFDPSSKCTDSTFQPCSSRALANHKEVVDSFRSIYGVNKNRGKGKAAAVGRYSEDVYYDGNPWYLATLAAAEQLYAAVYQWNKIGSITVDSVSLPFFSDLVPKVSKGTYRKNSKTYKAIIKAVTSYADGFVAVVQTYTPKDGSLAEQFDKSTGTPKSAVHLTWSYASFVGAAERRTGVVPPAWGESNANKVPAVCEAAPACDTTITFNVKNVDVTSDQKVYIVGGITQLSNWAPADGIALEESTSTKGLWTVKVKIPSDTSFEYKYIKKTSDGTVTWESDPNNSAATGSKCGSSSTINDEWR

SEQ ID NO: 109 - protein, 576 aa, Gloeophyllum trabeum
MYRFLVCALGLLGTVLAQSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 110 - protein, 616 aa, Penicillium oxalicum
MRLTLLSGVAGVLCAGQLTAARPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 111 - protein, 632 aa, Trichoderma reesei
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 112 - protein, 569 aa, Wolfiporia cocos
MRLSLASVFALAGGALAQTTSVTSYIASESPIAKAGVLANIGADGSLSSGAYSGIVIASPSTVNPNYLYTWTRDSSLTFMELINQYIYGEDDTLRTLIDEFVSAEATLQQVTNPSGTVSTGGLGEPKFNINETAFTGPWGRPQRDGPALRATAIMAYATYLYENGNTSYVTDTLWPIIELDLGYVAEYWNESTFDLWEEIDSSSFFTTAVQHRALRAGVTFANLIGETSDVSNYQENADDLLCFLQSYWNPTGSYVTANTGGGRSGKDANTLLASIHTFDPDAGCNATTFQPCSDKALSNHKVYVDSFRSLYAINDDISSDAAVATGRYPEDVYYNGNPWYLCTLAAAEQLYDSLIVWKAQGYIEVTSLSLAFFQQFDASVSAGTYDSSSDTYTTLLDAVQTYADGFVLMVAQYTPANGSLSEQYAKADGSPTSAYDLTWSFAAALTAFAARDGKTYGSWGAADLSSTCSGSTDTVAVTFEVQYDTQYGENLYITGSVSQLEDWSADDALIMSSADYPTWSITVDLPPSTLIQYKYLTKYNGDVTWEDDPNNEITTPASGSYTQVDSWH

SEQ ID NO: 113 - skipped

SEQ ID NO: 114 - protein, 8 aa, Synthetic construct
  VAR_SEQ 5: note = Any amino acid
YNTTXAGD

SEQ ID NO: 115 - skipped

SEQ ID NO: 116 - protein, 9 aa, Synthetic construct
  VAR_SEQ 8: note = Any amino acid
STLIAANXA

SEQ ID NO: 117 - skipped

SEQ ID NO: 118 - protein, 7 aa, Synthetic construct
NGNGNSQ

SEQ ID NO: 119 - protein, 468 aa, Saksenaea oblongispora
MPSWKTLIFLLGPITAALAAPVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTESTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 120 - protein, 463 aa, Apophysomyces ossiformis
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLEDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 121 - protein, 462 aa, Apophysomyces elegans
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 122 - protein, 463 aa, Apophysomyces variabilis
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 123 - protein, 463 aa, Apophysomyces trapeziformis
MMMSWKSLLLLLGPIAAATAAIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPSF

SEQ ID NO: 124 - DNA, 1404 bp, synthetic construct
  misc_feature 1..1404: note = codon modified
  source 1..1404: mol_type = other DNA
atgccttcttggaagaccctgattttcctcctgggccctattaccgccgctctggctgctcctgttaacaagcagtctttccctaccggcaaccccgacatcagctcttgggtttctaagcaggagggcattagcttcagcgagatgctccgaaacgtcaaccctcccggcaccgctaagggcttcgttgctgctagcctcagcaccgccggccccgactacttctacacctggacccgagacgccgctctcgtctcccgcgtcatcgcttacaagtacaacaccaccaacgctggcgacaacaacattcacggcgctctgcaggactacgtcaccttccagattaacacccagaccgagtccaccccttgcaactgcctgggcgagcctaagttcaaccccgacggcagctctttcaccggcccttggggccgacctcagaacgacggccctgctgagcgcgctagcagcatgattctcattgctgacagcttcctcgcccagaccaaggacaccgcttacgttaccaacaccctgaagcctgccatttacaaggacctggactacgttgttaacacctggtctaacccttgcttcgacctgtgggaggaggttaacggcgtccacttctacaccctcatggttatgcgacgaggcctcctcgacggcgctgacttcgctacccgaaacggcgacgcctctaaggctagctcttactctgacgctgctagcaagatcaagacccgactggactctttctgggtcagcgacaagaactacattaccgttacccaggactacaaggagggcgtcaagaaggacaccggcctcgacgttagcaccctcattgctgccaacgttgccggcaaggacgacggcttctacacccccggcagcgacaagattctggctaccgctgttgccttcgagaacgccatggctaagctgtaccctctgaaccagaacctcgacagccacctgggcaactctattggccgataccctgaggacacctacaacggcaacggcaactcccagggcaacccttggttcctcgccaccaccgctttcaccgagctgtactaccgagccatcttggaatggaaggacagcggcgttaccgtcacccccgtttccaaggacttcttcgccaagttcgactccaacgccgttgccggcaagaagtactctcctaactctgaggagttcgccaccctgaccgctaacatcgctgctgccgctgaccgattcatgtctaccgttaagtaccaccagaaccctaacggctctctgtccgagcagttcgaccgccacagcggctacatgaccggcgcccgagacctgacctggtctcacgctgctttcattaccgccgcccaggctcgcgctggcacccctagcttc

SEQ ID NO: 125 - protein, 19 aa, synthetic construct
  REGION 1..19: note = signal peptide
MPSWKTLIFLLGPITAALA

SEQ ID NO: 126 - protein, 449 aa, Saksenaea oblongispora
APVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTESTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 127 - DNA, 1389 bp, synthetic construct
  misc_feature 1..1389: note = codon modified
  source 1..1389: mol_type = other DNA
atgatgatgtcttggaagtctctgctcctcctgctgggccctatcgccgctgctaccgctgccatccctaccggcaacagcagcctgaacgcttgggttagctcccaggaggacatctctttctccgtcatgctgggcaacattaaccctcccggcaccgtcaagggcttcgttgccgctagcctgtccaccgctggccccgactacttctactcttgggtccgagacagcgccctcgtttcccgcgtcgtcacccacaagtacaacaccaccgagaccggcaactctaccgttgccggcttcttggaggactacgttcacttccagattaacacccagaccgagtctaccgtttgcaactgcctgggcgagcctaagttcaaccccgacggcagctcttacaccggcccttggggccgccctcagaacgacggccccgctgagcgagctagcaccatgattctgattgccgactctttcctgacccagaccaagaacacctcttacgttgacaacaccctgaagcccgccatttacaaggacctggactacgttgttaacacctggtctaacccttgcttcgacctgtgggaggaggttaacggcattcacttctacaccctcatggttatgcgacgaggcctcctcgacggcgccaacttcgccacccgaaacggcgacacctctaaggctagctcttacagcagcaccgctagccagattcagaacaagattgactccttctggagctccaacaagaactacattattgttacccaggactaccagaacggcgtccagaagcctagcggcctcgacatcagcaccctcattgctgccaacgtcgctggcatgaacgacggcttctacacccccggctctgacaagatgctcgccaccgccgttgctattgagaacgccatggctaacctgtaccccctcaacaagaacctgccttcttacctgggcaacgctattggccgataccctgaggacacctacaacggcgacggcaactcccagggcaacccttggttcctcgccaccaccgctttcagcgagctgtactaccgagctctgctggagtggcaggagaccggcgttaccgttaccagcatttctaaggacttcttcagcaagttcgaccctaacgctagcgctggcaagaagtactctcccggctctaacgagttcgcttctctggctcagaacattgctagcgctgctgaccgattcctgtccaccgtcaactaccaccgaaactccaacggcagcctgtctgaggagttcgaccgaaacaccggctacatgaccggcgcccgagacctgacctggtctcacgccgctttcattaccgctggcctcgctcgcgctggcacccctagcttc

SEQ ID NO: 128 - protein, 20 aa, synthetic construct
  REGION 1..20: note = signal peptide
MMMSWKSLLLLLGPIAAATA

SEQ ID NO: 129 - protein, 443 aa, Apophysomyces ossiformis
AIPTGNSSLNAWVSSQEDISFSVMLGNINPPGTVKGFVAASLSTAGPDYFYSWVRDSALVSRVVTHKYNTTETGNSTVAGFLEDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 130 - DNA, 1386 bp, synthetic construct
  misc_feature 1..1386: note = codon optimized
  source 1..1386: mol_type = other DNA
atgatgatgtcttggaagtctctgctcctgctcctgggccctattgctgccgccaccgctgccatccctaccggcaactctagcctcaacgcttgggttagcagccaggaggacatttctttcagcgtcatgctgggcaacattaaccctcccggcaccgtcaagggcttcgttgctgctagcctgtccaccgctggccccgactacttctactcttgggttcgagacagcgctctcgtttctcgcgtcgttacccacaagtacaacaccaccgagaccggcaacagcaccgttgctggcttcctgcaggactacgttcacttccagattaacacccagaccgagtccaccgtttgcaactgcctgggcgagcctaagttcaaccccgacggcagctcttacaccggcccttggggccgccctcagaacgacggccccgctgagcgagctagcaccatgattctgattgctgactccttcctgacccagaccaagaacacctcttacgttgacaacaccctgaagcccgctatttacaaggacctggactacgtcgtcaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttacagcagcaccgcttctcagatccagaacaagattgactctttctggtctagctctaagaactacatcatggttacccaggactaccagaacggcattcagaagcctagcggcctcgacattagcaccctcatcgccgctaacgtcgctggcatgaacgacggcttctacacccccggctccgacaagatgctcgctaccgccgttgctattgagaacgccatggctaacctgtaccctctgaacaagaacctgccttcttacctgggcaacgccattggccgataccccgaggacacctacaacggcgacggcaactctcagggcaacccttggttcctcgccaccaccgctttcagcgagctgtactaccgcgctctcctggagtggcaggagaccggcgttaccgtcaccagcatttccaaggacttcttctctaagttcgaccctaacgctagcgctggcaagaagtactcccccggctctgacgagttcaactccctcgcccagaacattgccagcgctgccgaccgattcctgtctaccgttaactaccaccgaaactccaacggctctctgtctgaggagtacgaccgaaacagcggctacatgaccggcgcccgagacctgacctggtcccacgccgctttcattaccgccggcctcgcgagagctggcacccctagc

SEQ ID NO: 131 - protein, 20 aa, synthetic construct
  REGION 1..20: note = signal peptide
MMMSWKSLLLLLGPIAAATA

SEQ ID NO: 132 - protein, 442 aa, Apophysomyces elegans
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 133 - DNA, 1389 bp, synthetic construct
  misc_feature 1..1389: note = codon modified
  source 1..1389: mol_type = other DNA
atgatgatgtcttggaagtctctgctcctcctgctgggccctattgccgctgccaccgctgccattcctaccggcaacagcagcctcaacgcttgggttagctcccaggaggacatttctttcagcgtcatgctgggcaacattaaccctcccggcaccgttaagggcttcgttgccgctagcctgtctaccgccggccccgactacttctactcttgggtccgagacagcgccctcgtttcccgcgtcgtcacccacaagtacaacaccaccgagaccggcaacagcaccgttgctggcttcctgcaggactacgtccacttccagattaacacccagaccgagtccaccgtttgcaactgcctgggcgagcctaagttcaaccccgacggcagctcttacaccggcccttggggccgacctcagaacgacggccccgctgagcgcgctagcaccatgattctgattgctgactccttcctcgctcagaccaagaacacctcttacgttgacaacaccctgaagcctgccatttacaaggacctggactacgtcgttaacacctggtctaacccttgcttcgacctgtgggaggaggttaacggcattcacttctacaccctcatggttatgcgacgaggcctcctggacggcgccaacttcgccacccgaaacggcgacacctccaaggctagctcttacagcagcaccgctagccagatccagaacaagattgactctttctggtctagctccaagaactacatcatggttacccaggactaccagaacggcgttcagaagcctagcggcctcgacattagcaccctcatcgccgctaacgttgctggcatgaacgacggcttctacacccctggctctgacaagatgctcgccaccgccgttgctattgagaacgctatggctaacctgtaccctctgaacaagaacctgccttcttacctgggcaacgccattggccgataccccgaggacacctacaacggcgacggcaactcccagggcaacccttggttcctcgctaccaccgctttcagcgagctgtactaccgagctctgttagagtggcaggagaccggcgtcaccgttaccagcatctctaaggacttcttctccaagttcgaccctaacgctagcgccggcaagaagtactcccccggcagcgacgagttcaactctctcgcccagaacatcgccagcgccgctgaccgattcctgtctaccgtcaactaccaccgaaactctaacggctctctgtctgaggagtacgaccgaaactccggctacatgaccggcgcgagagacctgacctggtctcacgctgctttcattaccgctggcctcgcccgagctggcaccccttctttc

SEQ ID NO: 134 - protein, 20 aa, synthetic construct
  REGION 1..20: note = signal peptide
MMMSWKSLLLLLGPIAAATA

SEQ ID NO: 135 - protein, 443 aa, Apophysomyces variabilis
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 136 - DNA, 1389 bp, synthetic construct
  misc_feature 1..1389: note = codon modified
  source 1..1389: mol_type = other DNA
atgatgatgtcttggaagtctctgctcctcctgctgggccctatcgccgctgccaccgctgccatccctaccggcaacagcagcctcaacgcttgggttagctctcaggaggacatctctttcagcgtcatgctgggcaacatcaaccctcccggcaccgtcaagggcttcgttgccgctagcctgtctaccgctggccccgactacttctactcttgggtccgagacagcgctctcgtttctcgcgttgttacccacaagtacaacaccaccgagaccggcaacagcaccgttgctggcttcctgcaggactacgttcacttccagattaacacccagaccgagtccaccgtttgcaactgcctgggcgagcctaagttcaaccccgacggcagctcttacaccggcccttggggccgccctcagaacgacggccccgctgagcgcgctagcaccatgattctgatcgctgactccttcctgacccagaccaagaacacctcttacgttgacaacaccctgaagcccgctatttacaaggacctcgactacgtcgttaacacctggtctaacccttgcttcgacctgtgggaggaggttaacggcattcacttctacaccctcatggttatgcgacgaggcctcctggacggcgccaacttcgccacccgaaacggcgacaccagcaaggctagctcttactccagcaccgcttcccagattcagaacaagattgactctttctggagcagctccaagaactacattattgttacccaggactaccagaacggcgtccagaagcctagcggcctcgacgtcagcaccctcattgctgccaacgtcgctggcatgaacgacggcttctacacccccggcagcgacaagatgctcgccaccgccgttgccattgagaacgccatggctaacctgtaccctctgaacaagaacctgccttcttacctgggcaacgctattggccgataccccgaggacacctacaacggcgacggcaactcccagggcaacccttggttcctcgccaccaccgctttcagcgagctgtactaccgagctctgcttgaatggcaggagaccggcgtcaccgttaccagcatttctaaggacttcttctctaagttcgaccctaacgctagcgccggcaagaagtactcccccggctccaacgagtacgctagcctcgcccagaacattgctagcgccgctgaccgattcctgtctaccgttaactaccaccgaaaccctaacggctctctgtctgaggagtacgaccgaaacaccggctacatgaccggcgcccgagacctgacctggtctcacgctgctttcattaccgccggcctggccagggctggcaccccttctttc

SEQ ID NO: 137 - protein, 20 aa, synthetic construct
  REGION 1..20: note = signal peptide
MMMSWKSLLLLLGPIAAATA

SEQ ID NO: 138 - protein, 443 aa, Apophysomyces trapeziformis
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPSF

SEQ ID NO: 139 - DNA, 1404 bp, synthetic construct
  misc_feature 1..1404: note = Artificial Sequence
  source 1..1404: mol_type = other DNA
atgccttcttggaaaaccctgttcctcctcctgggccctatcgccaccgctgccgctgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgcctcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttc

SEQ ID NO: 140 - protein, 449 aa, synthetic construct
  REGION 1..449: note = Artificial Sequence
APVDKQSLPTGNSTISSWVSKQEDISFSEMLRNVNPEGTAKGFVAASLSTAGPDYFYTWTRDAALASRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 141 - protein, 449 aa, synthetic construct
  REGION 1..449: note = Artificial Sequence
APVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 142 - protein, 468 aa, Saksenaea vasiformis
MPSWKTLFLLLGPIATAAAAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSESTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF